SEQUENCE LISTING (122 sequences)

SEQ ID NO: 1 - DNA, 2346 bp, Aspergillus oryzae
  misc_feature (31)..(31): n is a, c, g, or t
ttacttcaccaggatttagggtcgagttccntcggtgccgaaaagaatgcccgagcaatgtatttatgtggccccaggacagtttaattgccgatatccaagcctttcaggtgagtaaattgcagagcgtgtgacaagggtaaccaggagaatactccgcattttgtggggaaccccatgggacgatctttgggatgtggagacactcatttgaaaatgacagtgacttgtccagtcagcgctgctgaaaattgtctccctaatcccggcttttccttgtcgaaaatgattggggagtgcgtcacgtcacggccaagctttcctgcttaggaatttcctaagctaatacatggtaccttcctccggtcaaacttcggagaagccctagataagggcacgggatatagtccgatcttcatgtaccgacggattgaaagtttgaaacgctaaatgacatgttccttagtactgtcagcagtctccggtatctccgaggcagctacatatataaagtcaccaagctcacggcagaggaaaatgtctccgtgaacaacaaccacacccagccagtatgccttcacttcgccggcttctgccttttcttgctgcaggctccgccgctctggcaagccaagatacgtttcaaggcaagtgtactggttttgcagacaagataaacctgcctaatgtgcgggtaaattttgtcaattacgtgcctggaggcaccaatctttctttgccagataatcccaccagctgcggcacaacctctcaagtagtgtccgaggatgtctgccgtattgccatggctgttgcaacctcaaacagtagcgaaatcacccttgaagcatggctcccacaaaactacactggtcgtttcctgagtacgggcaacggtggtctctcaggctgtatgttctacccggcaccgcgatgcgacatggcacaacttcaaactaacgtcttacaggtattcagtactatgatctagcgtacacctccggcctcgggtttgccacggttggcgccaacagcggccataacggaacatccggggagcctttctaccaccacccagaggtcctcgaagactttgtacatcgttcagtccacactggtgtcgtggttggaaagcaattgacaaagcttttctacgaggaagggttcaagaagtcgtactaccttggttgctccactggtggtcggcagggctttaaatccgtccagaaatatcccaatgactttgatggtgttgtagccggtgcaccggcattcaatatgatcaacctcatgtcatggagtgcccacttctattcaatcacggggccagttgggtccgacacatacctatcccctgacctgtggaatatcacccataaggagatcctgcgtcaatgcgacggtatcgatggagcagaggacggcattattgaagacccaagtctttgcagcccggttcttgaagcgatcatctgcaagcctggtcaaaacactaccgagtgtttaactggcaagcaagcccataccgttcgcgaaattttctccccgctgtacggagtgaacggcaccttgctttatccccgcatgcagcctggctctgaggtgatggcttcttccataatgtacaacggccagcctttccagtatagcgcagactggtaccgctatgttgtctacgagaaccccaactgggatgcaaccaagttctccgtccgtgacgcagccgtcgctttgaagcagaacccattcaatctccagacctgggacgcagatatctcctctttccgcaaggcaggcggtaaagtcctcacctaccacggtctcatggatcaacttatcagctcggagaactccaagctttactatgcgcgcgttgcggaaaccatgaacgtccctccggaagagctggacgagttctaccgcttctttcagatcagtggaatggcccattgcagtggaggtgacggagcgtacggcattggaaaccagctcgtgacctataacgatgccaatcctgaaaacaacgtcctcatggctatggttcagtgggtggagaagggcatcgccccggagaccattcgtggtgctaagtttaccaatggcacgggctcggccgtggagtatactcgcaagcactgccgctaccctcgcaggaatgtatacaaggggccagggaactacactgatgagaatgcctggcaatgtgtttaaattgttgaagtattgtacatatatttgctcatagaggcaagacgtttgcatgtcttgataattatttattcgcccatcatagcagatagaatataagaccacgtcctacgaaactcgcagtgcacttgtataatt

SEQ ID NO: 2 - protein, 526 aa, Aspergillus oryzae
MPSLRRLLPFLAAGSAALASQDTFQGKCTGFADKINLPNVRVNFVNYVPGGTNLSLPDNPTSCGTTSQVVSEDVCRIAMAVATSNSSEITLEAWLPQNYTGRFLSTGNGGLSGCIQYYDLAYTSGLGFATVGANSGHNGTSGEPFYHHPEVLEDFVHRSVHTGVVVGKQLTKLFYEEGFKKSYYLGCSTGGRQGFKSVQKYPNDFDGVVAGAPAFNMINLMSWSAHFYSITGPVGSDTYLSPDLWNITHKEILRQCDGIDGAEDGIIEDPSLCSPVLEAIICKPGQNTTECLTGKQAHTVREIFSPLYGVNGTLLYPRMQPGSEVMASSIMYNGQPFQYSADWYRYVVYENPNWDATKFSVRDAAVALKQNPFNLQTWDADISSFRKAGGKVLTYHGLMDQLISSENSKLYYARVAETMNVPPEELDEFYRFFQISGMAHCSGGDGAYGIGNQLVTYNDANPENNVLMAMVQWVEKGIAPETIRGAKFTNGTGSAVEYTRKHCRYPRRNVYKGPGNYTDENAWQCV

SEQ ID NO: 3 - DNA, 1767 bp, Aspergillus oryzae
atgcgccaacactcgcgcatggccgttgctgctttggcagcaggagcgaacgcagcttcttttaccgatgtgtgcaccgtgtctaacgtgaaggctgcattgcctgccaacggaactctgctcggaatcagcatgcttccgtccgccgtcacggccaaccctctctacaaccagtcggctggcatgggtagcaccactacctatgactactgcaatgtgactgtcgcctacacgcataccggcaagggtgataaagtggtcatcaagtacgcattccccaagccctccgactacgagaaccgtttctacgttgctggtggtggtggcttttccctctctagcgatgctaccggaggtctcgcctatggcgctgtgggaggtgccaccgatgctggatacgacgcattcgataacagctacgacgaggtagtcctctacggaaacggaaccattaactgggacgccacatacatgttcgcataccaggcactgggagagatgacccggatcggaaagtacatcaccaagggcttttatggccagtccagcgacagcaaggtctacacctactacgagggttgctccgatggaggacgtgagggtatgagtcaagtccagcgctggggtgaggagtatgacggtgcgattactggtgccccggctttccgtttcgctcagcaacaggttcaccatgtgttctcgtccgaagtggagcaaactctggactactacccgcctccatgtgagttgaagaagatcgtgaacgccaccattgctgcttgcgacccgcttgatggaagaaccgacggtgttgtgtcccggacggatctttgcaagcttaacttcaatttgacctctatcatcggtgagccttactactgtgctgcgggaactagcacttcgcttggtttcggcttcagcaatggcaagcgcagcaatgtcaagcgtcaggccgagggcagcaccaccagctaccagcccgcccagaacggcacggtcaccgcacgtggtgtagctgtcgcccaggccatctacgatggtctccacaacagcaagggcgagcgcgcgtacctctcctggcagattgcctctgagctgagcgatgctgagaccgagtacaactctgacactggcaagtgggagctcaacatcccgtcgaccggtggtgagtacgtcaccaagttcattcagctcctgaacctcgacaacctttcggatctgaacaacgtgacctacgacaccctggtcgactggatgaacactggtatggtgcgctacatggacagccttcagaccacccttcccgatctgactcccttccaatcgtccggcggaaagctgctgcactaccacggtgaatctgaccccagtatccccgctgcctcctcggtccactactggcaggcggttcgttccgtcatgtacggcgacaagacggaagaggaggccctggaggctctcgaggactggtaccagttctacctaatccccggtgccgcccactgcggaaccaactctctccagcccggaccttaccctgagaacaacatggagattatgatcgactgggtcgagaacggcaacaagccgtcccgtctcaatgccactgtttcttcgggtacctacgccggcgagacccagatgctttgccagtggcccaagcgtcctctctggcgcggcaactccagcttcgactgtgtcaacgacgagaagtcgattgacagctggacctacgagttcccagccttcaaggtccctgtatactag

SEQ ID NO: 4 - protein, 588 aa, Aspergillus oryzae
MRQHSRMAVAALAAGANAASFTDVCTVSNVKAALPANGTLLGISMLPSAVTANPLYNQSAGMGSTTTYDYCNVTVAYTHTGKGDKVVIKYAFPKPSDYENRFYVAGGGGFSLSSDATGGLAYGAVGGATDAGYDAFDNSYDEVVLYGNGTINWDATYMFAYQALGEMTRIGKYITKGFYGQSSDSKVYTYYEGCSDGGREGMSQVQRWGEEYDGAITGAPAFRFAQQQVHHVFSSEVEQTLDYYPPPCELKKIVNATIAACDPLDGRTDGVVSRTDLCKLNFNLTSIIGEPYYCAAGTSTSLGFGFSNGKRSNVKRQAEGSTTSYQPAQNGTVTARGVAVAQAIYDGLHNSKGERAYLSWQIASELSDAETEYNSDTGKWELNIPSTGGEYVTKFIQLLNLDNLSDLNNVTYDTLVDWMNTGMVRYMDSLQTTLPDLTPFQSSGGKLLHYHGESDPSIPAASSVHYWQAVRSVMYGDKTEEEALEALEDWYQFYLIPGAAHCGTNSLQPGPYPENNMEIMIDWVENGNKPSRLNATVSSGTYAGETQMLCQWPKRPLWRGNSSFDCVNDEKSIDSWTYEFPAFKVPVY

SEQ ID NO: 5 - DNA, 1764 bp, Arxula adeninivorans
atggcaagcataccattctttgttgagatgaagcattttctcggacaatctttattgacaagtctgcttgcggcaggagcctttggatcctcgcttgccgaagtctgtacttcctcccgcatccggaccgccttaccaaaggatggagccatcgcagggatctctatggacccagacagtatcactgccaatccagtgtataatgcatctgctggctatagcgtgttttaccccgagggaaactttgattactgcaatgtgactgtttcctactgtcatattggcaagggtgacaaagtcaatctgcagtattggcttcctagtccagacaagttccaaaaccgttacctggctacaggcggcgggggatatgccatcaactctggaactcagtcactgcctggaggggtcatgtatggagcagttgctggtagaaccgatggaggatttggagggtttgatgtccaagtttctgaagccatcttgtacgccaatggatctctcaattacgatagtctatacatgtttggatatcgagcaattggtgagcagaccatgattggccaggagttagcgcgaggattctgtgaattgggggacgagaagaagatttacacatactaccaggggtgttcggaaggagtacgtgaaggctggagtcaaatcctaaaatttccagatctctacgatggagtaatccctgctgcccctgccttcagatatgggcatcagcaagtgaaccacctgtttccaggggtcatagaacaaggcatgaactattaccctccaccttgtgaaatggctcgtatcgtcaatgccacaattgaggcttgcgacaagctggatggcaagatagacggagtagtgtccaggacagatctgtgtctgttgaactttgactttaattctacaattgggctccattacacttgcgaagcaggctccaaccctatgacgggagactccaccccagcacaaaacggtactgtttccaccaaggctgctgagcttgctcgggtgttgacagaagggctccatgattcacaaggcaacaaggcatacgtcttttatcagattaccgccgggtatgacgatgcagacaccaagtacaaccctgccaccgggcagtttgaattgtcagtgagcagtcttggtggtgagtgggttacaaagctcttgcagcttgtcgaccttgacaatctaccaaaccttgacaatgttactgtggacacgctggttgattggatgcaatgcggttggcaaacttacgaagatgtgttacagacaaccaggcctgatctttctctgtatgaaagagccggaggaaagatcttgacattccacggggagtctgacaacagcatccctgcaggatcatcagtacatttttacgagtcagtgagaaacgtaatgtaccctggaatctcgtttaatcaaagcacagatgccatgggcgagtggtacaggctctatcttgtccccggagctgcccattgcagtatcaacgctttacaacccaatggtccattcccacaaaccacccttgaagtaatgattgactgggtagaaaatggcaatactccaaccacccttcaggctacatacttggttggtgacaataagggcaaaccagctgagatttgtccatggcccctgcgcccaacttggactgatgaaggaagcaagttacaatgcgtttatgatcatacctcgatcaatacctggatgtatgattttaacgctttttctctacccgtctactaa

SEQ ID NO: 6 - protein, 587 aa, Arxula adeninivorans
MASIPFFVEMKHFLGGSLLTSLLAAGAFGSSLAEVCTSSRIRTALPKDGAIAGISMDPDSITANPVYNASAGYSVFYPEGNFDYCNVTVSYCHIGKGDKVNLQYWLPSPDKFQNRYLATGGGGYAINSGTQSLPGGVMYGAVAGRTDGGFGGFDVQVSEAILYANGSLNYDSLYMFGYRAIGEQTMIGQELARGFCELGDEKKIYTYYQGCSEGVREGWSQILKFPDLYDGVIPAAPAFRYGHQQVNHLFPGVIEQGMNYYPPPCEMARIVNATIEACDKLDGKIDGVVSRTDLCLLNFDFNSTIGLHYTCEAGSNPMTGDSTPAQNGTVSTKAAELARVLTEGLHDSQGNKAYVFYQITAGYDDADTKYNPATGQFELSVSSLGGEWVTKLLQLVDLDNLPNLDNVTVDTLVDWMQCGWQTYEDVLQTTRPDLSLYERAGGKILTFHGESDNSIPAGSSVHFYESVRNVMYPGISFNQSTDAMGEWYRLYLVPGAAHCSINALQPNGPFPQTTLEVMIDWVENGNTPTTLQATYLVGDNKGKPAEICPWPLRPTWTDEGSKLQCVYDHTSINTWMYDFNAFSLPVY

SEQ ID NO: 7 - DNA, 1842 bp, Staphylococcus lugdunensis
atgaaaaagactttcatatcactcttatccgcaacagttatactttcaggttgtggcgttggcgaacatcaaaataataattctaatcatgatgctaaaggtgtgaacacttcaaatgttaaaatcaaaaattataaccaagcatcatctgcgctgcaaatagataattcaaaatggaaatatgatagtaaaaataacgtttattatcaactaaatataagttatgtctccaatccccaagctaaaaatgtagaaaaattaggtatctatgtaccagctgcttatttcaaaggtaaaaagaatcataatgggacatataccgttactgtaaacgatgctaagaaagttaacggctattctgctagaacagcacctatcgtttatccagtcaatacacctggttatgccgaacaaagtgcacctacgtcatatcgttatagtaatatttctaagtatatgaaagctggattcatatatgttgaagcaggattacgaggacgtagtatgagcatgggcaataacagcagtaatgcatcaactaaatcatatgaaaccggttctccttggggtgtaaccgatcttaaagcagcaatcagatattaccgtttcaacgatagtagtctaccaggtaacagtagtaagatttatacttttggtcatagtggcggtggtgctcaaagtgctattgccggtgcatcaggtgatagcaagctctactataaatatttagaacaaattggcgcagccatgacagataaaaatggaaaatatatcagtgataaaattgacggtgctatggcgtggtgccctattacaagtctagatcaagccgatgctgcttatgaatggcaaatgggacaatatggtaatgaaggtaatcgcaagaaaaattcattccaaaaacaattatcaaccgatttagcatcatcttatgcaagctacttaaataaactaaatctgaaaaatggaaatactacattatcattaactaaatctaaaaatggtcaatatactgaaggctcatatgctaaatatctaaaaaaagaaattgaagattcagctacagaattcttaaataatacaacattcccttacaaacaaaatagcactgagcaagcaggcatgggtaatggtggacctagcggtggaaaaccttctggcaaaatgggatctatgcctcaaatgagaaaacaatcttcaaataaaacatacaaaacaatggatgcttacttaaaagatctaaataaaaaaggcacatggatcacgtatgataagaaaacaaaacgcgcacatattacaagtcttaaagactttgcgaaatattataaacaaccttctaaatcagtttcagcctttgatgatttaaaacgtagccaagctgaaaatgaagtgtttggaacatcaggtagtgacagtaaattacattttgatcaatcactagctaaacttttaacagaaaataaatctaactatagcaaactaaatggttggaatagtaactatgtttcatcatataaaaatgacttaacaaaaacagataaattaggcacaagcatgtcaacaagaatgaatatgtacaatccaatgtattacttatctgattactatagcgggtatggtaaatctaatgtggcaaatcattggagaattagaacaggtattcaacaaggagatacggccttaaatactgaaactaatctttcgctagctttaaaagaacgcgttggttctaaaaacgttgacttcaaaacagtttgggatcaaggtcatacaatggcagaaacatcaggtaatagtgatagtaacttcatcaaatgggtagaaagtattaataaaaaatag

SEQ ID NO: 8 - protein, 613 aa, Staphylococcus lugdunensis
MKKTFISLLSATVILSGCGVGEHQNNNSNHDAKGVNTSNVKIKNYNQASSALQIDNSKWKYDSKNNVYYQLNISYVSNPQAKNVEKLGIYVPAAYFKGKKNHNGTYTVTVNDAKKVNGYSARTAPIVYPVNTPGYAEQSAPTSYRYSNISKYMKAGFIYVEAGLRGRSMSMGNNSSNASTKSYETGSPWGVTDLKAAIRYYRFNDSSLPGNSSKIYTFGHSGGGAQSAIAGASGDSKLYYKYLEQIGAAMTDKNGKYISDKIDGAMAWCPITSLDQADAAYEWQMGQYGNEGNRKKNSFQKQLSTDLASSYASYLNKLNLKNGNTTLSLTKSKNGQYTEGSYAKYLKKEIEDSATEFLNNTTFPYKQNSTEQAGMGNGGPSGGKPSGKMGSMPQMRKQSSNKTYKTMDAYLKDLNKKGTWITYDKKTKRAHITSLKDFAKYYKQPSKSVSAFDDLKRSQAENEVFGTSGSDSKLHFDQSLAKLLTENKSNYSKLNGWNSNYVSSYKNDLTKTDKLGTSMSTRMNMYNPMYYLSDYYSGYGKSNVANHWRIRTGIQQGDTALNTETNLSLALKERVGSKNVDFKTVWDQGHTMAETSGNSDSNFIKWVESINKK

SEQ ID NO: 9 - DNA, 1767 bp, Aspergillus niger
atgtacagcctggctgctgccactcttgtcggtgtcgcatctgcggcatcgctgaacagtgtgtgtacaaccgactatgtcacgtcggttctgcctactgccagcgatgacattccttctggaatcaccatcgacactagctctgtatctgctagtatctaccgcaactattccctcaccgattccattttctgggaggatttgaccatcaacttctgtgaagtatcttttgcctacagccaccagaacggagatgaccgcgtagtcgtccaatattggatgccgagcccagaccttttccagaacagattcctcgctacaggtggttccgcgtatgagatcaacaacggctcaggaggaggtgatatcgccggaggggtcgcctttggggctgccactggctacaccgacggtggattcccttactggggtggcactgacttcgatgatgttgtcattctcggcaatggaactgccaactggcctgccatatacaactggggataccaggccattgccgaaatgacccagattggaaaggcctttaccaacaacttcttcaacgtcggaaataacgttaccaagttgtacacctattacatcggttgctctgaaggtggacgtgagggaatgagccaagcccaacgtgcccccgaattgtacgatggcatcgttgctggtgcccctgctatgcgctacggccagcagcaggtgaatcacatcgctcctcccatccagatccagactatcggctattatccgccttcttgcgtgtttgatacagtgatcaacgcaacgatcaatgcctgtgatggcatggacggcaagattgatggagtggttgctcgtagcgatctctgtttccagaatttcaatgtatcctcaatgctgggcaagtcgtactactgcgaggctgggtcgaccactagccttggcttgggatatgggaagcggagcaagaggcaaacaacttcagccacccctgcgcaaaatggaaccattaatgccaaagatattgaggtgattcaagaccttctaactggactgaaagactcaaacggtgacctcgtgtatttccctttccagcctactgccggctttggcgacactactgtctacgacagcaccacggattcctggacgatcacatctcccaactccaacggagaatggattaccaaattcctaaattggcagaacgtcacggatttggacatgtggggagtcaccaatgatgacctgaaggcatggatgatcgaaggaatgaccaaatacatggactctcttcaaaccactcttcctgacctgacccccttccattccaagggaggccgtctgcttcattaccatggagaggccgatagcagtgttcccccgaccggatccattcactaccacgaatcggttcgcgagatcatgtatcctgacctctcttttgctgagggcaatgagaaactcaacgactggtaccgtttctatctcgtccctggtgcagcccactgcgcaaccaacgatgagcaacccaatgctggtttccctcgggacaatttcgcccacatgatcaagtgggtagaggaagacgtagtacctgtcagaatcaatgccactgttacttctggggagcacaagggcgaagtccaggagctttgcacttggccgtcgcgcccatactggactgacaacaacactatggtctgcgaacagaacgcaacctctatccaggccatgctctggaagttgagcgcctaccttacgcctgtctactag

SEQ ID NO: 10 - protein, 588 aa, Aspergillus niger
MYSLAAATLVGVASAASLNSVCTTDYVTSVLPTASDDIPSGITIDTSSVSASIYRNYSLTDSIFWEDLTINFCEVSFAYSHQNGDDRVVVQYWMPSPDLFQNRFLATGGSAYEINNGSGGGDIAGGVAFGAATGYTDGGFPYWGGTDFDDVVILGNGTANWPAIYNWGYQAIAEMTQIGKAFTNNFFNVGNNVTKLYTYYIGCSEGGREGMSQAQRAPELYDGIVAGAPAMRYGQQQVNHIAPPIQIQTIGYYPPSCVFDTVINATINACDGMDGKIDGVVARSDLCFQNFNVSSMLGKSYYCEAGSTTSLGLGYGKRSKRQTTSATPAQNGTINAKDIEVIQDLLTGLKDSNGDLVYFPFQPTAGFGDTTVYDSTTDSWTITSPNSNGEWITKFLNWQNVTDLDMWGVTNDDLKAWMIEGMTKYMDSLQTTLPDLTPFHSKGGRLLHYHGEADSSVPPTGSIHYHESVREIMYPDLSFAEGNEKLNDWYRFYLVPGAAHCATNDEQPNAGFPRDNFAHMIKWVEEDVVPVRINATVTSGEHKGEVQELCTWPSRPYWTDNNTMVCEQNATSIQAMLWKLSAYLTPVY

SEQ ID NO: 11 - DNA, 923 bp, Humicola insolens
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggcggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatcccctccagcagcaccagctctccggtcaaccagcctaccagcaccagcaccacgtccacctccaccacctcgagcccgccagtccagcctacgactcccagcggctgcactgctgagaggtgggctcagtgcggcggcaatggctggagcggctgcaccacctgcgtcgctggcagcacttgcacgaagattaatgactggtaccatcagtgcctgtagaattc

SEQ ID NO: 12 - protein, 305 aa, Humicola insolens
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPSSSTSSPVNQPTSTSTTSTSTTSSPPVQPTTPSGCTAERWAQCGGNGWSGCTTCVAGSTCTKINDWYHQCL

SEQ ID NO: 13 - DNA, 1188 bp, Myceliophthora thermophila
cgacttgaaacgccccaaatgaagtcctccatcctcgccagcgtcttcgccacgggcgccgtggctcaaagtggtccgtggcagcaatgtggtggcatcggatggcaaggatcgaccgactgtgtgtcgggctaccactgcgtctaccagaacgattggtacagccagtgcgtgcctggcgcggcgtcgacaacgctgcagacatcgaccacgtccaggcccaccgccaccagcaccgcccctccgtcgtccaccacctcgcctagcaagggcaagctgaagtggctcggcagcaacgagtcgggcgccgagttcggggagggcaattaccccggcctctggggcaagcacttcatcttcccgtcgacttcggcgattcagacgctcatcaatgatggatacaacatcttccggatcgacttctcgatggagcgtctggtgcccaaccagttgacgtcgtccttcgaccagggttacctccgcaacctgaccgaggtggtcaacttcgtgacgaacgcgggcaagtacgccgtcctggacccgcacaactacggccggtactacggcaacatcatcacggacacgaacgcgttccggaccttctggaccaacctggccaagcagttcgcctccaactcgctcgtcatcttcgacaccaacaacgagtacaacacgatggaccagaccctggtgctcaacctcaaccaggccgccatcgacggcatccgggccgccggcgcgacctcgcagtacatcttcgtcgagggcaacgcgtggagcggggcctggagctggaacacgaccaacaccaacatggccgccctgacggacccgcagaacaagatcgtgtacgagatgcaccagtacctcgactcggacagctcgggcacccacgccgagtgcgtcagcagcaccatcggcgcccagcgcgtcgtcggagccacccagtggctccgcgccaacggcaagctcggcgtcctcggcgagttcgccggcggcgccaacgccgtctgccagcaggccgtcaccggcctcctcgaccacctccaggacaacagcgacgtctggctgggtgccctctggtgggccgccggtccctggtggggcgactacatgtactcgttcgagcctccttcgggcaccggctatgtcaactacaactcgatcttgaagaagtacttgccgtaa

SEQ ID NO: 14 - protein, 389 aa, Myceliophthora thermophila
MKSSILASVFATGAVAQSGPWQQCGGIGWQGSTDCVSGYHCVYQNDWYSQCVPGAASTTLQTSTTSRPTATSTAPPSSTTSPSKGKLKWLGSNESGAEFGEGNYPGLWGKHFIFPSTSAIQTLINDGYNIFRIDFSMERLVPNQLTSSFDQGYLRNLTEVVNFVTNAGKYAVLDPHNYGRYYGNIITDTNAFRTFWTNLAKQFASNSLVIFDTNNEYNTMDQTLVLNLNQAAIDGIRAAGATSQYIFVEGNAWSGAWSWNTTNTNMAALTDPQNKIVYEMHQYLDSDSSGTHAECVSSTIGAQRVVGATQWLRANGKLGVLGEFAGGANAVCQQAVTGLLDHLQDNSDVWLGALWWAAGPWWGDYMYSFEPPSGTGYVNYNSILKKYLP

SEQ ID NO: 15 - DNA, 1232 bp, Basidiomycete CBS 495.95
ggatccacttagtaacggccgccagtgtgctggaaagcatgaagtctctcttcctgtcacttgtagcgaccgtcgcgctcagctcgccagtattctctgtcgcagtctgggggcaatgcggcggcattggcttcagcggaagcaccgtctgtgatgcaggcgccggctgtgtgaagctcaacgactattactctcaatgccaacccggcgctcccactgctacatccgcggcgccaagtagcaacgcaccgtccggcacttcgacggcctcggccccctcctccagcctttgctctggcagccgcacgccgttccagttcttcggtgtcaacgaatccggcgcggagttcggcaacctgaacatccccggtgttctgggcaccgactacacctggccgtcgccatccagcattgacttcttcatgggcaagggaatgaataccttccgtattccgttcctcatggagcgtcttgtcccccctgccactggcatcacaggacctctcgaccagacgtacttgggcggcctgcagacgattgtcaactacatcaccggcaaaggcggctttgctctcattgacccgcacaactttatgatctacaatggccagacgatctccagtaccagcgacttccagaagttctggcagaacctcgcaggagtgtttaaatcgaacagtcacgtcatcttcgatgttatgaacgagcctcacgatattcccgcccagaccgtgttccaactgaaccaagccgctgtcaatggcatccgtgcgagcggtgcgacgtcgcagctcattctggtcgagggcacaagctggactggagcctggacctggacgacctctggcaacagcgatgcattcggtgccattaaggatcccaacaacaacgtcgcgatccagatgcatcagtacctggatagcgatggctctggcacttcgcagacctgcgtgtctcccaccatcggtgccgagcggttgcaggctgcgactcaatggttgaagcagaacaacctcaagggcttcctgggcgagatcggcgccggctctaactccgcttgcatcagcgctgtgcagggtgcgttgtgttcgatgcagcaatctggtgtgtggctcggcgctctctggtgggctgcgggcccgtggtggggcgactactaccagtccatcgagccgccctctggcccggcggtgtccgcgatcctcccgcaggccctgctgccgttcgcgtaa

SEQ ID NO: 16 - protein, 397 aa, Basidiomycete CBS 495.95
MKSLFLSLVATVALSSPVFSVAVWGQCGGIGFSGSTVCDAGAGCVKLNDYYSQCQPGAPTATSAAPSSNAPSGTSTASAPSSSLCSGSRTPFQFFGVNESGAEFGNLNIPGVLGTDYTWPSPSSIDFFMGKGMNTFRIPFLMERLVPPATGITGPLDQTYLGGLQTIVNYITGKGGFALIDPHNFMIYNGQTISSTSDFQKFWQNLAGVFKSNSHVIFDVMNEPHDIPAQTVFQLNQAAVNGIRASGATSQLILVEGTSWTGAWTWTTSGNSDAFGAIKDPNNNVAIQMHQYLDSDGSGTSQTCVSPTIGAERLQAATQWLKQNNLKGFLGEIGAGSNSACISAVQGALCSMQQSGVWLGALWWAAGPWWGDYYQSIEPPSGPAVSAILPQALLPFA

SEQ ID NO: 17 - DNA, 1303 bp, Basidiomycete CBS 494.95
ggaaagcgtcagtatggtgaaatttgcgcttgtggcaactgtcggcgcaatcttgagcgcttctgcggccaatgcggcttctatctaccagcaatgtggaggcattggatggtctgggtccactgtttgcgacgccggtctcgcttgcgttatcctcaatgcgtactactttcagtgcttgacgcccgccgcgggccagacaacgacgggctcgggcgcaccggcgtcaacatcaacctctcactcaacggtcactacggggagctcacactcaacaaccgggacgacggcgacgaaaacaactaccactccgtcgaccaccacgaccctacccgccatctctgtgtctggtcgcgtctgctctggctccaggacgaagttcaagttcttcggtgtgaatgaaagcggcgccgaattcgggaacactgcttggccagggcagctcgggaaagactatacatggccttcgcctagcagcgtggactacttcatgggggctggattcaatacattccgtatcaccttcttgatggagcgtatgagccctccggctaccggactcactggcccattcaaccagacgtacctgtcgggcctcaccaccattgtcgactacatcacgaacaaaggaggatacgctcttattgacccccacaacttcatgcgttacaacaacggcataatcagcagcacatctgacttcgcgacttggtggagcaatttggccactgtattcaaatccacgaagaacgccatcttcgacatccagaacgagccgtacggaatcgatgcgcagaccgtatacgaactgaatcaagctgccatcaattcgatccgcgccgctggcgctacgtcacagttgattctggttgaaggaacgtcatacactggagcttggacgtgggtctcgtccggaaacggagctgctttcgcggccgttacggatccttacaacaacacggcaattgaaatgcaccaatacctcgacagcgacggttctgggacaaacgaagactgtgtctcctccaccattgggtcgcaacgtctccaagctgccactgcgtggctgcaacaaacaggactcaagggattcctcggagagacgggtgctgggtcgaattcccagtgcatcgacgccgtgttcgatgaactttgctatatgcaacagcaaggcggctcctggatcggtgcactctggtgggctgcgggtccctggtggggcacgtacatttactcgattgaacctccgagcggtgccgctatcccagaagtccttcctcagggtctcgctccattcctctag

SEQ ID NO: 18 - protein, 429 aa, Basidiomycete CBS 494.95
MVKFALVATVGAILSASAANAASIYQQCGGIGWSGSTVCDAGLACVILNAYYFQCLTPAAGQTTTGSGAPASTSTSHSTVTTGSSHSTTGTTATKTTTTPSTTTTLPAISVSGRVCSGSRTKFKFFGVNESGAEFGNTAWPGQLGKDYTWPSPSSVDYFMGAGFNTFRITFLMERMSPPATGLTGPFNQTYLSGLTTIVDYITNKGGYALIDPHNFMRYNNGIISSTSDFATWWSNLATVFKSTKNAIFDIQNEPYGIDAQTVYELNQAAINSIRAAGATSQLILVEGTSYTGAWTWVSSGNGAAFAAVTDPYNNTAIEMHQYLDSDGSGTNEDCVSSTIGSQRLQAATAWLQQTGLKGFLGETGAGSNSQCIDAVFDELCYMQQQGGSWIGALWWAAGPWWGTYIYSIEPPSGAAIPEVLPQGLAPFL

SEQ ID NO: 19 - DNA, 1580 bp, Thielavia terrestris
agccccccgttcaggcacacttggcatcagatcagcttagcagcgcctgcacagcatgaagctctcgcagtcggccgcgctggcggcactcaccgcgacggcgctcgccgccccctcgcccacgacgccgcaggcgccgaggcaggcttcagccggctgctcgtctgcggtcacgctcgacgccagcaccaacgtttggaagaagtacacgctgcaccccaacagctactaccgcaaggaggttgaggccgcggtggcgcagatctcggacccggacctcgccgccaaggccaagaaggtggccgacgtcggcaccttcctgtggctcgactcgatcgagaacatcggcaagctggagccggcgatccaggacgtgccctgcgagaacatcctgggcctggtcatctacgacctgccgggccgcgactgcgcggccaaggcgtccaacggcgagctcaaggtcggcgagatcgaccgctacaagaccgagtacatcgacagtgagtgctgccccccgggttcgagaagagcgtgggggaaagggaaagggttgactgactgacacggcgcactgcagagatcgtgtcgatcctcaaggcacaccccaacacggcgttcgcgctggtcatcgagccggactcgctgcccaacctggtgaccaacagcaacttggacacgtgctcgagcagcgcgtcgggctaccgcgaaggcgtggcttacgccctcaagaacctcaacctgcccaacgtgatcatgtacctcgacgccggccacggcggctggctcggctgggacgccaacctgcagcccggcgcgcaggagctagccaaggcgtacaagaacgccggctcgcccaagcagctccgcggcttctcgaccaacgtggccggctggaactcctggtgagcttttttccattccatttcttcttcctcttctctcttcgctcccactctgcagccccccctcccccaagcacccactggcgttccggcttgctgactcggcctccctttccccgggcaccagggatcaatcgcccggcgaattctcccaggcgtccgacgccaagtacaacaagtgccagaacgagaagatctacgtcagcaccttcggctccgcgctccagtcggccggcatgcccaaccacgccatcgtcgacacgggccgcaacggcgtcaccggcctgcgcaaggagtggggtgactggtgcaacgtcaacggtgcaggttcgttgtcttctttttctcctcttttgtttgcacgtcgtggtccttttcaagcagccgtgtttggttgggggagatggactccggctgatgttctgcttcctctctaggcttcggcgtgcgcccgacgagcaacacgggcctcgagctggccgacgcgttcgtgtgggtcaagcccggcggcgagtcggacggcaccagcgacagctcgtcgccgcgctacgacagcttctgcggcaaggacgacgccttcaagccctcgcccgaggccggcacctggaacgaggcctacttcgagatgctgctcaagaacgccgtgccgtcgttctaagacggtccagcatcatccgg

SEQ ID NO: 20 - protein, 396 aa, Thielavia terrestris
MKLSQSAALAALTATALAAPSPTTPQAPRQASAGCSSAVTLDASTNVWKKYTLHPNSYYRKEVEAAVAQISDPDLAAKAKKVADVGTFLWLDSIENIGKLEPAIQDVPCENILGLVIYDLPGRDCAAKASNGELKVGEIDRYKTEYIDKIVSILKAHPNTAFALVIEPDSLPNLVTNSNLDTCSSSASGYREGVAYALKNLNLPNVIMYLDAGHGGWLGWDANLQPGAQELAKAYKNAGSPKQLRGFSTNVAGWNSWDQSPGEFSQASDAKYNKCQNEKIYVSTFGSALQSAGMPNHAIVDTGRNGVTGLRKEWGDWCNVNGAGFGVRPTSNTGLELADAFVWVKPGGESDGTSDSSSPRYDSFCGKDDAFKPSPEAGTWNEAYFEMLLKNAVPSF

SEQ ID NO: 21 - DNA, 1203 bp, Thielavia terrestris
atgaagtacctcaacctcctcgcagctctcctcgccgtcgctcctctctccctcgctgcacccagcatcgaggccagacagtcgaacgtcaacccatacatcggcaagagcccgctcgttattaggtcgtacgcccaaaagcttgaggagaccgtcaggaccttccagcaacgtggcgaccagctcaacgctgcgaggacacggacggtgcagaacgttgcgactttcgcctggatctcggataccaatggtattggagccattcgacctctcatccaagatgctctcgcccagcaggctcgcactggacagaaggtcatcgtccaaatcgtcgtctacaacctcccagatcgcgactgctctgccaacgcctcgactggagagttcaccgtaggaaacgacggtctcaaccgatacaagaactttgtcaacaccatcgcccgcgagctctcgactgctgacgctgacaagctccactttgccctcctcctcgaacccgacgcacttgccaacctcgtcaccaacgcgaatgcccccaggtgccgaatcgccgctcccgcttacaaggagggtatcgcctacaccctcgccaccttgtccaagcccaacgtcgacgtctacatcgacgccgccaacggtggctggctcggctggaacgacaacctccgccccttcgccgaactcttcaaggaagtctacgacctcgcccgccgcatcaaccccaacgccaaggtccgcggcgtccccgtcaacgtctccaactacaaccagtaccgcgctgaagtccgcgagcccttcaccgagtggaaggacgcctgggacgagagccgctacgtcaacgtcctcaccccgcacctcaacgccgtcggcttctccgcgcacttcatcgttgaccagggacgcggtggcaagggcggtatcaggacggagtggggccagtggtgcaacgttaggaacgctgggttcggtatcaggcctactgcggatcagggcgtgctccagaacccgaatgtggatgcgattgtgtgggttaagccgggtggagagtcggatggcacgagtgatttgaactcgaacaggtatgatcctacgtgcaggagtccggtggcgcatgttcccgctcctgaggctggccagtggttcaacgagtatgttgttaacctcgttttgaacgctaacccccctcttgagcctacctggtaa

SEQ ID NO: 22 - protein, 400 aa, Thielavia terrestris
MKYLNLLAALLAVAPLSLAAPSIEARQSNVNPYIGKSPLVIRSYAQKLEETVRTFQQRGDQLNAARTRTVQNVATFAWISDTNGIGAIRPLIQDALAQQARTGQKVIVQIVVYNLPDRDCSANASTGEFTVGNDGLNRYKNFVNTIARELSTADADKLHFALLLEPDALANLVTNANAPRCRIAAPAYKEGIAYTLATLSKPNVDVYIDAANGGWLGWNDNLRPFAELFKEVYDLARRINPNAKVRGVPVNVSNYNQYRAEVREPFTEWKDAWDESRYVNVLTPHLNAVGFSAHFIVDQGRGGKGGIRTEWGQWCNVRNAGFGIRPTADQGVLQNPNVDAIVWVKPGGESDGTSDLNSNRYDPTCRSPVAHVPAPEAGQWFNEYVVNLVLNANPPLEPTW

SEQ ID NO: 23 - DNA, 1501 bp, Thielavia terrestris
gccgttgtcaagatgggccagaagacgctgcacggattcgccgccacggctttggccgttctcccctttgtgaaggctcagcagcccggcaacttcacgccggaggtgcacccgcaactgccaacgtggaagtgcacgaccgccggcggctgcgttcagcaggacacttcggtggtgctcgactggaactaccgttggatccacaatgccgacggcaccgcctcgtgcacgacgtccagcggggtcgaccacacgctgtgtccagatgaggcgacctgcgcgaagaactgcttcgtggaaggcgtcaactacacgagcagcggtgtcaccacatccggcagttcgctgacgatgaggcagtatttcaaggggagcaacgggcagaccaacagcgtttcgcctcgtctctacctgctcggctcggatggaaactacgtaatgctcaagctgctcggccaggagctgagcttcgatgtcgatctctccacgctcccctgcggcgagaacggcgcgctgtacctgtccgagatggacgcgaccggtggcaggaaccagtacaacaccggcggtgccaactacggctcgggctactgtgacgcccagtgtcccgtgcagacgtggatgaacggcacgctgaacaccaacgggcagggctactgctgcaacgagatggacatcctcgaggccaactcccgcgccaacgcgatgacacctcacccctgcgccaacggcagctgcgacaagagcgggtgcggactcaacccctacgccgagggctacaagagctactacggaccgggcctcacggttgacacgtcgaagcccttcaccatcattacccgcttcatcaccgacgacggcacgaccagcggcaccctcaaccagatccagcggatctatgtgcagaatggcaagacggtcgcgtcggctgcgtccggaggcgacatcatcacggcatccggctgcacctcggcccaggcgttcggcgggctggccaacatgggcgcggcgcttggacggggcatggtgctgaccttcagcatctggaacgacgctgggggctacatgaactggctcgacagcggcaacaacggcccgtgcagcagcaccgagggcaacccgtccaacatcctggccaactacccggacacccacgtggtcttctccaacatccgctggggagacatcggctcgacggtccaggtctcgggaggcggcaacggcggctcgaccaccaccacgtcgaccaccacgctgaggacctcgaccacgaccaccaccaccgccccgacggccactgccacgcactggggacaatgcggcggaatcggggtacgtcaaccgcctcctgcattctgttgaggaagttaactaacgtggcctacgcagtggactggaccgaccgtctgcgaatcgccgtacgcatgcaaggagctgaacccctggtactaccagtgcctctaaagtattgcagtgaagccatactccgtgctcggcatgg

SEQ ID NO: 24 - protein, 464 aa, Thielavia terrestris
MGQKTLHGFAATALAVLPFVKAQQPGNFTPEVHPQLPTWKCTTAGGCVQQDTSVVLDWNYRWIHNADGTASCTTSSGVDHTLCPDEATCAKNCFVEGVNYTSSGVTTSGSSLTMRQYFKGSNGQTNSVSPRLYLLGSDGNYVMLKLLGQELSFDVDLSTLPCGENGALYLSEMDATGGRNQYNTGGANYGSGYCDAQCPVQTWMNGTLNTNGQGYCCNEMDILEANSRANAMTPHPCANGSCDKSGCGLNPYAEGYKSYYGPGLTVDTSKPFTIITRFITDDGTTSGTLNQIQRIYVQNGKTVASAASGGDIITASGCTSAQAFGGLANMGAALGRGMVLTFSIWNDAGGYMNWLDSGNNGPCSSTEGNPSNILANYPDTHVVFSNIRWGDIGSTVQVSGGGNGGSTTTTSTTTLRTSTTTTTTAPTATATHWGQCGGIGWTGPTVCESPYACKELNPWYYQCL

SEQ ID NO: 25 - DNA, 1368 bp, Thielavia terrestris
accgatccgctcgaagatggcgcccaagtctacagttctggccgcctggctgctctcctcgctggccgcggcccagcagatcggcaaagccgtgcccgaggtccaccccaaactgacaacgcagaagtgcactctccgcggcgggtgcaagcctgtccgcacctcggtcgtgctcgactcgtccgcgcgctcgctgcacaaggtcggggaccccaacaccagctgcagcgtcggcggcgacctgtgctcggacgcgaagtcgtgcggcaagaactgcgcgctcgagggcgtcgactacgcggcccacggcgtggcgaccaagggcgacgccctcacgctgcaccagtggctcaagggggccgacggcacctacaggaccgtctcgccgcgcgtatacctcctgggcgaggacgggaagaactacgaggacttcaagctgctcaacgccgagctcagcttcgacgtcgacgtgtcccagctcgtctgcggcatgaacggcgccctgtacttctccgagatggagatggacggcggccgcagcccgctgaacccggcgggcgccacgtacggcacgggctactgcgacgcgcagtgccccaagttggactttatcaacggcgaggtatttcttctctcttctgtttttcttttccatcgctttttctgaccggaatccgccctcttagctcaacaccaaccacacgtacggggcgtgctgcaacgagatggacatctgggaggccaacgcgctggcgcaggcgctcacgccgcacccgtgcaacgcgacgcgggtgtacaagtgcgacacggcggacgagtgcgggcagccggtgggcgtgtgcgacgaatgggggtgctcgtacaacccgtccaacttcggggtcaaggactactacgggcgcaacctgacggtggacacgaaccgcaagttcacggtgacgacgcagttcgtgacgtccaacgggcgggcggacggcgagctgaccgagatccggcggctgtacgtgcaggacggcgtggtgatccagaaccacgcggtcacggcgggcggggcgacgtacgacagcatcacggacggcttctgcaacgcgacggccacctggacgcagcagcggggcgggctcgcgcgcatgggcgaggccatcggccgcggcatggtgctcatcttcagcctgtgggttgacaacggcggcttcatgaactggctcgacagcggcaacgccgggccctgcaacgccaccgagggcgacccggccctgatcctgcagcagcacccggacgccagcgtcaccttctccaacatccgatggggcgagatcggcagcacgtacaagagcgagtgcagccactagagtagagcttgtaatt

SEQ ID NO: 26 - protein, 423 aa, Thielavia terrestris
MAPKSTVLAAWLLSSLAAAQQIGKAVPEVHPKLTTQKCTLRGGCKPVRTSVVLDSSARSLHKVGDPNTSCSVGGDLCSDAKSCGKNCALEGVDYAAHGVATKGDALTLHQWLKGADGTYRTVSPRVYLLGEDGKNYEDFKLLNAELSFDVDVSQLVCGMNGALYFSEMEMDGGRSPLNPAGATYGTGYCDAQCPKLDFINGELNTNHTYGACCNEMDIWEANALAQALTPHPCNATRVYKCDTADECGQPVGVCDEWGCSYNPSNFGVKDYYGRNLTVDTNRKFTVTTQFVTSNGRADGELTEIRRLYVQDGVVIQNHAVTAGGATYDSITDGFCNATATWTQQRGGLARMGEAIGRGMVLIFSLWVDNGGFMNWLDSGNAGPCNATEGDPALILQQHPDASVTFSNIRWGEIGSTYKSECSH

SEQ ID NO: 27 - DNA, 1011 bp, Thielavia terrestris
atgaccctacggctccctgtcatcagcctgctggcctcgctggcagcaggcgccgtcgtcgtcccacgggcggagtttcacccccctctcccgacttggaaatgcacgacctccgggggctgcgtgcagcagaacaccagcgtcgtcctggaccgtgactcgaagtacgccgcacacagcgccggctcgcggacggaatcggattacgcggcaatgggagtgtccacttcgggcaatgccgtgacgctgtaccactacgtcaagaccaacggcaccctcgtccccgcttcgccgcgcatctacctcctgggcgcggacggcaagtacgtgcttatggacctcctcaaccaggagctgtcggtggacgtcgacttctcggcgctgccgtgcggcgagaacggggccttctacctgtccgagatggcggcggacgggcggggcgacgcgggggcgggcgacgggtactgcgacgcgcagtgccagggctactgctgcaacgagatggacatcctcgaggccaactcgatggcgacggccatgacgccgcacccgtgcaagggcaacaactgcgaccgcagcggctgcggctacaacccgtacgccagcggccagcgcggcttctacgggcccggcaagacggtcgacacgagcaagcccttcaccgtcgtcacgcagttcgccgccagcggcggcaagctgacccagatcacccgcaagtacatccagaacggccgggagatcggcggcggcggcaccatctccagctgcggctccgagtcttcgacgggcggcctgaccggcatgggcgaggcgctggggcgcggaatggtgctggccatgagcatctggaacgacgcggcccaggagatggcatggctcgatgccggcaacaacggcccttgcgccagtggccagggcagcccgtccgtcattcagtcgcagcatcccgacacccacgtcgtcttctccaacatcaggtggggcgacatcgggtctaccacgaagaactag

SEQ ID NO: 28 - protein, 336 aa, Thielavia terrestris
MTLRLPVISLLASLAAGAVVVPRAEFHPPLPTWKCTTSGGCVQQNTSVVLDRDSKYAAHSAGSRTESDYAAMGVSTSGNAVTLYHYVKTNGTLVPASPRIYLLGADGKYVLMDLLNQELSVDVDFSALPCGENGAFYLSEMAADGRGDAGAGDGYCDAQCQGYCCNEMDILEANSMATAMTPHPCKGNNCDRSGCGYNPYASGQRGFYGPGKTVDTSKPFTVVTQFAASGGKLTQITRKYIQNGREIGGGGTISSCGSESSTGGLTGMGEALGRGMVLAMSIWNDAAQEMAWLDAGNNGPCASGQGSPSVIQSQHPDTHVVFSNIRWGDIGSTTKN

SEQ ID NO: 29 - DNA, 1480 bp, Cladorrhinum foecundissimum
gatccgaattcctcctctcgttctttagtcacagaccagacatctgcccacgatggttcacaagttcgccctcctcaccggcctcgccgcctccctcgcatctgcccagcagatcggcaccgtcgtccccgagtctcaccccaagcttcccaccaagcgctgcactctcgccggtggctgccagaccgtcgacacctccatcgtcatcgacgccttccagcgtcccctccacaagatcggcgacccttccactccttgcgtcgtcggcggccctctctgccccgacgccaagtcctgcgctgagaactgcgcgctcgagggtgtcgactatgcctcctggggcatcaagaccgagggcgacgccctaactctcaaccagtggatgcccgacccggcgaaccctggccagtacaagacgactactccccgtacttaccttgttgctgaggacggcaagaactacgaggatgtgaagctcctggctaaggagatctcgtttgatgccgatgtcagcaaccttccctgcggcatgaacggtgctttctacttgtctgagatgttgatggatggtggacgtggcgacctcaaccctgctggtgccgagtatggtaccggttactgtgatgcgcagtgcttcaagttggatttcatcaacggcgaggccaacatcgaccaaaagcacggcgcctgctgcaacgaaatggacattttcgaatccaactcgcgcgccaagaccttcgtcccccacccctgcaacatcacgcaggtctacaagtgcgaaggcgaagacgagtgcggccagcccgtcggcgtgtgcgacaagtgggggtgcggcttcaacgagtacaaatggggcgtcgagtccttctacggccggggctcgcagttcgccatcgactcctccaagaagttcaccgtcaccacgcagttcctgaccgacaacggcaaggaggacggcgtcctcgtcgagatccgccgcttgtggcaccaggatggcaagctgatcaagaacaccgctatccaggttgaggagaactacagcacggactcggtgagcaccgagttctgcgagaagactgcttctttcaccatgcagcgcggtggtctcaaggcgatgggcgaggctatcggtcgtggtatggtgctggttttcagcatctgggcggatgattcgggttttatgaactggttggatgcggagggtaatggcccttgcagcgcgactgagggcgatccgaaggagattgtcaagaataagccggatgctagggttacgttctcaaacattaggattggtgaggttggtagcacgtatgctccgggtgggaagtgcggtgttaagagcagggttgctagggggcttactgcttcttaaggggggtgtgaagagaggaggaggtgttgttgggggttggagatgataattgggcgagatggtgtagagcgggttggttggatatgaatacgttgaattggatgt

SEQ ID NO: 30 - protein, 440 aa, Cladorrhinum foecundissimum
MVHKFALLTGLAASLASAQQIGTVVPESHPKLPTKRCTLAGGCQTVDTSIVIDAFQRPLHKIGDPSTPCVVGGPLCPDAKSCAENCALEGVDYASWGIKTEGDALTLNQWMPDPANPGQYKTTTPRTYLVAEDGKNYEDVKLLAKEISFDADVSNLPCGMNGAFYLSEMLMDGGRGDLNPAGAEYGTGYCDAQCFKLDFINGEANIDQKHGACCNEMDIFESNSRAKTFVPHPCNITQVYKCEGEDECGQPVGVCDKWGCGFNEYKWGVESFYGRGSQFAIDSSKKFTVTTQFLTDNGKEDGVLVEIRRLWHQDGKLIKNTAIQVEENYSTDSVSTEFCEKTASFTMQRGGLKAMGEAIGRGMVLVFSIWADDSGFMNWLDAEGNGPCSATEGDPKEIVKNKPDARVTFSNIRIGEVGSTYAPGGKCGVKSRVARGLTAS

SEQ ID NO: 31 - DNA, 1380 bp, Trichoderma reesei
atggcgccctcagttacactgccgttgaccacggccatcctggccattgcccggctcgtcgccgcccagcaaccgggtaccagcacccccgaggtccatcccaagttgacaacctacaagtgtacaaagtccggggggtgcgtggcccaggacacctcggtggtccttgactggaactaccgctggatgcacgacgcaaactacaactcgtgcaccgtcaacggcggcgtcaacaccacgctctgccctgacgaggcgacctgtggcaagaactgcttcatcgagggcgtcgactacgccgcctcgggcgtcacgacctcgggcagcagcctcaccatgaaccagtacatgcccagcagctctggcggctacagcagcgtctctcctcggctgtatctcctggactctgacggtgagtacgtgatgctgaagctcaacggccaggagctgagcttcgacgtcgacctctctgctctgccgtgtggagagaacggctcgctctacctgtctcagatggacgagaacgggggcgccaaccagtataacacggccggtgccaactacgggagcggctactgcgatgctcagtgccccgtccagacatggaggaacggcaccctcaacactagccaccagggcttctgctgcaacgagatggatatcctggagggcaactcgagggcgaatgccttgacccctcactcttgcacggccacggcctgcgactctgccggttgcggcttcaacccctatggcagcggctacaaaagctactacggccccggagataccgttgacacctccaagaccttcaccatcatcacccagttcaacacggacaacggctcgccctcgggcaaccttgtgagcatcacccgcaagtaccagcaaaacggcgtcgacatccccagcgcccagcccggcggcgacaccatctcgtcctgcccgtccgcctcagcctacggcggcctcgccaccatgggcaaggccctgagcagcggcatggtgctcgtgttcagcatttggaacgacaacagccagtacatgaactggctcgacagcggcaacgccggcccctgcagcagcaccgagggcaacccatccaacatcctggccaacaaccccaacacgcacgtcgtcttctccaacatccgctggggagacattgggtctactacgaactcgactgcgcccccgcccccgcctgcgtccagcacgacgttttcgactacacggaggagctcgacgacttcgagcagcccgagctgcacgcagactcactgggggcagtgcggtggcattgggtacagcgggtgcaagacgtgcacgtcgggcactacgtgccagtatagcaacgactactactcgcaatgcctttag

SEQ ID NO: 32 - protein, 459 aa, Trichoderma reesei
MAPSVTLPLTTAILAIARLVAAQQPGTSTPEVHPKLTTYKCTKSGGCVAQDTSVVLDWNYRWMHDANYNSCTVNGGVNTTLCPDEATCGKNCFIEGVDYAASGVTTSGSSLTMNQYMPSSSGGYSSVSPRLYLLDSDGEYVMLKLNGQELSFDVDLSALPCGENGSLYLSQMDENGGANQYNTAGANYGSGYCDAQCPVQTWRNGTLNTSHQGFCCNEMDILEGNSRANALTPHSCTATACDSAGCGFNPYGSGYKSYYGPGDTVDTSKTFTIITQFNTDNGSPSGNLVSITRKYQQNGVDIPSAQPGGDTISSCPSASAYGGLATMGKALSSGMVLVFSIWNDNSQYMNWLDSGNAGPCSSTEGNPSNILANNPNTHVVFSNIRWGDIGSTTNSTAPPPPPASSTTFSTTRRSSTTSSSPSCTQTHWGQCGGIGYSGCKTCTSGTTCQYSNDYYSQCL

SEQ ID NO: 33 - DNA, 1545 bp, Trichoderma reesei
atgtatcggaagttggccgtcatctcggccttcttggccacagctcgtgctcagtcggcctgcactctccaatcggagactcacccgcctctgacatggcagaaatgctcgtctggtggcacgtgcactcaacagacaggctccgtggtcatcgacgccaactggcgctggactcacgctacgaacagcagcacgaactgctacgatggcaacacttggagctcgaccctatgtcctgacaacgagacctgcgcgaagaactgctgtctggacggtgccgcctacgcgtccacgtacggagttaccacgagcggtaacagcctctccattggctttgtcacccagtctgcgcagaagaacgttggcgctcgcctttaccttatggcgagcgacacgacctaccaggaattcaccctgcttggcaacgagttctctttcgatgttgatgtttcgcagctgccgtgcggcttgaacggagctctctacttcgtgtccatggacgcggatggtggcgtgagcaagtatcccaccaacaccgctggcgccaagtacggcacggggtactgtgacagccagtgtccccgcgatctgaagttcatcaatggccaggccaacgttgagggctgggagccgtcatccaacaacgcgaacacgggcattggaggacacggaagctgctgctctgagatggatatctgggaggccaactccatctccgaggctcttaccccccacccttgcacgactgtcggccaggagatctgcgagggtgatgggtgcggcggaacttactccgataacagatatggcggcacttgcgatcccgatggctgcgactggaacccataccgcctgggcaacaccagcttctacggccctggctcaagctttaccctcgataccaccaagaaattgaccgttgtcacccagttcgagacgtcgggtgccatcaaccgatactatgtccagaatggcgtcactttccagcagcccaacgccgagcttggtagttactctggcaacgagctcaacgatgattactgcacagctgaggaggcagaattcggcggatcctctttctcagacaagggcggcctgactcagttcaagaaggctacctctggcggcatggttctggtcatgagtctgtgggatgattactacgccaacatgctgtggctggactccacctacccgacaaacgagacctcctccacacccggtgccgtgcgcggaagctgctccaccagctccggtgtccctgctcaggtcgaatctcagtctcccaacgccaaggtcaccttctccaacatcaagttcggacccattggcagcaccggcaaccctagcggcggcaaccctcccggcggaaacccgcctggcaccaccaccacccgccgcccagccactaccactggaagctctcccggacctacccagtctcactacggccagtgcggcggtattggctacagcggccccacggtctgcgccagcggcacaacttgccaggtcctgaacccttactactctcagtgcctgtaa

SEQ ID NO: 34 - protein, 514 aa, Trichoderma reesei
MYRKLAVISAFLATARAQSACTLQSETHPPLTWQKCSSGGTCTQQTGSVVIDANWRWTHATNSSTNCYDGNTWSSTLCPDNETCAKNCCLDGAAYASTYGVTTSGNSLSIGFVTQSAQKNVGARLYLMASDTTYQEFTLLGNEFSFDVDVSQLPCGLNGALYFVSMDADGGVSKYPTNTAGAKYGTGYCDSQCPRDLKFINGQANVEGWEPSSNNANTGIGGHGSCCSEMDIWEANSISEALTPHPCTTVGQEICEGDGCGGTYSDNRYGGTCDPDGCDWNPYRLGNTSFYGPGSSFTLDTTKKLTVVTQFETSGAINRYYVQNGVTFQQPNAELGSYSGNELNDDYCTAEEAEFGGSSFSDKGGLTQFKKATSGGMVLVMSLWDDYYANMLWLDSTYPTNETSSTPGAVRGSCSTSSGVPAQVESQSPNAKVTFSNIKFGPIGSTGNPSGGNPPGGNPPGTTTTRRPATTTGSSPGPTQSHYGQCGGIGYSGPTVCASGTTCQVLNPYYSQCL

SEQ ID NO: 35 - DNA, 1611 bp, Trichoderma reesei
atgattgtcggcattctcaccacgctggctacgctggccacactcgcagctagtgtgcctctagaggagcggcaagcttgctcaagcgtctggtaattatgtgaaccctctcaagagacccaaatactgagatatgtcaaggggccaatgtggtggccagaattggtcgggtccgacttgctgtgcttccggaagcacatgcgtctactccaacgactattactcccagtgtcttcccggcgctgcaagctcaagctcgtccacgcgcgccgcgtcgacgacttctcgagtatcccccacaacatcccggtcgagctccgcgacgcctccacctggttctactactaccagagtacctccagtcggatcgggaaccgctacgtattcaggcaacccttttgttggggtcactccttgggccaatgcatattacgcctctgaagttagcagcctcgctattcctagcttgactggagccatggccactgctgcagcagctgtcgcaaaggttccctcttttatgtggctgtaggtcctcccggaaccaaggcaatctgttactgaaggctcatcattcactgcagagatactcttgacaagacccctctcatggagcaaaccttggccgacatccgcaccgccaacaagaatggcggtaactatgccggacagtttgtggtgtatgacttgccggatcgcgattgcgctgcccttgcctcgaatggcgaatactctattgccgatggtggcgtcgccaaatataagaactatatcgacaccattcgtcaaattgtcgtggaatattccgatatccggaccctcctggttattggtatgagtttaaacacctgcctccccccccccttcccttcctttcccgccggcatcttgtcgttgtgctaactattgttccctcttccagagcctgactctcttgccaacctggtgaccaacctcggtactccaaagtgtgccaatgctcagtcagcctaccttgagtgcatcaactacgccgtcacacagctgaaccttccaaatgttgcgatgtatttggacgctggccatgcaggatggcttggctggccggcaaaccaagacccggccgctcagctatttgcaaatgtttacaagaatgcatcgtctccgagagctcttcgcggattggcaaccaatgtcgccaactacaacgggtggaacattaccagccccccatcgtacacgcaaggcaacgctgtctacaacgagaagctgtacatccacgctattggacgtcttcttgccaatcacggctggtccaacgccttcttcatcactgatcaaggtcgatcgggaaagcagcctaccggacagcaacagtggggagactggtgcaatgtgatcggcaccggatttggtattcgcccatccgcaaacactggggactcgttgctggattcgtttgtctgggtcaagccaggcggcgagtgtgacggcaccagcgacagcagtgcgccacgatttgactcccactgtgcgctcccagatgccttgcaaccggcgcctcaagctggtgcttggttccaagcctactttgtgcagcttctcacaaacgcaaacccatcgttcctgtaa

SEQ ID NO: 36 - protein, 471 aa, Trichoderma reesei
MIVGILTTLATLATLAASVPLEERQACSSVWGQCGGQNWSGPTCCASGSTCVYSNDYYSQCLPGAASSSSSTRAASTTSRVSPTTSRSSSATPPPGSTTTRVPPVGSGTATYSGNPFVGVTPWANAYYASEVSSLAIPSLTGAMATAAAAVAKVPSFMWLDTLDKTPLMEQTLADIRTANKNGGNYAGQFVVYDLPDRDCAALASNGEYSIADGGVAKYKNYIDTIRQIVVEYSDIRTLLVIEPDSLANLVTNLGTPKCANAQSAYLECINYAVTQLNLPNVAMYLDAGHAGWLGWPANQDPAAQLFANVYKNASSPRALRGLATNVANYNGWNITSPPSYTQGNAVYNEKLYIHAIGRLLANHGWSNAFFITDQGRSGKQPTGQQQWGDWCNVIGTGFGIRPSANTGDSLLDSFVWVKPGGECDGTSDSSAPRFDSHCALPDALQPAPQAGAWFQAYFVQLLTNANPSFL

SEQ ID NO: 37 - DNA, 2046 bp, Humicola insolens
gccgtgaccttgcgcgctttgggtggcggtggcgagtcgtggacggtgcttgctggtcgccggccttcccggcgatccgcgtgatgagagggccaccaacggcgggatgatgctccatggggaacttccccatggagaagagagagaaacttgcggagccgtgatctggggaaagatgctccgtgtctcgtctatataactcgagtctccccgagccctcaacaccaccagctctgatctcaccatccccatcgacaatcacgcaaacacagcagttgtcgggccattccttcagacacatcagtcaccctccttcaaaatgcgtaccgccaagttcgccaccctcgccgcccttgtggcctcggccgccgcccagcaggcgtgcagtctcaccaccgagaggcacccttccctctcttggaacaagtgcaccgccggcggccagtgccagaccgtccaggcttccatcactctcgactccaactggcgctggactcaccaggtgtctggctccaccaactgctacacgggcaacaagtgggatactagcatctgcactgatgccaagtcgtgcgctcagaactgctgcgtcgatggtgccgactacaccagcacctatggcatcaccaccaacggtgattccctgagcctcaagttcgtcaccaagggccagcactcgaccaacgtcggctcgcgtacctacctgatggacggcgaggacaagtatcagagtacgttctatcttcagccttctcgcgccttgaatcctggctaacgtttacacttcacagccttcgagctcctcggcaacgagttcaccttcgatgtcgatgtctccaacatcggctgcggtctcaacggcgccctgtacttcgtctccatggacgccgatggtggtctcagccgctatcctggcaacaaggctggtgccaagtacggtaccggctactgcgatgctcagtgcccccgtgacatcaagttcatcaacggcgaggccaacattgagggctggaccggctccaccaacgaccccaacgccggcgcgggccgctatggtacctgctgctctgagatggatatctgggaagccaacaacatggctactgccttcactcctcacccttgcaccatcattggccagagccgctgcgagggcgactcgtgcggtggcacctacagcaacgagcgctacgccggcgtctgcgaccccgatggctgcgacttcaactcgtaccgccagggcaacaagaccttctacggcaagggcatgaccgtcgacaccaccaagaagatcactgtcgtcacccagttcctcaaggatgccaacggcgatctcggcgagatcaagcgcttctacgtccaggatggcaagatcatccccaactccgagtccaccatccccggcgtcgagggcaattccatcacccaggactggtgcgaccgccagaaggttgcctttggcgacattgacgacttcaaccgcaagggcggcatgaagcagatgggcaaggccctcgccggccccatggtcctggtcatgtccatctgggatgaccacgcctccaacatgctctggctcgactcgaccttccctgtcgatgccgctggcaagcccggcgccgagcgcggtgcctgcccgaccacctcgggtgtccctgctgaggttgaggccgaggcccccaacagcaacgtcgtcttctccaacatccgcttcggccccatcggctcgaccgttgctggtctccccggcgcgggcaacggcggcaacaacggcggcaaccccccgccccccaccaccaccacctcctcggctccggccaccaccaccaccgccagcgctggccccaaggctggccgctggcagcagtgcggcggcatcggcttcactggcccgacccagtgcgaggagccctacatttgcaccaagctcaacgactggtactctcagtgcctgtaaattctgagtcgctgactcgacgatcacggccggtttttgcatgaaaggaaacaaacgaccgcgataaaaatggagggtaatgagatgtc

SEQ ID NO: 38 - protein, 525 aa, Humicola insolens
MRTAKFATLAALVASAAAQQACSLTTERHPSLSWNKCTAGGQCQTVQASITLDSNWRWTHQVSGSTNCYTGNKWDTSICTDAKSCAQNCCVDGADYTSTYGITTNGDSLSLKFVTKGQHSTNVGSRTYLMDGEDKYQTFELLGNEFTFDVDVSNIGCGLNGALYFVSMDADGGLSRYPGNKAGAKYGTGYCDAQCPRDIKFINGEANIEGWTGSTNDPNAGAGRYGTCCSEMDIWEANNMATAFTPHPCTIIGQSRCEGDSCGGTYSNERYAGVCDPDGCDFNSYRQGNKTFYGKGMTVDTTKKITVVTQFLKDANGDLGEIKRFYVQDGKIIPNSESTIPGVEGNSITQDWCDRQKVAFGDIDDFNRKGGMKQMGKALAGPMVLVMSIWDDHASNMLWLDSTFPVDAAGKPGAERGACPTTSGVPAEVEAEAPNSNVVFSNIRFGPIGSTVAGLPGAGNGGNNGGNPPPPTTTTSSAPATTTTASAGPKAGRWQQCGGIGFTGPTQCEEPYICTKLNDWYSQCL

SEQ ID NO: 39 - DNA, 1812 bp, Myceliophthora thermophila
atggccaagaagcttttcatcaccgccgcccttgcggctgccgtgttggcggcccccgtcattgaggagcgccagaactgcggcgctgtgtggtaagaaagcccggtctgagtttcccatgactttctcatcgagtaatggcataaggcccaccccttcgactgactgtgagaatcgatcaaatccaggactcaatgcggcggcaacgggtggcagggtcccacatgctgcgcctcgggctcgacctgcgttgcgcagaacgagtggtactctcagtgcctgcccaacaatcaggtgacgagttccaacactccgtcgtcgacttccacctcgcagcgcagcagcagcacctccagcagcagcaccaggagcggcagctcctcctcctccaccaccacgccccctcccgtctccagccccgtgactagcattcccggcggtgcgaccaccacggcgagctactctggcaaccccttctcgggcgtccggctcttcgccaacgactactacaggtccgaggtccacaatctcgccattcctagcatgaccggtactctggcggccaaggcttccgccgtcgccgaagtccctagcttccagtggctcgaccggaacgtcaccatcgacaccctgatggtccagactctgtcccagatccgggctgccaataatgccggtgccaatcctccctatgctggtgagttacatggcggcgacttgccttctcgtcccccacctttcttgacgggatcggttacctgacctggaggcaaaacaaaaccagcccaacttgtcgtctacgacctccccgaccgtgactgcgccgccgctgcgtccaacggcgagttttcgattgcaaacggcggcgccgccaactacaggagctacatcgacgctatccgcaagcacatcattgagtactcggacatccggatcatcctggttatcgagcccgactcgatggccaacatggtgaccaacatgaacgtggccaagtgcagcaacgccgcgtcgacgtaccacgagttgaccgtgtacgcgctcaagcagctgaacctgcccaacgtcgccatgtatctcgacgccggccacgccggctggctcggctggcccgccaacatccagcccgccgccgacctgtttgccggcatctacaatgacgccggcaagccggctgccgtccgcggcctggccactaacgtcgccaactacaacgcctggagtatcgcttcggccccgtcgtacacgtcccctaaccctaactacgacgagaagcactacatcgaggccttcagcccgctcctgaacgcggccggcttccccgcacgcttcattgtcgacactggccgcaacggcaaacaacctaccggtatggtttttttctttttttttctctgttcccctcccccttccccttcagttggcgtccacaaggtctcttagtcttgcttcttctcggaccaaccttcccccacccccaaaacgcaccgcccacaaccgttcgactctatactcttgggaatgggcgccgaaactgaccgttcgacaggccaacaacagtggggtgactggtgcaatgtcaagggcactggctttggcgtgcgcccgacggccaacacgggccacgacctggtcgatgcctttgtctgggtcaagcccggcggcgagtccgacggcacaagcgacaccagcgccgcccgctacgactaccactgcggcctgtccgatgccctgcagcctgctccggaggctggacagtggttccaggcctacttcgagcagctgctcaccaacgccaacccgcccttctaa

SEQ ID NO: 40 - protein, 482 aa, Myceliophthora thermophila
MAKKLFITAALAAAVLAAPVIEERQNCGAVWTQCGGNGWQGPTCCASGSTCVAQNEWYSQCLPNNQVTSSNTPSSTSTSQRSSSTSSSSTRSGSSSSSTTTPPPVSSPVTSIPGGATTTASYSGNPFSGVRLFANDYYRSEVHNLAIPSMTGTLAAKASAVAEVPSFQWLDRNVTIDTLMVQTLSQIRAANNAGANPPYAAQLVVYDLPDRDCAAAASNGEFSIANGGAANYRSYIDAIRKHIIEYSDIRIILVIEPDSMANMVTNMNVAKCSNAASTYHELTVYALKQLNLPNVAMYLDAGHAGWLGWPANIQPAADLFAGIYNDAGKPAAVRGLATNVANYNAWSIASAPSYTSPNPNYDEKHYIEAFSPLLNAAGFPARFIVDTGRNGKQPTGQQQWGDWCNVKGTGFGVRPTANTGHDLVDAFVWVKPGGESDGTSDTSAARYDYHCGLSDALQPAPEAGQWFQAYFEQLLTNANPPF

SEQ ID NO: 41 - DNA, 1446 bp, Thielavia terrestris
atggctcagaagctccttctcgccgccgcccttgcggccagcgccctcgctgctcccgtcgtcgaggagcgccagaactgcggttccgtctggagccaatgcggcggcattggctggtccggcgcgacctgctgcgcttcgggcaatacctgcgttgagctgaacccgtactactcgcagtgcctgcccaacagccaggtgactacctcgaccagcaagaccacctccaccaccaccaggagcagcaccaccagccacagcagcggtcccaccagcacgagcaccaccaccaccagcagtcccgtggtcactaccccgccgagtacctccatccccggcggtgcctcgtcaacggccagctggtccggcaacccgttctcgggcgtgcagatgtgggccaacgactactacgcctccgaggtctcgtcgctggccatccccagcatgacgggcgccatggccaccaaggcggccgaggtggccaaggtgcccagcttccagtggcttgaccgcaacgtcaccatcgacacgctgttcgcccacacgctgtcgcagatccgcgcggccaaccagaaaggcgccaacccgccctacgcgggcatcttcgtggtctacgaccttccggaccgcgactgcgccgccgccgcgtccaacggcgagttctccatcgcgaacaacggggcggccaactacaagacgtacatcgacgcgatccggagcctcgtcatccagtactcagacatccgcatcatcttcgtcatcgagcccgactcgctggccaacatggtgaccaacctgaacgtggccaagtgcgccaacgccgagtcgacctacaaggagttgaccgtctacgcgctgcagcagctgaacctgcccaacgtggccatgtacctggacgccggccacgccggctggctcggctggcccgccaacatccagccggccgccaacctcttcgccgagatctacacgagcgccggcaagccggccgccgtgcgcggcctcgccaccaacgtggccaactacaacggctggagcctggccacgccgccctcgtacacccagggcgaccccaactacgacgagagccactacgtccaggccctcgccccgctgctcaccgccaacggcttccccgcccacttcatcaccgacaccggccgcaacggcaagcagccgaccggacaacggcaatggggagactggtgcaacgttatcggaactggcttcggcgtgcgcccgacgacaaacaccggcctcgacatcgaggacgccttcgtctgggtcaagcccggcggcgagtgcgacggcacgagcaacacgacctctccccgctacgactaccactgcggcctgtcggacgcgctgcagcctgctccggaggccggcacttggttccaggcctacttcgagcagctcctgaccaacgccaacccgcccttttaa

SEQ ID NO: 42 - protein, 481 aa, Thielavia terrestris
MAQKLLLAAALAASALAAPVVEERQNCGSVWSQCGGIGWSGATCCASGNTCVELNPYYSQCLPNSQVTTSTSKTTSTTTRSSTTSHSSGPTSTSTTTTSSPVVTTPPSTSIPGGASSTASWSGNPFSGVQMWANDYYASEVSSLAIPSMTGAMATKAAEVAKVPSFQWLDRNVTIDTLFAHTLSQIRAANQKGANPPYAGIFVVYDLPDRDCAAAASNGEFSIANNGAANYKTYIDAIRSLVIQYSDIRIIFVIEPDSLANMVTNLNVAKCANAESTYKELTVYALQQLNLPNVAMYLDAGHAGWLGWPANIQPAANLFAEIYTSAGKPAAVRGLATNVANYNGWSLATPPSYTQGDPNYDESHYVQALAPLLTANGFPAHFITDTGRNGKQPTGQRQWGDWCNVIGTGFGVRPTTNTGLDIEDAFVWVKPGGECDGTSNTTSPRYDYHCGLSDALQPAPEAGTWFQAYFEQLLTNANPPF

SEQ ID NO: 43 - DNA, 1593 bp, Chaetomium thermophilum
atgatgtacaagaagttcgccgctctcgccgccctcgtggctggcgccgccgcccagcaggcttgctccctcaccactgagacccaccccagactcacttggaagcgctgcacctctggcggcaactgctcgaccgtgaacggcgccgtcaccatcgatgccaactggcgctggactcacactgtttccggctcgaccaactgctacaccggcaacgagtgggatacctccatctgctctgatggcaagagctgcgcccagacctgctgcgtcgacggcgctgactactcttcgacctatggtatcaccaccagcggtgactccctgaacctcaagttcgtcaccaagcaccagcacggcaccaatgtcggctctcgtgtctacctgatggagaacgacaccaagtaccagatgttcgagctcctcggcaacgagttcaccttcgatgtcgatgtctctaacctgggctgcggtctcaacggcgccctctacttcgtctccatggacgctgatggtggtatgagcaagtactctggcaacaaggctggcgccaagtacggtaccggctactgcgatgctcagtgcccgcgcgaccttaagttcatcaacggcgaggccaacattgagaactggaccccttcgaccaatgatgccaacgccggtttcggccgctatggcagctgctgctctgagatggatatctgggatgccaacaacatggctactgccttcactcctcacccttgcaccattatcggccagagccgctgcgagggcaacagctgcggtggcacctacagctctgagcgctatgctggtgtttgcgatcctgatggctgcgacttcaacgcctaccgccagggcgacaagaccttctacggcaagggcatgaccgtcgacaccaccaagaagatgaccgtcgtcacccagttccacaagaactcggctggcgtcctcagcgagatcaagcgcttctacgttcaggacggcaagatcattgccaacgccgagtccaagatccccggcaaccccggcaactccatcacccaggagtggtgcgatgcccagaaggtcgccttcggtgacatcgatgacttcaaccgcaagggcggtatggctcagatgagcaaggccctcgagggccctatggtcctggtcatgtccgtctgggatgaccactacgccaacatgctctggctcgactcgacctaccccattgacaaggccggcacccccggcgccgagcgcggtgcttgcccgaccacctccggtgtccctgccgagattgaggcccaggtccccaacagcaacgttatcttctccaacatccgcttcggccccatcggctcgaccgtccctggcctcgacggcagcacccccagcaacccgaccgccaccgttgctcctcccacttctaccaccaccagcgtgagaagcagcactactcagatttccaccccgactagccagcccggcggctgcaccacccagaagtggggccagtgcggtggtatcggctacaccggctgcactaactgcgttgctggcactacctgcactgagctcaacccctggtacagccagtgcctgtaa

SEQ ID NO: 44 - protein, 530 aa, Chaetomium thermophilum
MMYKKFAALAALVAGAAAQQACSLTTETHPRLTWKRCTSGGNCSTVNGAVTIDANWRWTHTVSGSTNCYTGNEWDTSICSDGKSCAQTCCVDGADYSSTYGITTSGDSLNLKFVTKHQHGTNVGSRVYLMENDTKYQMFELLGNEFTFDVDVSNLGCGLNGALYFVSMDADGGMSKYSGNKAGAKYGTGYCDAQCPRDLKFINGEANIENWTPSTNDANAGFGRYGSCCSEMDIWDANNMATAFTPHPCTIIGQSRCEGNSCGGTYSSERYAGVCDPDGCDFNAYRQGDKTFYGKGMTVDTTKKMTVVTQFHKNSAGVLSEIKRFYVQDGKIIANAESKIPGNPGNSITQEWCDAQKVAFGDIDDFNRKGGMAQMSKALEGPMVLVMSVWDDHYANMLWLDSTYPIDKAGTPGAERGACPTTSGVPAEIEAQVPNSNVIFSNIRFGPIGSTVPGLDGSTPSNPTATVAPPTSTTTSVRSSTTQISTPTSQPGGCTTQKWGQCGGIGYTGCTNCVAGTTCTELNPWYSQCL

SEQ ID NO: 45 - DNA, 1434 bp, Chaetomium thermophilum
atggctaagcagctgctgctcactgccgctcttgcggccacttcgctggctgcccctctccttgaggagcgccagagctgctcctccgtctggggtcaatgcggtggcatcaattacaacggcccgacctgctgccagtccggcagtgtttgcacttacctgaatgactggtacagccagtgcattcccggtcaggctcagcccggcacgactagcaccacggctcggaccaccagcaccagcaccaccagcacttcgtcggtccgcccgaccacctcgaatacccctgtgacgactgctcccccgacgaccaccatcccgggcggcgcctcgagcacggccagctacaacggcaacccgttttcgggtgttcaactttgggccaacacctactactcgtccgaggtgcacactttggccatccccagcttgtctcctgagctggctgccaaggccgccaaggtcgctgaggttcccagcttccagtggctcgaccgcaatgtgactgttgacactctcttctccggcactcttgccgaaatccgcgccgccaaccagcgcggtgccaacccgccttatgccggcattttcgtggtttatgacttaccagaccgtgattgcgcggctgctgcttcgaacggcgagtggtctatcgccaacaatggtgccaacaactacaagcgctacatcgaccggatccgtgagctccttatccagtactccgatatccgcactattctggtcattgaacctgattccctggccaacatggtcaccaacatgaacgtccagaagtgctcgaacgctgcctccacttacaaggagcttactgtctatgccctcaaacagctcaatcttcctcacgttgccatgtacatggatgctggccacgctggctggcttggctggcccgccaacatccagcctgctgctgagctctttgctcaaatctaccgcgacgctggcaggcccgctgctgtccgcggtcttgcgaccaacgttgccaactacaatgcttggtcgatcgccagccctccgtcctacacctctcctaacccgaactacgacgagaagcactatattgaggcctttgctcctcttctccgcaaccagggcttcgacgcaaagttcatcgtcgacaccggccgtaacggcaagcagcccactggccagcttgaatggggtcactggtgcaatgtcaagggaactggcttcggtgtgcgccctactgctaacactgggcatgaacttgttgatgctttcgtgtgggtcaagcccggtggcgagtccgacggcaccagtgcggacaccagcgctgctcgttatgactatcactgcggcctttccgacgcactgactccggcgcctgaggctggccaatggttccaggcttatttcgaacagctgctcatcaatgccaaccctccgctctga

SEQ ID NO: 46 - protein, 477 aa, Chaetomium thermophilum
MAKQLLLTAALAATSLAAPLLEERQSCSSVWGQCGGINYNGPTCCQSGSVCTYLNDWYSQCIPGQAQPGTTSTTARTTSTSTTSTSSVRPTTSNTPVTTAPPTTTIPGGASSTASYNGNPFSGVQLWANTYYSSEVHTLAIPSLSPELAAKAAKVAEVPSFQWLDRNVTVDTLFSGTLAEIRAANQRGANPPYAGIFVVYDLPDRDCAAAASNGEWSIANNGANNYKRYIDRIRELLIQYSDIRTILVIEPDSLANMVTNMNVQKCSNAASTYKELTVYALKQLNLPHVAMYMDAGHAGWLGWPANIQPAAELFAQIYRDAGRPAAVRGLATNVANYNAWSIASPPSYTSPNPNYDEKHYIEAFAPLLRNQGFDAKFIVDTGRNGKQPTGQLEWGHWCNVKGTGFGVRPTANTGHELVDAFVWVKPGGESDGTSADTSAARYDYHCGLSDALTPAPEAGQWFQAYFEQLLINANPPL

SEQ ID NO: 47 - DNA, 2586 bp, Aspergillus oryzae
atgaagcttggttggatcgaggtggccgcattggcggctgcctcagtagtcagtgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 48 - protein, 861 aa, Aspergillus oryzae
MKLGWIEVAALAAASVVSAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 49 - DNA, 3060 bp, Aspergillus fumigatus
atgagattcggttggctcgaggtggccgctctgacggccgcttctgtagccaatgcccaggtttgtgatgctttcccgtcattgtttcggatatagttgacaatagtcatggaaataatcaggaattggctttctctccaccattctacccttcgccttgggctgatggccagggagagtgggcagatgcccatcgacgcgccgtcgagatcgtttctcagatgacactggcggagaaggttaaccttacaacgggtactgggtgggttgcgacttttttgttgacagtgagctttcttcactgaccatctacacagatgggaaatggaccgatgcgtcggtcaaaccggcagcgttcccaggtaagcttgcaattctgcaacaacgtgcaagtgtagttgctaaaacgcggtggtgcagacttggtatcaactggggtctttgtggccaggattcccctttgggtatccgtttctgtgagctatacccgcggagtctttcagtccttgtattatgtgctgatgattgtctctgtatagctgacctcaactccgccttccctgctggtactaatgtcgccgcgacatgggacaagacactcgcctaccttcgtggcaaggccatgggtgaggaattcaacgacaagggcgtggacattttgctggggcctgctgctggtcctctcggcaaatacccggacggcggcagaatctgggaaggcttctctcctgatccggttctcactggtgtacttttcgccgaaactatcaagggtatccaagacgcgggtgtgattgctactgccaagcattacattctgaatgaacaggagcatttccgacaggttggcgaggcccagggatatggttacaacatcacggagacgatcagctccaacgtggatgacaagaccatgcacgagttgtacctttggtgagtagttgacactgcaaatgaggaccttgattgatttgactgacctggaatgcaggccctttgcagatgctgtgcgcggtaagattttccgtagacttgacctcgcgacgaagaaatcgctgacgaaccatcgtagctggcgttggcgctgtcatgtgttcctacaatcaaatcaacaacagctacggttgtcaaaacagtcaaactctcaacaagctcctcaaggctgagctgggcttccaaggcttcgtcatgagtgactggagcgctcaccacagcggtgtcggcgctgccctcgctgggttggatatgtcgatgcctggagacatttccttcgacgacggactctccttctggggcacgaacctaactgtcagtgttcttaacggcaccgttccagcctggcgtgtcgatgacatggctgttcgtatcatgaccgcgtactacaaggttggtcgtgaccgtcttcgtattccccctaacttcagctcctggacccgggatgagtacggctgggagcattctgctgtctccgagggagcctggaccaaggtgaacgacttcgtcaatgtgcagcgcagtcactctcagatcatccgtgagattggtgccgctagtacagtgctcttgaagaacacgggtgctcttcctttgaccggcaaggaggttaaagtgggtgttctcggtgaagacgctggttccaacccgtggggtgctaacggctgccccgaccgcggctgtgataacggcactcttgctatggcctggggtagtggtactgccaacttcccttaccttgtcacccccgagcaggctatccagcgagaggtcatcagcaacggcggcaatgtctttgctgtgactgataacggggctctcagccagatggcagatgttgcatctcaatccaggtgagtgcgggctcttagaaaaagaacgttctctgaatgaagttttttaaccattgcgaacagcgtgtctttggtgtttgtcaacgccgactctggagagggtttcatcagtgtcgacggcaacgagggtgaccgcaaaaatctcactctgtggaagaacggcgaggccgtcattgacactgttgtcagccactgcaacaacacgattgtggttattcacagtgttgggcccgtcttgatcgaccggtggtatgataaccccaacgtcactgccatcatctgggccggcttgcccggtcaggagagtggcaactccctggtcgacgtgctctatggccgcgtcaaccccagcgccaagaccccgttcacctggggcaagactcgggagtcttacggggctcccttgctcaccgagcctaacaatggcaatggtgctccccaggatgatttcaacgagggcgtcttcattgactaccgtcactttgacaagcgcaatgagacccccatttatgagtttggccatggcttgagctacaccacctttggttactctcaccttcgggttcaggccctcaatagttcgagttcggcatatgtcccgactagcggagagaccaagcctgcgccaacctatggtgagatcggtagtgccgccgactacctgtatcccgagggtctcaaaagaattaccaagtttatttacccttggctcaactcgaccgacctcgaggattcttctgacgacccgaactacggctgggaggactcggagtacattcccgaaggcgctagggatgggtctcctcaacccctcctgaaggctggcggcgctcctggtggtaaccctaccctttatcaggatcttgttagggtgtcggccaccataaccaacactggtaacgtcgccggttatgaagtccctcaattggtgagtgacccgcatgttccttgcgttgcaatttggctaactcgcttctagtatgtttcactgggcggaccgaacgagcctcgggtcgttctgcgcaagttcgaccgaatcttcctggctcctggggagcaaaaggtttggaccacgactcttaaccgtcgtgatctcgccaattgggatgtggaggctcaggactgggtcatcacaaagtaccccaagaaagtgcacgtcggcagctcctcgcgtaagctgcctctgagagcgcctctgccccgtgtctactag

SEQ ID NO: 50 - protein, 863 aa, Aspergillus fumigatus
MRFGWLEVAALTAASVANAQELAFSPPFYPSPWADGQGEWADAHRRAVEIVSQMTLAEKVNLTTGTGWEMDRCVGQTGSVPRLGINWGLCGQDSPLGIRFSDLNSAFPAGTNVAATWDKTLAYLRGKAMGEEFNDKGVDILLGPAAGPLGKYPDGGRIWEGFSPDPVLTGVLFAETIKGIQDAGVIATAKHYILNEQEHFRQVGEAQGYGYNITETISSNVDDKTMHELYLWPFADAVRAGVGAVMCSYNQINNSYGCQNSQTLNKLLKAELGFQGFVMSDWSAHHSGVGAALAGLDMSMPGDISFDDGLSFWGTNLTVSVLNGTVPAWRVDDMAVRIMTAYYKVGRDRLRIPPNFSSWTRDEYGWEHSAVSEGAWTKVNDFVNVQRSHSQIIREIGAASTVLLKNTGALPLTGKEVKVGVLGEDAGSNPWGANGCPDRGCDNGTLAMAWGSGTANFPYLVTPEQAIQREVISNGGNVFAVTDNGALSQMADVASQSSVSLVFVNADSGEGFISVDGNEGDRKNLTLWKNGEAVIDTVVSHCNNTIVVIHSVGPVLIDRWYDNPNVTAIIWAGLPGQESGNSLVDVLYGRVNPSAKTPFTWGKTRESYGAPLLTEPNNGNGAPQDDFNEGVFIDYRHFDKRNETPIYEFGHGLSYTTFGYSHLRVQALNSSSSAYVPTSGETKPAPTYGEIGSAADYLYPEGLKRITKFIYPWLNSTDLEDSSDDPNYGWEDSEYIPEGARDGSPQPLLKAGGAPGGNPTLYQDLVRVSATITNTGNVAGYEVPQLYVSLGGPNEPRVVLRKFDRIFLAPGEQKVWTTTLNRRDLANWDVEAQDWVITKYPKKVHVGSSSRKLPLRAPLPRVY

SEQ ID NO: 51 - DNA, 2800 bp, Penicillium brasilianum
tgaaaatgcagggttctacaatctttctggctttcgcctcatgggcgagccaggttgctgccattgcgcagcccatacagaagcacgaggtttgttttatcttgctcatggacgtgctttgacttgactaattgttttacatacagcccggatttctgcacgggccccaagccatagaatcgttctcagaaccgttctacccgtcgccctggatgaatcctcacgccgagggctgggaggccgcatatcagaaagctcaagattttgtctcgcaactcactatcttggagaaaataaatctgaccaccggtgttgggtaagtctctccgactgcttctgggtcacggtgcgacgagccactgactttttgaagctgggaaaatgggccgtgtgtaggaaacactggatcaattcctcgtctcggattcaaaggattttgtacccaggattcaccacagggtgttcggttcgcagattattcctccgctttcacatctagccaaatggccgccgcaacatttgaccgctcaattctttatcaacgaggccaagccatggcacaggaacacaaggctaagggtatcacaattcaattgggccctgttgccggccctctcggtcgcatccccgagggcggccgcaactgggaaggattctcccctgatcctgtcttgactggtatagccatggctgagacaattaagggcatgcaggatactggagtgattgcttgcgctaaacattatattggaaacgagcaggagcacttccgtcaagtgggtgaagctgcgggtcacggatacactatttccgatactatttcatctaatattgacgaccgtgctatgcatgagctatacttgtggccatttgctgatgccgttcgcgctggtgtgggttctttcatgtgctcatactctcagatcaacaactcctacggatgccaaaacagtcagaccctcaacaagctcctcaagagcgaattgggcttccaaggctttgtcatgagcgattggggtgcccatcactctggagtgtcatcggcgctagctggacttgatatgagcatgccgggtgataccgaatttgattctggcttgagcttctggggctctaacctcaccattgcaattctgaacggcacggttcccgaatggcgcctggatgacatggcgatgcgaattatggctgcatacttcaaagttggccttactattgaggatcaaccagatgtcaacttcaatgcctggacccatgacacctacggatataaatacgcttatagcaaggaagattacgagcaggtcaactggcatgtcgatgttcgcagcgaccacaataagctcattcgcgagactgccgcgaagggtacagttctgctgaagaacaactttcatgctctccctctgaagcagcccaggttcgtggccgtcgttggtcaggatgccgggccaaaccccaagggccctaacggctgcgcagaccgaggatgcgaccaaggcactctcgcaatgggatggggctcagggtctaccgaattcccttacctggtcactcctgacactgctattcagtcaaaggtcctcgaatacgggggtcgatacgagagtatttttgataactatgacgacaatgctatcttgtcgcttgtctcacagcctgatgcaacctgtatcgtttttgcaaatgccgattccggtgaaggctacatcactgtcgacaacaactggggtgaccgcaacaatctgaccctctggcaaaatgccgatcaagtgattagcactgtcagctcgcgatgcaacaacacaatcgttgttctccactctgtcggaccagtgttgctaaatggtatatatgagcacccgaacatcacagctattgtctgggcagggatgccaggcgaagaatctggcaatgctctcgtggatattctttggggcaatgttaaccctgccggtcgcactccgttcacctgggccaaaagtcgagaggactatggcactgatataatgtacgagcccaacaacggccagcgtgcgcctcagcaggatttcaccgagagcatctacctcgactaccgccatttcgacaaagctggtatcgagccaatttacgagtttggattcggcctctcctataccaccttcgaatactctgacctccgtgttgtgaagaagtatgttcaaccatacagtcccacgaccggcaccggtgctcaagcaccttccatcggacagccacctagccagaacctggatacctacaagttccctgctacatacaagtacatcaaaaccttcatttatccctacctgaacagcactgtctccctccgcgctgcttccaaggatcccgaatacggtcgtacagactttatcccaccccacgcgcgtgatggctcccctcaacctctcaaccccgctggagacccagtggccagtggtggaaacaacatgctctacgacgaactttacgaggtcactgcacagatcaaaaacactggcgacgtggccggcgacgaagtcgtccagctttacgtagatctcgggggtgacaacccgcctcgtcagttgagaaactttgacaggttttatctgctgcccggtcagagctcaacattccgggctacattgacgcgccgtgatttgagcaactgggatattgaggcgcagaactggcgagttacggaatcgcctaagagagtgtatgttggacggtcgagtcgggatttgccgctgagctcacaattggagtaatgatcatgtctaccaatagatgttgaatgtctggtgtggatatt

SEQ ID NO: 52 - protein, 878 aa, Penicillium brasilianum
MQGSTIFLAFASWASQVAAIAQPIQKHEPGFLHGPQAIESFSEPFYPSPWMNPHAEGWEAAYQKAQDFVSQLTILEKINLTTGVGWENGPCVGNTGSIPRLGFKGFCTQDSPQGVRFADYSSAFTSSQMAAATFDRSILYQRGQAMAQEHKAKGITIQLGPVAGPLGRIPEGGRNWEGFSPDPVLTGIAMAETIKGMQDTGVIACAKHYIGNEQEHFRQVGEAAGHGYTISDTISSNIDDRAMHELYLWPFADAVRAGVGSFMCSYSQINNSYGCQNSQTLNKLLKSELGFQGFVMSDWGAHHSGVSSALAGLDMSMPGDTEFDSGLSFWGSNLTIAILNGTVPEWRLDDMAMRIMAAYFKVGLTIEDQPDVNFNAWTHDTYGYKYAYSKEDYEQVNWHVDVRSDHNKLIRETAAKGTVLLKNNFHALPLKQPRFVAVVGQDAGPNPKGPNGCADRGCDQGTLAMGWGSGSTEFPYLVTPDTAIQSKVLEYGGRYESIFDNYDDNAILSLVSQPDATCIVFANADSGEGYITVDNNWGDRNNLTLWQNADQVISTVSSRCNNTIVVLHSVGPVLLNGIYEHPNITAIVWAGMPGEESGNALVDILWGNVNPAGRTPFTWAKSREDYGTDIMYEPNNGQRAPQQDFTESIYLDYRHFDKAGIEPIYEFGFGLSYTTFEYSDLRVVKKYVQPYSPTTGTGAQAPSIGQPPSQNLDTYKFPATYKYIKTFIYPYLNSTVSLRAASKDPEYGRTDFIPPHARDGSPQPLNPAGDPVASGGNNMLYDELYEVTAQIKNTGDVAGDEVVQLYVDLGGDNPPRQLRNFDRFYLLPGQSSTFRATLTRRDLSNWDIEAQNWRVTESPKRVYVGRSSRDLPLSSQLE

SEQ ID NO: 53 - DNA, 2583 bp, Aspergillus niger
atgaggttcactttgatcgaggcggtggctctgactgccgtctcgctggccagcgctgatgaattggcctactccccaccgtattacccatccccttgggccaatggccagggcgactgggcgcaggcataccagcgcgctgttgatattgtctcgcaaatgacattggatgagaaggtcaatctgaccacaggaactggatgggaattggaactatgtgttggtcagactggcggtgttccccgattgggagttccgggaatgtgtttacaggatagccctctgggcgttcgcgactccgactacaactctgctttccctgccggcatgaacgtggctgcaacctgggacaagaatctggcataccttcgcggcaaggctatgggtcaggaatttagtgacaagggtgccgatatccaattgggtccagctgccggccctctcggtagaagtcccgacggtggtcgtaactgggagggcttctccccagaccctgccctaagtggtgtgctctttgccgagaccatcaagggtatccaagatgctggtgtggttgcgacggctaagcactacattgcttacgagcaagagcatttccgtcaggcgcctgaagcccaaggttttggatttaatatttccgagagtggaagtgcgaacctcgatgataagactatgcacgagctgtacctctggcccttcgcggatgccatccgtgcaggtgctggcgctgtgatgtgctcctacaaccagatcaacaacagttatggctgccagaacagctacactctgaacaagctgctcaaggccgagctgggcttccagggctttgtcatgagtgattgggctgctcaccatgctggtgtgagtggtgctttggcaggattggatatgtctatgccaggagacgtcgactacgacagtggtacgtcttactggggtacaaacttgaccattagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactacgtgtcggagggaccgtacgagaaggtcaaccagtacgtgaatgtgcaacgcaaccacagcgaactgattcgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgcttaagcacaagaatggtgtattcaccgccaccgataactgggctatcgatcagattgaggcgcttgctaagaccgccagtgtctctcttgtctttgtcaacgccgactctggtgagggttacatcaatgtggacggaaacctgggtgaccgcaggaacctgaccctgtggaggaacggcgataatgtgatcaaggctgctgctagcaactgcaacaacacaatcgttgtcattcactctgtcggaccagtcttggttaacgagtggtacgacaaccccaatgttaccgctatcctctggggtggtttgcccggtcaggagtctggcaactctcttgccgacgtcctctatggccgtgtcaaccccggtgccaagtcgccctttacctggggcaagactcgtgaggcctaccaagactacttggtcaccgagcccaacaacggcaacggagcccctcaggaagactttgtcgagggcgtcttcattgactaccgtggatttgacaagcgcaacgagaccccgatctacgagttcggctatggtctgagctacaccactttcaactactcgaaccttgaggtgcaggtgctgagcgcccctgcatacgagcctgcttcgggtgagaccgaggcagcgccaaccttcggagaggttggaaatgcgtcggattacctctaccccagcggattgcagagaattaccaagttcatctacccctggctcaacggtaccgatctcgaggcatcttccggggatgctagctacgggcaggactcctccgactatcttcccgagggagccaccgatggctctgcgcaaccgatcctgcctgccggtggcggtcctggcggcaaccctcgcctgtacgacgagctcatccgcgtgtcagtgaccatcaagaacaccggcaaggttgctggtgatgaagttccccaactgtatgtttcccttggcggtcccaatgagcccaagatcgtgctgcgtcaattcgagcgcatcacgctgcagccgtcggaggagacgaagtggagcacgactctgacgcgccgtgaccttgcaaactggaatgttgagaagcaggactgggagattacgtcgtatcccaagatggtgtttgtcggaagctcctcgcggaagctgccgctccgggcgtctctgcctactgttcactaa

SEQ ID NO: 54 - protein, 860 aa, Aspergillus niger
MRFTLIEAVALTAVSLASADELAYSPPYYPSPWANGQGDWAQAYQRAVDIVSQMTLDEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGFGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKHKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASDYLYPSGLQRITKFIYPWLNGTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 55 - DNA, 2583 bp, Aspergillus aculeatus
atgaagctcagttggcttgaggcggctgccttgacggctgcttcagtcgtcagcgctgatgaactggcgttctctcctcctttctacccctctccgtgggccaatggccagggagagtgggcggaagcctaccagcgtgcagtggccattgtatcccagatgactctggatgagaaggtcaacctgaccaccggaactggatgggagctggagaagtgcgtcggtcagactggtggtgtcccaagactgaacatcggtggcatgtgtcttcaggacagtcccttgggaattcgtgatagtgactacaattcggctttccctgctggtgtcaacgttgctgcgacatgggacaagaaccttgcttatctacgtggtcaggctatgggtcaagagttcagtgacaaaggaattgatgttcaattgggaccggccgcgggtcccctcggcaggagccctgatggaggtcgcaactgggaaggtttctctccagacccggctcttactggtgtgctctttgcggagacgattaagggtattcaagacgctggtgtcgtggcgacagccaagcattacattctcaatgagcaagagcatttccgccaggtcgcagaggctgcgggctacggattcaatatctccgacacgatcagctctaacgttgatgacaagaccattcatgaaatgtacctctggcccttcgcggatgccgttcgcgccggcgttggcgccatcatgtgttcctacaaccagatcaacaacagctacggttgccagaacagttacactctgaacaagcttctgaaggccgagctcggcttccagggctttgtgatgtctgactggggtgctcaccacagtggtgttggctctgctttggccggcttggatatgtcaatgcctggcgatatcaccttcgattctgccactagtttctggggtaccaacctgaccattgctgtgctcaacggtaccgtcccgcagtggcgcgttgacgacatggctgtccgtatcatggctgcctactacaaggttggccgcgaccgcctgtaccagccgcctaacttcagctcctggactcgcgatgaatacggcttcaagtatttctacccccaggaagggccctatgagaaggtcaatcactttgtcaatgtgcagcgcaaccacagcgaggttattcgcaagttgggagcagacagtactgttctactgaagaacaacaatgccctgccgctgaccggaaaggagcgcaaagttgcgatcctgggtgaagatgctggatccaactcgtacggtgccaatggctgctctgaccgtggctgtgacaacggtactcttgctatggcttggggtagcggcactgccgaattcccatatctcgtgacccctgagcaggctattcaagccgaggtgctcaagcataagggcagcgtctacgccatcacggacaactgggcgctgagccaggtggagaccctcgctaaacaagccagtgtctctcttgtatttgtcaactcggacgcgggagagggctatatctccgtggacggaaacgagggcgaccgcaacaacctcaccctctggaagaacggcgacaacctcatcaaggctgctgcaaacaactgcaacaacaccatcgttgtcatccactccgttggacctgttttggttgacgagtggtatgaccaccccaacgttactgccatcctctgggcgggcttgcctggccaggagtctggcaactccttggctgacgtgctctacggccgcgtcaacccgggcgccaaatctccattcacctggggcaagacgagggaggcgtacggggattaccttgtccgtgagctcaacaacggcaacggagctccccaagatgatttctcggaaggtgttttcattgactaccgcggattcgacaagcgcaatgagaccccgatctacgagttcggacatggtctgagctacaccactttcaactactctggccttcacatccaggttctcaacgcttcctccaacgctcaagtagccactgagactggcgccgctcccaccttcggacaagtcggcaatgcctctgactacgtgtaccctgagggattgaccagaatcagcaagttcatctatccctggcttaattccacagacctgaaggcctcatctggcgacccgtactatggagtcgacaccgcggagcacgtgcccgagggtgctactgatggctctccgcagcccgttctgcctgccggtggtggctctggtggtaacccgcgcctctacgatgagttgatccgtgtttcggtgacagtcaagaacactggtcgtgttgccggtgatgctgtgcctcaattgtatgtttcccttggtggacccaatgagcccaaggttgtgttgcgcaaattcgaccgcctcaccctcaagccctccgaggagacggtgtggacgactaccctgacccgccgcgatctgtctaactgggacgttgcggctcaggactgggtcatcacttcttacccgaagaaggtccatgttggtagctcttcgcgtcagctgccccttcacgcggcgctcccgaaggtgcaatga

SEQ ID NO: 56 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 57 - DNA, 3294 bp, Aspergillus oryzae
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggtggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatccccatgcgttcctcccccctcctccgctccgccgttgtggccgccctgccggtgttggcccttgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaggtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgtcatgtgctcttacaaccaaatcaacaacagctacggttgcgagaatagcgaaactctgaacaagcttttgaaggcggagcttggtttccaaggcttcgtcatgagtgattggaccgctcatcacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 58 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAHHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 59 - DNA, 3294 bp, Aspergillus oryzae
atgcgttcctcccccctcctccgctccgccgttgtggccgccctgccggtgttggcccttgccgctgatggcaggtccacccgctactgggactgctgcaagccttcgtgcggctgggccaagaaggctcccgtgaaccagcctgtcttttcctgcaacgccaacttccagcgtatcacggacttcgacgccaagtccggctgcgagccgggcggtgtcgcctactcgtgcgccgaccagaccccatgggctgtgaacgacgacttcgcgctcggttttgctgccacctctattgccggcagcaatgaggcgggctggtgctgcgcctgctacgagctcaccttcacatccggtcctgttgctggcaagaagatggtcgtccagtccaccagcactggcggtgatcttggcagcaaccacttcgatctcaacatccccggcggcggcgtcggcatcttcgacggatgcactccccagttcggtggtctgcccggccagcgctacggcggcatctcgtcccgcaacgagtgcgatcggttccccgacgccctcaagcccggctgctactggcgcttcgactggttcaagaacgccgacaatccgagcttcagcttccgtcaggtccagtgcccagccgagctcgtcgctcgcaccggatgccgccgcaacgacgacggcaacttccctgccgtccagatccccatgcgttcctcccccctcctccgctccgccgttgtggccgccctgccggtgttggcccttgccaaggatgatctcgcgtactcccctcctttctacccttccccatgggcagatggtcagggtgaatgggcggaagtatacaaacgcgctgtagacatagtttcccagatgacgttgacagagaaagtcaacttaacgactggaacaggatggcaactagagaggtgtgttggacaaactggcagtgttcccagactcaacatccccagcttgtgtttgcaggatagtcctcttggtattcgtttctcggactacaattcagctttccctgcgggtgttaatgtcgctgccacctgggacaagacgctcgcctaccttcgtggtcaggcaatgggtgaggagttcagtgataagggtattgacgttcagctgggtcctgctgctggccctctcggtgctcatccggatggcggtagaaactgggaaagtttctcaccagatccagccctcaccggtgtactttttgcggagacgattaagggtattcaagatgctggtgtcattgcgacagctaagcattatatcatgaacgaacaagagcatttccgccaacaacccgaggctgcgggttacggattcaacgtaagcgacagtttgagttccaacgttgatgacaagactatgcatgaattgtacctctggcccttcgcggatgcagtacgcgctggagtcggtgctgttatgtgctcttacaaccaaatcaacaacagctacggttgcgagaatagcgaaactctgaacaagcttttgaaggcggagcttggtttccaaggcttcgtcatgagtgattggaccgctcaacacagcggcgtaggcgctgctttagcaggtctggatatgtcgatgcccggtgatgttaccttcgatagtggtacgtctttctggggtgcaaacttgacggtcggtgtccttaacggtacaatcccccaatggcgtgttgatgacatggctgtccgtatcatggccgcttattacaaggttggccgcgacaccaaatacacccctcccaacttcagctcgtggaccagggacgaatatggtttcgcgcataaccatgtttcggaaggtgcttacgagagggtcaacgaattcgtggacgtgcaacgcgatcatgccgacctaatccgtcgcatcggcgcgcagagcactgttctgctgaagaacaagggtgccttgcccttgagccgcaaggaaaagctggtcgcccttctgggagaggatgcgggttccaactcgtggggcgctaacggctgtgatgaccgtggttgcgataacggtacccttgccatggcctggggtagcggtactgcgaatttcccatacctcgtgacaccagagcaggcgattcagaacgaagttcttcagggccgtggtaatgtcttcgccgtgaccgacagttgggcgctcgacaagatcgctgcggctgcccgccaggccagcgtatctctcgtgttcgtcaactccgactcaggagaaggctatcttagtgtggatggaaatgagggcgatcgtaacaacatcactctgtggaagaacggcgacaatgtggtcaagaccgcagcgaataactgtaacaacaccgttgtcatcatccactccgtcggaccagttttgatcgatgaatggtatgaccaccccaatgtcactggtattctctgggctggtctgccaggccaggagtctggtaactccattgccgatgtgctgtacggtcgtgtcaaccctggcgccaagtctcctttcacttggggcaagacccgggagtcgtatggttctcccttggtcaaggatgccaacaatggcaacggagcgccccagtctgatttcacccagggtgttttcatcgattaccgccatttcgataagttcaatgagacccctatctacgagtttggctacggcttgagctacaccaccttcgagctctccgacctccatgttcagcccctgaacgcgtcccgatacactcccaccagtggcatgactgaagctgcaaagaactttggtgaaattggcgatgcgtcggagtacgtgtatccggaggggctggaaaggatccatgagtttatctatccctggatcaactctaccgacctgaaggcatcgtctgacgattctaactacggctgggaagactccaagtatattcccgaaggcgccacggatgggtctgcccagccccgtttgcccgctagtggtggtgccggaggaaaccccggtctgtacgaggatcttttccgcgtctctgtgaaggtcaagaacacgggcaatgtcgccggtgatgaagttcctcagctgtacgtttccctaggcggcccgaatgagcccaaggtggtactgcgcaagtttgagcgtattcacttggccccttcgcaggaggccgtgtggacaacgacccttacccgtcgtgaccttgcaaactgggacgtttcggctcaggactggaccgtcactccttaccccaagacgatctacgttggaaactcctcacggaaactgccgctccaggcctcgctgcctaaggcccagtaa

SEQ ID NO: 60 - protein, 1097 aa, Aspergillus oryzae
MRSSPLLRSAVVAALPVLALAADGRSTRYWDCCKPSCGWAKKAPVNQPVFSCNANFQRITDFDAKSGCEPGGVAYSCADQTPWAVNDDFALGFAATSIAGSNEAGWCCACYELTFTSGPVAGKKMVVQSTSTGGDLGSNHFDLNIPGGGVGIFDGCTPQFGGLPGQRYGGISSRNECDRFPDALKPGCYWRFDWFKNADNPSFSFRQVQCPAELVARTGCRRNDDGNFPAVQIPMRSSPLLRSAVVAALPVLALAKDDLAYSPPFYPSPWADGQGEWAEVYKRAVDIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWESFSPDPALTGVLFAETIKGIQDAGVIATAKHYIMNEQEHFRQQPEAAGYGFNVSDSLSSNVDDKTMHELYLWPFADAVRAGVGAVMCSYNQINNSYGCENSETLNKLLKAELGFQGFVMSDWTAQHSGVGAALAGLDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLIDEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASRYTPTSGMTEAAKNFGEIGDASEYVYPEGLERIHEFIYPWINSTDLKASSDDSNYGWEDSKYIPEGATDGSAQPRLPASGGAGGNPGLYEDLFRVSVKVKNTGNVAGDEVPQLYVSLGGPNEPKVVLRKFERIHLAPSQEAVWTTTLTRRDLANWDVSAQDWTVTPYPKTIYVGNSSRKLPLQASLPKAQ

SEQ ID NO: 61 - DNA, 1846 bp, Thielavia terrestris
aattgaaggagggagtggcggagtggccaccaagtcaggcggctgtcaactaaccaaggatgggaacagttcggctcgccttgcccgagggcagcgttccctgatggggacgaaccatgggactggggtcagctgctgtataaaagttcaaatcgatgatctctcagatggcgctgctggggtgttctgcgcttttccatcctcgcaacctggtatcccactagtccagcgttcggcaccatgaagtcgttcaccattgccgccttggcagccctatgggcccaggaggccgccgcccacgcgaccttccaggacctctggattgatggagtcgactacggctcgcaatgtgtccgcctcccggcgtccaactcccccgtcaccaatgttgcgtccgacgatatccgatgcaatgtcggcacctcgaggcccaccgtcaagtgcccggtcaaggccggctccacggtcacgatcgagatgcaccaggttcgcacgcctctctgcgtaggccccccagctactatatggcactaacacgacctccagcaacctggcgaccggtcttgcgccaacgaggctatcggcggcgaccactacggccccgtaatggtgtacatgtccaaggtcgatgacgcggtgacagccgacggttcatcgggctggttcaaggtgttccaggacagctgggccaagaacccgtcgggttcgacgggcgacgacgactactggggcaccaaggacctcaactcgtgctgcggcaagatgaacgtcaagatccccgaagacatcgagccgggcgactacctgctccgcgccgaggttatcgcgctgcacgtggccgccagctcgggcggcgcgcagttctacatgtcctgctaccagctgaccgtgacgggctccggcagcgccaccccctcgaccgtgaatttcccgggcgcctactcggccagcgacccgggcatcctgatcaacatccacgcgcccatgtcgacctacgtcgtcccgggcccgaccgtgtacgcgggcggctcgaccaagtcggctggcagctcctgctccggctgcgaggcgacctgcacggttggttccggccccagcgcgacactgacgcagcccacctccaccgcgaccgcgacctccgcccctggcggcggcggctccggctgcacggcggccaagtaccagcagtgcggcggcaccggctacactgggtgcaccacctgcgctgtaagttccctcgtgatatgcagcggaacaccgtctggactgttttgctaactcgcgtcgtagtccgggtctacctgcagcgccgtctcgcctccgtactactcgcagtgcctctaagccgggagcgcttgctcagcgggctgctgtgaaggagctccatgtccccatgccgccatggccggagtaccgggctgagcgcccaattcttgtatatagttgagttttcccaatcatgaatacatatgcatctgcatggactgttgcgtcgtcagtctacatcctttgctccactgaactgtgagaccccatgtcatccggaccattcgatcggtgctcgctctaccatctcggttgatgggtctgggcttgagagtcactggcacgtcctcggcggtaatgaaatgtggaggaaagtgtgagctgtctgacgcactcggcgctgatgagacgttgagcgcggcccacactggtgttctgtaagccagcacacaaaagaatactccaggatggcccatagcggcaaatatacagtatcagggatgcaaaaagtgcaaaagtaaggggctcaatcggggatcgaacccgagacctcgcacatgacttatttcaagtcaggggt

SEQ ID NO: 62 - protein, 326 aa, Thielavia terrestris
MKSFTIAALAALWAQEAAAHATFQDLWIDGVDYGSQCVRLPASNSPVTNVASDDIRCNVGTSRPTVKCPVKAGSTVTIEMHQQPGDRSCANEAIGGDHYGPVMVYMSKVDDAVTADGSSGWFKVFQDSWAKNPSGSTGDDDYWGTKDLNSCCGKMNVKIPEDIEPGDYLLRAEVIALHVAASSGGAQFYMSCYQLTVTGSGSATPSTVNFPGAYSASDPGILINIHAPMSTYVVPGPTVYAGGSTKSAGSSCSGCEATCTVGSGPSATLTQPTSTATATSAPGGGGSGCTAAKYQQCGGTGYTGCTTCASGSTCSAVSPPYYSQCL

SEQ ID NO: 63 - DNA, 880 bp, Thielavia terrestris
accccgggatcactgcccctaggaaccagcacacctcggtccaatcatgcggttcgacgccctctccgccctcgctcttgcgccgcttgtggctggccacggcgccgtgaccagctacatcatcggcggcaaaacctatcccggctacgagggcttctcgcctgcctcgagcccgccgacgatccagtaccagtggcccgactacaacccgaccctgagcgtgaccgacccgaagatgcgctgcaacggcggcacctcggcagagctcagcgcgcccgtccaggccggcgagaacgtgacggccgtctggaagcagtggacccaccagcaaggccccgtcatggtctggatgttcaagtgccccggcgacttctcgtcgtgccacggcgacggcaagggctggttcaagatcgaccagctgggcctgtggggcaacaacctcaactcgaacaactggggcaccgcgatcgtctacaagaccctccagtggagcaacccgatccccaagaacctcgcgccgggcaactacctcatccgccacgagctgctcgccctgcaccaggccaacacgccgcagttctacgccgagtgcgcccagctggtcgtctccggcagcggctccgccctgcccccgtccgactacctctacagcatccccgtctacgcgccccagaacgaccccggcatcaccgtgagtgggcttccgttccgcggcgagctctgtggaaatcttgctgacgatgggctaggttgacatctacaacggcgggcttacctcctacaccccgcccggcggccccgtctggtctggcttcgagttttaggcgcattgagtcgggggctacgaggggaaggcatctgttcgcatgagcgtgggtac

SEQ ID NO: 64 - protein, 478 aa, Thielavia terrestris
MRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEFMRFDALSALALAPLVAGHGAVTSYIIGGKTYPGYEGFSPASSPPTIQYQWPDYNPTLSVTDPKMRCNGGTSAELSAPVQAGENVTAVWKQWTHQQGPVMVWMFKCPGDFSSSHGDGKGWFKIDQLGLWGNNLNSNNWGTAIVYKTLQWSNPIPKNLAPGNYLIRHELLALHQANTPQFYAECAQLVVSGSGSALPPSDYLYSIPVYAPQNDPGITVDIYNGGLTSYTPPGGPVWSGFEF

SEQ ID NO: 65 - DNA, 1000 bp, Thielavia terrestris
ctcctgttcctgggccaccgcttgttgcctgcactattggtagagttggtctattgctagagttggccatgcttctcacatcagtcctcggctcggctgccctgcttgctagcggcgctgcggcacacggcgccgtgaccagctacatcatcgccggcaagaattacccggggtgggtagctgattattgagggcgcattcaaggttcataccggtgtgcatggctgacaaccggctggcagataccaaggcttttctcctgcgaactcgccgaacgtcatccaatggcaatggcatgactacaaccccgtcttgtcgtgcagcgactcgaagcttcgctgcaacggcggcacgtcggccaccctgaacgccacggccgcaccgggcgacaccatcaccgccatctgggcgcagtggacgcacagccagggccccatcctggtgtggatgtacaagtgcccgggctccttcagctcctgtgacggctccggcgctggctggttcaagatcgacgaggccggcttccacggcgacggcgtcaaggtcttcctcgacaccgagaacccgtccggctgggacatcgccaagctcgtcggcggcaacaagcagtggagcagcaaggtccccgagggcctcgcccccggcaactacctcgtccgccacgagttgatcgccctgcaccaggccaacaacccgcagttctacccggagtgcgcccaggtcgtcatcaccggctccggcaccgcgcagccggatgcctcatacaaggcggctatccccggctactgcaaccagaatgacccgaacatcaaggtgagatccaggcgtaatgcagtctactgctggaaagaaagtggtccaagctaaaccgcgctccaggtgcccatcaacgaccactccatccctcagacctacaagattcccggccctcccgtcttcaagggcaccgccagcaagaaggcccgggacttcaccgcctgaagttgttgaatcgatggag

SEQ ID NO: 66 - protein, 516 aa, Thielavia terrestris
MLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTAMLLTSVLGSAALLASGAAAHGAVTSYIIAGKNYPGYQGFSPANSPNVIQWQWHDYNPVLSCSDSKLRCNGGTSATLNATAAPGDTITAIWAQWTHSQGPILVWMYKCPGSFSSCDGSGAGWFKIDEAGFHGDGVKVFLDTENPSGWDIAKLVGGNKQWSSKVPEGLAPGNYLVRHELIALHQANNPQFYPECAQVVITGSGTAQPDASYKAAIPGYCNQNDPNIKVPINDHSIPQTYKIPGPPVFKGTASKKARDFTA

SEQ ID NO: 67 - DNA, 681 bp, Thielavia terrestris
atgctcgcaaacggtgccatcgtcttcctggccgccgccctcggcgtcagtggccactacacctggccacgggttaacgacggcgccgactggcaacaggtccgtaaggcggacaactggcaggacaacggctacgtcggggatgtcacgtcgccacagatccgctgtttccaggcgaccccgtccccggccccatccgtcctcaacaccacggccggctcgaccgtgacctactgggccaaccccgacgtctaccaccccgggcctgtgcagttttacatggcccgcgtgcccgatggcgaggacatcaactcgtggaacggcgacggcgccgtgtggttcaaggtgtacgaggaccatcctacctttggcgctcagctcacatggcccagcacgggcaagagctcgttcgcggttcccatccccccgtgcatcaagtccggctactacctcctccgggcggagcaaatcggcctgcacgtcgcccagagcgtaggcggagcgcagttctacatctcatgcgcccagctcagcgtcaccggcggcggcagcaccgagccgccgaacaaggtggccttccccggcgcttacagtgcgacggacccgggcattctgatcaacatctactaccctgttcccacgtcctaccagaaccccggcccggccgtcttcagctgctga

SEQ ID NO: 68 - protein, 452 aa, Thielavia terrestris
MLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSCMLANGAIVFLAAALGVSGHYTWPRVNDGADWQQVRKADNWQDNGYVGDVTSPQIRCFQATPSPAPSVLNTTAGSTVTYWANPDVYHPGPVQFYMARVPDGEDINSWNGDGAVWFKVYEDHPTFGAQLTWPSTGKSSFAVPIPPCIKSGYYLLRAEQIGLHVAQSVGGAQFYISCAQLSVTGGGSTEPPNKVAFPGAYSATDPGILINIYYPVPTSYQNPGPAVFSC

SEQ ID NO: 69 - DNA, 960 bp, Thielavia terrestris
atgaagggacttttcagtgccgccgccctctccctggccgtcggccaggcttcggcccattacatcttccagcaactctccatcaacgggaaccagtttccggtgtaccaatatattcgcaagaacaccaattataacagtcccgttaccgatctcacgtccgacgatcttcggtgcaatgtcggcgcccagggtgctgggacagacaccgtcacggtgaaggccggcgaccagttcaccttcacccttgacacccctgtttaccaccaggggcccatctccatctacatgtccaaggccccgggcgcggcgtcagactacgatggcagcggcggctggttcaagatcaaggactggggcccgactttcaacgccgacggcacggccacctgggacatggccggctcatacacctacaacatcccgacctgcattcccgacggcgactatctgctccgcatccagtcgctggccatccacaacccctggccggcgggcatcccgcagttctacatctcctgcgcccagatcaccgtgaccggcggcggcaacggcaaccctggcccgacggccctcatccccggcgccttcaaggacaccgacccgggctacacggtgaacatctacacgaacttccacaactacacggttcccggcccggaggtcttcagctgcaacggcggcggctcgaacccgcccccgccggtgagtagcagcacgcccgcgaccacgacgctggtcacgtcgacgcgcaccacgtcctccacgtcctccgcctcgacgccggcctcgaccggcggctgcaccgtcgccaagtggggccagtgcggcggcaacgggtacaccggctgcacgacctgcgcggccgggtccacctgcagcaagcagaacgactactactcgcagtgcttgtaagggaggccgcaaagcatgaggtgtttgaagaggaggagaggggtc

SEQ ID NO: 70 - protein, 608 aa, Thielavia terrestris
MKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCLMKGLFSAAALSLAVGQASAHYIFQQLSINGNQFPVYQYIRKNTNYNSPVTDLTSDDLRCNVGAQGAGTDTVTVKAGDQFTFTLDTPVYHQGPISIYMSKAPGAASDYDGSGGWFKIKDWGPTFNADGTATWDMAGSYTYNIPTCIPDGDYLLRIQSLAIHNPWPAGIPQFYISCAQITVTGGGNGNPGPTALIPGAFKDTDPGYTVNIYTNFHNYTVPGPEVFSCNGGGSNPPPPVSSSTPATTTLVTSTRTTSSTSSASTPASTGGCTVAKWGQCGGNGYTGCTTCAAGSTCSKQNDYYSQCL

SEQ ID NO: 71 - DNA, 954 bp, Thielavia terrestris
atgaagggcctcagcctcctcgccgctgcgtcggcagcgactgctcataccatcttcgtgcagctcgagtcagggggaacgacctatccggtatcctacggcatccgggaccctagctacgacggtcccatcaccgacgtcacctccgactcactggcttgcaatggtcccccgaaccccacgacgccgtccccgtacatcatcaacgtcaccgccggcaccacggtcgcggcgatctggaggcacaccctcacatccggccccgacgatgtcatggacgccagccacaaggggccgaccctggcctacctcaagaaggtcgatgatgccttgaccgacacgggtatcggcggcggctggttcaagatccaggaggccggttacgacaatggcaattgggctaccagcacggtgatcaccaacggtggcttccaatatattgacatccccgcctgcattcccaacggccagtatctgctccgcgccgagatgatcgcgctccacgccgccagcacgcagggtggtgcccagctctacatggagtgcgcgcagatcaacgtggtgggcggctccggcagcgccagcccgcagacgtacagcatcccgggcatctaccaggcaaccgacccgggcctgctgatcaacatctactccatgacgccgtccagccagtacaccattccgggtccgcccctgttcacctgcagcggcagcggcaacaacggcggcggcagcaacccgtcgggcgggcagaccacgacggcgaagcccacgacgacgacggcggcgacgaccacctcctccgccgctcctaccagcagccaggggggcagcagcggttgcaccgttccccagtggcagcagtgcggtggcatctcgttcaccggctgcaccacctgcgcggcgggctacacctgcaagtatctgaacgactattactcgcaatgccagtaa

SEQ ID NO: 72 - protein, 317 aa, Thielavia terrestris
MKGLSLLAAASAATAHTIFVQLESGGTTYPVSYGIRDPSYDGPITDVTSDSLACNGPPNPTTPSPYIINVTAGTTVAAIWRHTLTSGPDDVMDASHKGPTLAYLKKVDDALTDTGIGGGWFKIQEAGYDNGNWATSTVITNGGFQYIDIPACIPNGQYLLRAEMIALHAASTQGGAQLYMECAQINVVGGSGSASPQTYSIPGIYQATDPGLLINIYSMTPSSQYTIPGPPLFTCSGSGNNGGGSNPSGGQTTTAKPTTTTAATTTSSAAPTSSQGGSSGCTVPQWQQCGGISFTGCTTCAAGYTCKYLNDYYSQCQ

SEQ ID NO: 73 - DNA, 799 bp, Thermoascus aurantiacus
atgtccttttccaagataattgctactgccggcgttcttgcctctgcttctctagtggctggccatggcttcgttcagaacatcgtgattgatggtaaaaagtatgtcattgcaagacgcacataagcggcaacagctgacaatcgacagttatggcgggtatctagtgaaccagtatccatacatgtccaatcctccagaggtcatcgcctggtctactacggcaactgatcttggatttgtggacggtactggataccaaaccccagatatcatctgccataggggcgccaagcctggagccctgactgctccagtctctccaggaggaactgttgagcttcaatggactccatggcctgattctcaccatggcccagttatcaactaccttgctccgtgcaatggtgattgttccactgtggataagacccaattagaattcttcaaaattgccgagagcggtctcatcaatgatgacaatcctcctgggatctgggcttcagacaatctgatagcagccaacaacagctggactgtcaccattccaaccacaattgcacctggaaactatgttctgaggcatgagattattgctcttcactcagctcagaaccaggatggtgcccagaactatccccagtgcatcaatctgcaggtcactggaggtggttctgataaccctgctggaactcttggaacggcactctaccacgataccgatcctggaattctgatcaacatctatcagaaactttccagctatatcatccctggtcctcctctgtatactggttaa

SEQ ID NO: 74 - protein, 250 aa, Thermoascus aurantiacus
MSFSKIIATAGVLASASLVAGHGFVQNIVIDGKKYYGGYLVNQYPYMSNPPEVIAWSTTATDLGFVDGTGYQTPDIICHRGAKPGALTAPVSPGGTVELQWTPWPDSHHGPVINYLAPCNGDCSTVDKTQLEFFKIAESGLINDDNPPGIWASDNLIAANNSWTVTIPTTIAPGNYVLRHEIIALHSAQNQDGAQNYPQCINLQVTGGGSDNPAGTLGTALYHDTDPGILINIYQKLSSYIIPGPPLYTG

SEQ ID NO: 75 - DNA, 1172 bp, Trichoderma reesei
ggatctaagccccatcgatatgaagtcctgcgccattcttgcagcccttggctgtcttgccgggagcgttctcggccatggacaagtccaaaacttcacgatcaatggacaatacaatcagggtttcattctcgattactactatcagaagcagaatactggtcacttccccaacgttgctggctggtacgccgaggacctagacctgggcttcatctcccctgaccaatacaccacgcccgacattgtctgtcacaagaacgcggccccaggtgccatttctgccactgcagcggccggcagcaacatcgtcttccaatggggccctggcgtctggcctcacccctacggtcccatcgttacctacgtggctgagtgcagcggatcgtgcacgaccgtgaacaagaacaacctgcgctgggtcaagattcaggaggccggcatcaactataacacccaagtctgggcgcagcaggatctgatcaaccagggcaacaagtggactgtgaagatcccgtcgagcctcaggcccggaaactatgtcttccgccatgaacttcttgctgcccatggtgcctctagtgcgaacggcatgcagaactatcctcagtgcgtgaacatcgccgtcacaggctcgggcacgaaagcgctccctgccggaactcctgcaactcagctctacaagcccactgaccctggcatcttgttcaacccttacacaacaatcacgagctacaccatccctggcccagccctgtggcaaggctagatccaggggtacggtgttggcgttcgtgaagtcggagctgttgacaaggatatctgatgatgaacggagaggactgatgggcgtgactgagtgtatatatttttgatgaccaaattgtatacgaaatccgaacgcatggtgatcattgtttatccctgtagtatattgtctccaggctgctaagagcccaccgggtgtattacggcaacaaagtcaggaatttgggtggcaatgaacgcaggtctccatgaatgtatatgtgaagaggcatcggctggcatgggcattaccagatataggccctgtgaaacatatagtacttgaacgtgctactggaacggatcataagcaagtcatcaacatgtgaaaaaacactacatgtaaaaaaaaaaaaaaaaaaaaa

SEQ ID NO: 76 - protein, 249 aa, Trichoderma reesei
MKSCAILAALGCLAGSVLGHGQVQNFTINGQYNQGFILDYYYQKQNTGHFPNVAGWYAEDLDLGFISPDQYTTPDIVCHKNAAPGAISATAAAGSNIVFQWGPGVWPHPYGPIVTYVVECSGSCTTVNKNNLRWVKIQEAGINYNTQVWAQQDLINQGNKWTVKIPSSLRPGNYVFRHELLAAHGASSANGMQNYPQCVNIAVTGSGTKALPAGTPATQLYKPTDPGILFNPYTTITSYTIPGPALWQG

SEQ ID NO: 77 - DNA, 38 bp, Trichoderma reesei
actggatttaccatgaacaagtccgtggctccattgct

SEQ ID NO: 78 - DNA, 38 bp, Trichoderma reesei
tcacctctagttaattaactactttcttgcgagacacg

SEQ ID NO: 79 - DNA, 29 bp, Trichoderma reesei
aacgttaattaaggaatcgttttgtgttt

SEQ ID NO: 80 - DNA, 29 bp, Trichoderma reesei
agtactagtagctccgtggcgaaagcctg

SEQ ID NO: 81 - DNA, 31 bp, Saccharomyces cerevisiae
ttgaattgaaaatagattgatttaaaacttc

SEQ ID NO: 82 - DNA, 25 bp, Saccharomyces cerevisiae
ttgcatgcgtaatcatggtcatagc

SEQ ID NO: 83 - DNA, 26 bp, Saccharomyces cerevisiae
ttgaattcatgggtaataactgatat

SEQ ID NO: 84 - DNA, 32 bp, Saccharomyces cerevisiae
aaatcaatctattttcaattcaattcatcatt

SEQ ID NO: 85 - DNA, 11 bp, Saccharomyces cerevisiae
gtactaaaacc

SEQ ID NO: 86 - DNA, 11 bp, Saccharomyces cerevisiae
ccgttaaattt

SEQ ID NO: 87 - DNA, 45 bp, Saccharomyces cerevisiae
ggatgctgttgactccggaaatttaacggtttggtcttgcatccc

SEQ ID NO: 88 - DNA, 14 bp, Saccharomyces cerevisiae
atgcaatttaaact

SEQ ID NO: 89 - DNA, 14 bp, Saccharomyces cerevisiae
cggcaatttaacgg

SEQ ID NO: 90 - DNA, 44 bp, Saccharomyces cerevisiae
ggtattgtcctgcagacggcaatttaacggcttctgcgaatcgc

SEQ ID NO: 91 - DNA, 29 bp, Humicola insolens
aagcttaagcatgcgttcctcccccctcc

SEQ ID NO: 92 - DNA, 32 bp, Humicola insolens
ctgcagaattctacaggcactgatggtaccag

SEQ ID NO: 93 - DNA, 32 bp, Trichoderma reesei; identical to SEQ ID NO: 92

SEQ ID NO: 94 - DNA, 36 bp, Trichoderma reesei
accgcggactgcgcatcatgcgttcctcccccctcc

SEQ ID NO: 95 - DNA, 29 bp, Trichoderma reesei
aaacgtcgaccgaatgtaggattgttatc

SEQ ID NO: 96 - DNA, 17 bp, Trichoderma reesei
gatgcgcagtccgcggt

SEQ ID NO: 97 - DNA, 29 bp, Trichoderma reesei
aaacgtcgaccgaatgtaggattgttatc

SEQ ID NO: 98 - DNA, 36 bp, Trichoderma reesei
ggaggggggaggaacgcatgatgcgcagtccgcggt

SEQ ID NO: 99 - DNA, 29 bp, Trichoderma reesei
aaacgtcgaccgaatgtaggattgttatc

SEQ ID NO: 100 - DNA, 32 bp, Trichoderma reesei; identical to SEQ ID NO: 92

SEQ ID NO: 101 - DNA, 46 bp, Aspergillus oryzae
atagtcaaccgcggactgcgcatcatgaagcttggttggatcgagg

SEQ ID NO: 102 - DNA, 26 bp, Aspergillus oryzae
actagtttactgggccttaggcagcg

SEQ ID NO: 103 - DNA, 26 bp, Trichoderma reesei
gtcgactcgaagcccgaatgtaggat

SEQ ID NO: 104 - DNA, 45 bp, Trichoderma reesei
cctcgatccaaccaagcttcatgatgcgcagtccgcggttgacta

SEQ ID NO: 105 - DNA, 57 bp, Aspergillus oryzae
atgaagcttggttggatcgaggtggccgcattggcggctgcctcagtagtcagtgcc

SEQ ID NO: 106 - protein, 19 aa, Aspergillus oryzae
MKLGWIEVAALAAASVVSA

SEQ ID NO: 107 - DNA, 42 bp, Aspergillus oryzae
tgccggtgttggcccttgccaaggatgatctcgcgtactccc

SEQ ID NO: 108 - DNA, 28 bp, Aspergillus oryzae
gactagtcttactgggccttaggcagcg

SEQ ID NO: 109 - DNA, 63 bp, Humicola insolens
atgcgttcctcccccctcctccgctccgccgttgtggccgccctgccggtgttggcccttgcc

SEQ ID NO: 110 - protein, 21 aa, Humicola insolens
MRSSPLLRSAVVAALPVLALA

SEQ ID NO: 111 - DNA, 30 bp, Aspergillus oryzae
acgcgtcgaccgaatgtaggattgttatcc

SEQ ID NO: 112 - DNA, 42 bp, Aspergillus oryzae
gggagtacgcgagatcatccttggcaagggccaacaccggca

SEQ ID NO: 113 - DNA, 2586 bp, Aspergillus oryzae; identical to SEQ ID NO: 47

SEQ ID NO: 114 - protein, 861 aa, Aspergillus oryzae; identical to SEQ ID NO: 48

SEQ ID NO: 115 - DNA, 20 bp, Trichoderma reesei
cccaagcttagccaagaaca

SEQ ID NO: 116 - DNA, 29 bp, Trichoderma reesei
gggggaggaacgcatgggatctggacggc

SEQ ID NO: 117 - DNA, 30 bp, Aspergillus oryzae
gccgtccagatccccatgcgttcctccccc

SEQ ID NO: 118 - DNA, 20 bp, Aspergillus oryzae
ccaagcttgttcagagtttc

SEQ ID NO: 119 - DNA, 20 bp, Aspergillus oryzae
ggactgcgcagcatgcgttc

SEQ ID NO: 120 - DNA, 30 bp, Aspergillus oryzae
agttaattaattactgggccttaggcagcg

SEQ ID NO: 121 - DNA, 28 bp, Thermoascus aurantiacus
atgtccttttccaagataattgctactg

SEQ ID NO: 122 - DNA, 26 bp, Thermoascus aurantiacus
gcttaattaaccagtatacagaggag

(19) United States
(12) Patent Application Publication
(10) Pub. No.: US 2009/0123979 A1
(43) Pub. Date: May 14, 2009

(54) Methods of reducing the inhibitory effect of a tannin on the enzymatic hydrolysis of cellulosic material

(75) Inventor: Feng Xu, Davis, CA (US)

Correspondence Address: NOVOZYMES, INC., 1445 DREW AVE, DAVIS, CA, 95616, US

(73) Assignee: Novozymes, Inc., Davis, CA (US)

(21) Appl. No.: 12/262,738

(22) Filed: Oct. 31, 2008

Related U.S. Application Data
(60) Provisional application No. 60/984,627, filed on Nov. 1, 2007.

(51) Int. Cl. C12P 19/04

(52) U.S. Cl. 435/101

(57) ABSTRACT

The present invention relates to methods of producing a cellulosic material reduced in a tannin, comprising treating the cellulosic material with an effective amount of a tannase to reduce the inhibitory effect of the tannin on enzymatically saccharifying the cellulosic material. The present invention also relates to methods of saccharifying a cellulosic material, comprising: treating the cellulosic material with an effective amount of a tannase and an effective amount of a cellulolytic enzyme composition, wherein the treating of the cellulosic material with the tannase reduces the inhibitory effect of a tannin on enzymatically saccharifying the cellulosic material with the cellulolytic enzyme composition. The present invention also relates to methods of producing a fermentation product, comprising: (a) saccharifying a cellulosic material with an effective amount of a cellulolytic enzyme composition; (b) fermenting the saccharified cellulosic material of step (a) with one or more fermenting microorganisms to produce a fermentation product; and (c) recovering the fermentation product, wherein the cellulosic material is treated with an effective amount of a tannase to reduce the inhibitory effect of a tannin on enzymatically saccharifying the cellulosic material.

### CROSS-REFERENCE TO RELATED APPLICATION

This application claims the benefit of U.S. Provisional Application No. 60/984,627, filed Nov. 1, 2007, which application is incorporated herein by reference.

### REFERENCE TO A SEQUENCE LISTING

This application contains a Sequence Listing in computer readable form. The computer readable form is incorporated herein by reference.

### BACKGROUND OF THE INVENTION

1. Field of the Invention

The present invention relates to methods of reducing the inhibition of a cellulolytic enzyme composition by a tannin to improve the hydrolysis of a cellulosic material into fermentable sugars.

2. Description of the Related Art

Biomass feedstocks for the production of ethanol and other chemicals are complex in composition, comprising cellulose, hemicellulose, lignin, and other constituents. Among the other constituents are tannins. Conventionally, tannins are divided into two groups: hydrolyzable tannins and condensed tannins. Hydrolyzable tannins (also known as tannic acids or gallotannins) are made of poly-galloyl or ellagoyl esters of glucose or other polyols. Condensed tannins (also known as proanthocyanidins, leucoanthocyanidins, pycnogenols, or oligomeric proanthocyanidin complexes (OPCs)) are made of oligo/polymerized derivatives of catechin, epicatechin, flavonol, or other flavanoids.

It has been reported that tannins can form soluble or insoluble complexes with proteins (Zanobini et al., 1967, Experientia 23: 1015-1016; Oh et al., 1980, J. Agric. Food Chem. 28: 394-398). When the complexed protein is an enzyme, the tannin-protein interaction can lead to loss of enzymatic activity. Griffiths and Jones, 1977, J. Sci. Food Agric. 28: 983-989; Griffiths, 1981, J. Sci. Food Agric. 32: 797-804; and Kumar, 1992, Basic Life Sci. 59: 699-704, describe the inhibition of rumen (bacterial) cellulases by tannins.

The present invention relates to methods of reducing the inhibitory effect of a tannin on the enzymatic hydrolysis of a cellulosic material.

### SUMMARY OF THE INVENTION

The present invention relates to methods of producing a cellulosic material reduced in a tannin, comprising treating the cellulosic material with an effective amount of a tannase to reduce the inhibitory effect of the tannin on enzymatically saccharifying the cellulosic material.

The present invention also relates to methods of saccharifying a cellulosic material, comprising: treating the cellulosic material with an effective amount of a tannase and an effective amount of a cellulolytic enzyme composition, wherein the treating of the cellulosic material with the tannase reduces the inhibitory effect of a tannin on enzymatically saccharifying the cellulosic material with the cellulolytic enzyme composition.

The present invention also relates to methods of producing a fermentation product, comprising: (a) saccharifying a cellulosic material with an effective amount of a cellulolytic enzyme composition; (b) fermenting the saccharified cellulosic material of step (a) with one or more fermenting microorganisms to produce a fermentation product; and (c) recovering the fermentation product, wherein the cellulosic material is treated with an effective amount of a tannase to reduce the inhibitory effect of a tannin on enzymatically saccharifying the cellulosic material.

### BRIEF DESCRIPTION OF THE FIGURES

FIG. 1 shows a restriction map of pAILo27.

FIG. 2 shows a restriction map of pMJ04.

FIG. 3 shows a restriction map of pCaHj527.

FIG. 4 shows a restriction map of pMT2188.

FIG. 5 shows a restriction map of pCaHj568.

FIG. 6 shows a restriction map of pMJ05.

FIG. 7 shows a restriction map of pSMai130.

FIG. 8 shows the DNA sequence and deduced amino acid sequence of an Aspergillus oryzae beta-glucosidase native signal sequence (SEQ ID NOs: 105 and 106).

FIG. 9 shows the DNA sequence and deduced amino acid sequence of a Humicola insolens endoglucanase V signal sequence (SEQ ID NOs: 109 and 110).

FIG. 10 shows a restriction map of pSMai135.

FIG. 11 shows a restriction map of pSMai140.

FIG. 12 shows a restriction map of pSaMe-F1.

FIG. 13 shows a restriction map of pSaMe-FX.

FIG. 14 shows a restriction map of pAlLo47.

FIG. 15 shows a restriction map of pSaMe-FH.

FIGS. 16A and 16B show the effect of a mixture of tannic acid, ellagic acid, epicatechin, 4-hydroxyl-2-methylbenzoic acid, vanillin, coniferyl alcohol, coniferyl aldehyde, ferulic acid, and syringaldehyde (1 mM each) on the hydrolysis of PCS by Cellulolytic Enzyme Composition #1 (A) or Cellulolytic Enzyme Composition #2 (B) over 4 or 5 days. The hydrolysis reactions were conducted with 43 g of PCS and 0.25 g of Cellulolytic Enzyme Composition #1 or Cellulolytic Enzyme Composition #2 per liter of 50 mM sodium acetate pH 5 at 50° C.

FIGS. 17A, 17B, and 17C show the effect of tannic acid, 4-hydroxyl-2-methylbenzoic acid, vanillin, coniferyl alcohol, coniferyl aldehyde, ferulic acid, syringaldehyde, ellagic acid, or epicatechin (1 mM each) on PCS hydrolysis by Cellulolytic Enzyme Composition #1 (A and C) or Cellulolytic Enzyme Composition #2 (B) over 4 or 5 days. The hydrolysis reactions were conducted with 43 g of PCS and 0.25 g of Cellulolytic Enzyme Composition #1 or Cellulolytic Enzyme Composition #2 per liter of 50 mM sodium acetate pH 5 at 50° C.

FIGS. 18A and 18B show the effect of OPC (10 mM) or flavonol (1 mM) on PCS hydrolysis by Cellulolytic Enzyme Composition #1 (A) or Cellulolytic Enzyme Composition #2 (B) over 4 days. The hydrolysis reactions were conducted with 43 g of PCS and 0.25 g of Cellulolytic Enzyme Composition #1 or Cellulolytic Enzyme Composition #2 per liter of 50 mM sodium acetate pH 5 at 50° C.

FIGS. 19A, 19B, 19C, and 19D show the effective inhibitory concentration range of tannic acid (A and B) or OPC (C and D) on the hydrolysis of AVICEL® by Cellulolytic Enzyme Composition #1. The concentration of tannic acid ranged from 0.05 mM to 1 mM (A and B), while the concentration of OPC (in flavanone-equivalent subunits) ranged from 1 mM to 10 mM (C and D). The hydrolysis reactions were conducted with 23 g of AVICEL® and 0.25 g of Cellulolytic Enzyme Composition #1 per liter of 50 mM sodium acetate pH 5 at 50° C. Dixon plot: (B) for tannic acid, linear regression line: 1/Rate=(0.356±0.033)[tannic acid]+(0.045±0.017), r²=0.975; (D) for OPC, linear regression line: 1/Rate=(0.0070±0.0007)[OPC]+(0.056±0.004), r²=0.972. Rate estimated from the hydrolysis difference (%) at 0 and 6 hours.

FIGS. 20A, 20B, 20C, and 20D show the effective inhibitory concentration range for tannic acid or OPC on PCS hydrolysis by Cellulolytic Enzyme Composition #2. The concentration of tannic acid ranged from 0.1 mM to 1 mM (A and B), while the concentration of OPC ranged from 0.1 mM to 10 mM (C and D). The hydrolysis reactions were conducted with 43 g of PCS and 0.25 g of Cellulolytic Enzyme Composition #2 per liter of 50 mM sodium acetate pH 5 at 50° C. Dixon plot: (B) for tannic acid, linear regression line: 1/Rate=(0.098±0.009)[tannic acid]+(0.018±0.005), r²=0.983); (D) for OPC, linear regression line: 1/Rate=(0.0077±0.0004)[OPC]+(0.023±0.002), r²=0.996); the rate was estimated from the hydrolysis difference (%) at 0 and 5 hours.

FIGS. 21A, 21B, 21C, and 21D show the effect of 1 mM tannic acid on Trichoderma reesei CEL7A cellobiohydrolase I (CBHI) (A), Trichoderma reesei CEL6A cellobiohydrolase II (CBHII) (B), Trichoderma reesei CEL7B endoglucanase I (EGI) (C), and Trichoderma reesei CEL5A endoglucanase II (EGII) (D) hydrolysis of PASC over 4 hours. The hydrolysis reactions were conducted with 2 g of PASC and 40 mg of enzyme per liter of 50 mM sodium acetate pH 5 at 50° C.

FIGS. 22A and 22B show the inhibition of Trichoderma reesei CEL7B endoglucanase I (EGI) (A) and Trichoderma reesei CEL5A endoglucanase II (EGII) (B) by 1 mM tannic acid on the hydrolysis of carboxymethylcellulose (CMC) over 4 hours. The hydrolysis reactions were conducted with 10 g of CMC and 20 mg of CEL7B EGI or 10 mg of CEL5A EGII per liter of 50 mM sodium acetate pH 5 at 50° C.

FIG. 23 shows the effect of 1 mM tannic acid on cellobiose hydrolysis by Aspergillus oryzae CEL3A beta-glucosidase over 4 hours. The hydrolysis reactions were conducted with 2 g of cellobiose and 1 mg of beta-glucosidase per liter of 50 mM sodium acetate pH 5 at 50° C.

FIGS. 24A and 24B show the effect of an Aspergillus oryzae tannase on PCS hydrolysis by Cellulolytic Enzyme Composition #2 in the presence of 1 mM tannic acid (A) and 10 mM OPC (B) over 4 hours. The hydrolysis reactions were conducted with 43 g of PCS, 25 mg of tannase, and 0.25 g of Cellulolytic Enzyme Composition #2 per liter of 50 mM sodium acetate pH 5 at 50° C.

FIG. 25 shows the effect of Aspergillus oryzae tannase on PCS hydrolysis by Cellulolytic Enzyme Composition #1 in the presence of tannic acid. The hydrolysis reactions were conducted with 43.4 g of PCS and 0.25 g of Cellulolytic Enzyme Composition #1 per liter of 50 mM sodium acetate pH 5 at 50° C. for up to 4 days. Hydrolysis profiles. Symbol: (◯) no tannic acid, no tannase, (Δ) 1 mM tannic acid, (x) 1 mM tannic acid, 12.5 mg of tannase per liter, (+) 1 mM tannic acid, 25 mg/L tannase, (−) 1 mM tannic acid, 50 mg of tannase per liter.

### DEFINITIONS

Tannin: The term “tannin” is defined herein as a compound of Mᵣ 500-20,000, containing a sufficient number of phenolic hydroxyl groups (about 2 groups per Mᵣ 100) to form cross-links or other interactions with macromolecules, such as proteins, cellulose, and/or pectin, as well as alkaloids. There are two classes of tannins: hydrolyzable tannins and condensed tannins. In one aspect, the tannin is a hydrolyzable tannin, a condensed tannin, or a combination thereof.

Hydrolyzable Tannins: The term “hydrolyzable tannins” is defined herein as tannins that can be hydrolyzed to glucose (or another polyhydric alcohol) and gallic acid (gallotannins) or ellagic (ellagitannins). The simplest known gallotannin is 1-O-galloyl-beta-D-glucopyranose. In contrast, gallotannin (tannic acid) contains up to 10 galloyl groups. Ellagotannins are derivatives of hexahydroxydiphenic acid, which becomes lactonized to ellagic acid during hydrolysis. The simplest known ellagitannin is corilagin.

Condensed Tannins: The term “condensed tannins” is defined herein as polymers in which the monomeric unit is a phenolic flavovoid, usually a flavonol, and in which flavonoid units are linked by 4:8 (C—C) bonds. Condensed tannins are also known as proanthocyanidins, leucoanthocyanidins, pycnogenols, or oligomeric proanthocyanidin complexes (OPC).

Tannic Acid: The term “tannic acid” is defined herein as a gallotannin, which contains up to 10 galloyl groups.

Gallic Acid: The term “gallic acid” is defined herein as 3,4,5-trihydroxybenzoic acid. Salts and esters of gallic acid are known as gallates.

Oligomeric Proanthocyanidin Complexes (OPC): The term “oligomeric proanthocyanidin complexes” is defined herein as a class of flavonoid complexes.

Tannase: The term “tannase” is defined herein as a tannin acylhydrolase (EC 3.1.1.20) that catalyzes the hydrolysis of a tannin (such as gallotannin) to a phenolic acid and a carbohydrate (such as gallic acid and glucose) (see Schomburg and Schomburg, 2003, Springer Handbook of Enzymes, Springer, pp 187-190). Tannase can be assayed by following detection of gallic acid from methyl gallate, a surrogate substrate of gallotannin (tannic acid) under specified conditions of pH and temperature. One unit (U) of tannase activity equals the amount of enzyme capable of releasing 1 micromole of gallic acid produced per minute at a specified pH and temperature (° C.). For example, a reaction solution of 0.5 ml containing tannase and 5 mM methyl gallate in 50 mM sodium citrate pH 5 is incubated at 30° C. for 5 minutes. Then 0.3 ml of 0.667% (w/v) rhodanine dissolved in methanol is added, and the mixture is incubated at 30° C. for 5 minutes. Then, 0.2 ml of 0.5 M KOH is added, and the mixture is incubated at 30° C. for 2.5 minutes. Finally, 4 ml of water is added, and the mixture is incubated at 30° C. for 10 minutes, and the absorbance is recorded at 520 nm. Mixtures omitting either tannase, methyl gallate, or rhodanine serve as controls. Gallic acid is used as standard for calibration. The specific activity of tannase is expressed in units of micromole of gallic acid produced per minute per mg of tannase at pH 5 and 30° C. See Sharma et al., 1999, World Journal of Microbiology and Biotechnology 15(6), 673-677.

Cellulolytic activity: The term “cellulolytic activity” is defined herein as a biological activity that hydrolyzes a cellulose-containing material. Cellulolytic protein may hydrolyze filter paper (FP), thereby decreasing the mass of insoluble paper and increasing the amount of soluble sugars. The reaction can be measured by detection of reducing sugars that forms colored products with p-hydroxybenzoic acid hydrazide, determined in terms of Filter Paper Assay Unit (FPU). Cellulolytic protein may hydrolyze microcrystalline celluose or other cellulosic substances, thereby decreasing the mass of insoluble cellulose and increasing the amount of soluble sugars. The reaction can be measured by the detection of reducing sugars with p-hydroxybenzoic acid hydrazide, a high-performance-liquid-chromatography (HPLC), or an electrochemical sugar detector. Cellulolytic protein may hydrolyze soluble, chromogenic, fluorogenic, or other like glycoside substances, thereby increasing the amount of chromophoric, fluorophoric, or other physically-detectable products. The reaction may be monitored using a spectrophotometer, fluorometer, or other instrument. Cellulolytic protein may hydrolyze carboxymethyl cellulose (CMC), thereby decreasing the viscosity of the incubation mixture. The resulting reduction in viscosity may be determined by a vibration viscosimeter (e.g., MIVI 3000 from Sofraser, France). Determination of cellulase activity, measured in terms of Cellulase Viscosity Unit (CEVU), quantifies the amount of catalytic activity present in a sample by measuring the ability of the sample to reduce the viscosity of a solution of carboxymethyl cellulose (CMC). The assay is performed at a temperature and pH suitable for the cellulolytic protein and substrate. For example, for CELLUCLAST™ (Novozymes A/S, Bagsværd, Denmark) the assay is carried out at 40° C. in 0.1 M phosphate pH 9.0 buffer for 30 minutes with CMC as substrate (33.3 g/liter carboxymethyl cellulose Hercules 7 LFD) and an enzyme concentration of approximately 3.3-4.2 CEVU/ml. The CEVU activity is calculated relative to a declared enzyme standard, such as CELLUZYME™ Standard 17-1194 (obtained from Novozymes A/S, Bagsværd, Denmark).

For purposes of the present invention, cellulolytic activity is determined by measuring the increase in hydrolysis of a cellulosic material by a cellulolytic enzyme composition under the following conditions: 1-10 mg of cellulolytic protein/g of cellulose in PCS for 5-7 days at 50° C. compared to a control hydrolysis without addition of cellulolytic protein.

Endoglucanase: The term “endoglucanase” is defined herein as an endo-1,4-(1,3;1,4)-beta-D-glucan 4-glucanohydrolase (E.C. No. 3.2.1.4), which catalyses endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. For purposes of the present invention, endoglucanase activity is determined using carboxymethyl cellulose (CMC) hydrolysis according to the procedure of Ghose, 1987, Pure and Appl. Chem. 59: 257-268.

Cellobiohydrolase: The term “cellobiohydrolase” is defined herein as a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain. For purposes of the present invention, cellobiohydrolase activity is determined according to the procedures described by Lever et al., 1972, Anal. Biochem. 47: 273-279 and by van Tilbeurgh et al., 1982, FEBS Letters 149: 152-156; van Tilbeurgh and Claeyssens, 1985, FEBS Letters 187: 283-288.

Beta-glucosidase: The term “beta-glucosidase” is defined herein as a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the procedure described by Venturi et al., 2002, J. Basic Microbiol. 42: 55-66. One unit of beta-glucosidase activity is defined as 1.0 μmole of p-nitrophenol produced per minute at 50° C., pH 5 from 4 mM p-nitrophenyl-beta-D-glucopyranoside as substrate in 100 mM sodium citrate, 0.01% TWEEN® 20.

Cellulolytic enhancing activity: The term “cellulolytic enhancing activity” is defined herein as a biological activity of a GH61 polypeptide that enhances the hydrolysis of a cellulosic material by proteins having cellulolytic activity. For purposes of the present invention, cellulolytic enhancing activity is determined by measuring the increase in reducing sugars or the increase of the total of cellobiose and glucose from the hydrolysis of a cellulosic material by cellulolytic protein under the following conditions: 1-50 mg of total protein/g of cellulose in PCS, wherein total protein is comprised of 80-99.5% w/w cellulolytic protein/g of cellulose in PCS and 0.5-20% w/w protein of cellulolytic enhancing activity for 1-7 days at 50° C. compared to a control hydrolysis with equal total protein loading without cellulolytic enhancing activity (1-50 mg of cellulolytic protein/g of cellulose in PCS).

A GH61 polypeptide having cellulolytic enhancing activity enhances the hydrolysis of a cellulosic material catalyzed by proteins having cellulolytic activity by reducing the amount of cellulolytic enzyme required to reach the same degree of hydrolysis preferably at least 0.1-fold, more at least 0.2-fold, more preferably at least 0.3-fold, more preferably at least 0.4-fold, more preferably at least 0.5-fold, more preferably at least 1-fold, more preferably at least 3-fold, more preferably at least 4-fold, more preferably at least 5-fold, more preferably at least 10-fold, more preferably at least 20-fold, even more preferably at least 30-fold, most preferably at least 50-fold, and even most preferably at least 100-fold.

Family 61 glycoside hydrolase: The term “Family 61 glycoside hydrolase” or “Family GH61” is defined herein as a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. Presently, Henrissat lists the GH61 Family as unclassified indicating that properties such as mechanism, catalytic nucleophile/base, catalytic proton donors, and 3-D structure are not known for polypeptides belonging to this family.

Cellulosic material: The predominant polysaccharide in the primary cell wall of biomass is cellulose, the second most abundant is hemi-cellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

The cellulosic material can be any material containing cellulose. Cellulose is generally found, for example, in the stems, leaves, hulls, husks, and cobs of plants or leaves, branches, and wood of trees. The cellulosic material can be, but is not limited to, herbaceous material, agricultural residue, forestry residue, municipal solid waste, waste paper, and pulp and paper mill residue The cellulosic material can be any type of biomass including, but not limited to, wood resources, municipal solid waste, wastepaper, crops, and crop residues (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix.

In one aspect, the cellulosic material is herbaceous material. In another aspect, the cellulosic material is agricultural residue. In another aspect, the cellulosic material is forestry residue. In another aspect, the cellulosic material is municipal solid waste. In another aspect, the cellulosic material is waste paper. In another aspect, the cellulosic material is pulp and paper mill residue.

In another aspect, the cellulosic material is corn stover. In another preferred aspect, the cellulosic material is corn fiber. In another aspect, the cellulosic material is corn cob. In another aspect, the cellulosic material is orange peel. In another aspect, the cellulosic material is rice straw. In another aspect, the cellulosic material is wheat straw. In another aspect, the cellulosic material is switch grass. In another aspect, the cellulosic material is miscanthus. In another aspect, the cellulosic material is bagasse.

The cellulosic material may be used as is or may be subjected to pretreatment, using conventional methods known in the art. For example, physical pretreatment techniques can include various types of milling, irradiation, steaming/steam explosion, and hydrothermolysis; chemical pretreatment techniques can include dilute acid, alkaline, organic solvent, ammonia, sulfur dioxide, carbon dioxide, and pH-controlled hydrothermolysis; and biological pretreatment techniques can involve applying lignin-solubilizing microorganisms (see, for example, Hsu, T.-A., 1996, Pretreatment of biomass, in Handbook on Bioethanol; Production and Utilization, Wyman, C. E., ed., Taylor & Francis, Washington, D.C., 179-212; Ghosh, P., and Singh, A., 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N.J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson, L., and Hahn-Hagerdal, B., 1996, Fermentation of lignocellulosic hydrolysates for ethanol production, Enz. Microb. Tech. 18: 312-331; and Vallander, L., and Eriksson, K.-E. L., 1990, Production of ethanol from lignocellulosic materials: State of the art, Adv. Biochem. Eng./Biotechnol. 42: 63-95).

Pretreated corn stover: The term “PCS” or “Pretreated Corn Stover” is defined herein as a cellulosic material derived from corn stover by treatment with heat and dilute acid. For purposes of the present invention, PCS is made by the method described in Example 26, or variations thereof in time, temperature and amount of acid.

Isolated polypeptide: The term “isolated polypeptide” as used herein refers to a polypeptide that is isolated from a source. In a preferred aspect, the polypeptide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by SDS-PAGE. For purposes of the present invention, the term “polypeptide” will be understood to include a full-length polypeptide, mature polypeptide, or catalytic domain; or portions or fragments thereof that have enzyme activity.

Substantially pure polypeptide: The term “substantially pure polypeptide” denotes herein a polypeptide preparation that contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polypeptide material with which it is natively or recombinantly associated. It is, therefore, preferred that the substantially pure polypeptide is at least 92% pure, preferably at least 94% pure, more preferably at least 95% pure, more preferably at least 96% pure, more preferably at least 96% pure, more preferably at least 97% pure, more preferably at least 98% pure, even more preferably at least 99%, most preferably at least 99.5% pure, and even most preferably 100% pure by weight of the total polypeptide material present in the preparation. The polypeptide is preferably in a substantially pure form, i.e., that the polypeptide preparation is essentially free of other polypeptide material with which it is natively or recombinantly associated. This can be accomplished, for example, by preparing the polypeptide by well-known recombinant methods or by classical purification methods.

Isolated polynucleotide: The term “isolated polynucleotide” as used herein refers to a polynucleotide that is isolated from a source. In a preferred aspect, the polynucleotide is at least 1% pure, preferably at least 5% pure, more preferably at least 10% pure, more preferably at least 20% pure, more preferably at least 40% pure, more preferably at least 60% pure, even more preferably at least 80% pure, and most preferably at least 90% pure, as determined by agarose electrophoresis.

Substantially pure polynucleotide: The term “substantially pure polynucleotide” as used herein refers to a polynucleotide preparation free of other extraneous or unwanted nucleotides and in a form suitable for use within genetically engineered protein production systems. Thus, a substantially pure polynucleotide contains at most 10%, preferably at most 8%, more preferably at most 6%, more preferably at most 5%, more preferably at most 4%, more preferably at most 3%, even more preferably at most 2%, most preferably at most 1%, and even most preferably at most 0.5% by weight of other polynucleotide material with which it is natively or recombinantly associated. A substantially pure polynucleotide may, however, include naturally occurring 5′ and 3′ untranslated regions, such as promoters and terminators. It is preferred that the substantially pure polynucleotide is at least 90% pure, preferably at least 92% pure, more preferably at least 94% pure, more preferably at least 95% pure, more preferably at least 96% pure, more preferably at least 97% pure, even more preferably at least 98% pure, most preferably at least 99%, and even most preferably at least 99.5% pure by weight. The polynucleotide is preferably in a substantially pure form, i.e., that the polynucleotide preparation is essentially free of other polynucleotide material with which it is natively or recombinantly associated. The polynucleotides may be of genomic, cDNA, RNA, semisynthetic, synthetic origin, or any combinations thereof.

cDNA: The term “cDNA” is defined herein as a DNA molecule that can be prepared by reverse transcription from a mature, spliced, mRNA molecule obtained from a eukaryotic cell. cDNA lacks intron sequences that may be present in the corresponding genomic DNA. The initial, primary RNA transcript is a precursor to mRNA that is processed through a series of steps before appearing as mature spliced mRNA. These steps include the removal of intron sequences by a process called splicing. cDNA derived from mRNA lacks, therefore, any intron sequences.

Nucleic acid construct: The term “nucleic acid construct” as used herein refers to a nucleic acid molecule, either single or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a coding sequence.

Control sequences: The term “control sequences” is defined herein to include all components necessary for the expression of a polynucleotide encoding a polypeptide. Each control sequence may be native or foreign to the nucleotide sequence encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a polypeptide.

Operably linked: The term “operably linked” denotes herein a configuration in which a control sequence is placed at an appropriate position relative to the coding sequence of a polynucleotide sequence such that the control sequence directs the expression of the coding sequence of a polypeptide.

Coding sequence: When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG and TGA. The coding sequence may be a DNA, cDNA, or recombinant nucleotide sequence.

Expression: The term “expression” includes any step involved in the production of a polypeptide including, but not limited to, transcription, post-transcriptional modification, translation, post-translational modification, and secretion.

Expression vector: The term “expression vector” is defined herein as a linear or circular DNA molecule that comprises a polynucleotide encoding a polypeptide and is operably linked to additional nucleotides that provide for its expression.

Host cell: The term “host cell”, as used herein, includes any cell type that is susceptible to transformation, transfection, transduction, and the like with a nucleic acid construct or expression vector comprising a polynucleotide.

### DETAILED DESCRIPTION OF THE INVENTION

The present invention relates to methods of reducing the inhibition of cellulolytic enzyme compositions by a tannin to improve the efficiency of enzymatic saccharification of a cellulosic material into fermentable sugars, which can then be converted by fermentation into a desired fermentation product. The production of the desired fermentation product from cellulosic material typically requires three major steps, which include pretreatment, enzymatic hydrolysis (saccharification), and fermentation.

The cellulosic material is preferably pretreated to reduce particle size, disrupt fiber walls, and expose carbohydrates of the cellulosic material, which increases the susceptibility of the cellulosic material carbohydrates to enzymatic hydrolysis. However, pretreatment also exposes tannins, which can inhibit the components of the cellulolytic enzyme composition during enzymatic hydrolysis of the carbohydrates. Moreover, during enzymatic hydrolysis of the carbohydrates, additional inhibitory tannin can be released, which can further inhibit the cellulolytic composition. Finally, the tannin can also have an adverse affect on the fermentation microorganism(s). The present invention, therefore, improves the efficiency of enzymatic saccharification of a cellulosic material into fermentable sugars and the conversion of the sugars into a desired fermentation product.

In one aspect, the present invention relates to methods of producing a cellulosic material reduced in a tannin, comprising treating the cellulosic material with an effective amount of a tannase to reduce the inhibitory effect of the tannin on enzymatically saccharifying the cellulosic material.

In another aspect, the present invention relates to methods of saccharifying a cellulosic material, comprising: treating the cellulosic material with an effective amount of a tannase and an effective amount of a cellulolytic enzyme composition, wherein the treating of the cellulosic material with the tannase reduces the inhibitory effect of a tannin on enzymatically saccharifying the cellulosic material with the cellulolytic enzyme composition.

In a further aspect, the present invention relates to methods of producing a fermentation product, comprising: (a) saccharifying a cellulosic material with an effective amount of a cellulolytic enzyme composition; (b) fermenting the saccharified cellulosic material of step (a) with one or more fermenting microorganisms to produce a fermentation product; and (c) recovering the fermentation product, wherein the cellulosic material is treated with an effective amount of a tannase to reduce the inhibitory effect of a tannin on enzymatically saccharifying the cellulosic material.

### Processing of Cellulosic Material

The methods of the present invention can be used to saccharify a cellulosic material, e.g., lignocellulose, to fermentable sugars and convert the fermentable sugars to many useful substances, e.g., chemicals and fuels. The production of a desired fermentation product from the cellulosic material typically involves pretreatment, enzymatic hydrolysis (saccharification), and fermentation.

The processing of the cellulosic material according to the present invention can be accomplished using processes conventional in the art. Moreover, the methods of the present invention may be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and cofermentation (SSCF); hybrid hydrolysis and fermentation (HHF); SHCF (separate hydrolysis and co-fermentation), HHCF (hybrid hydrolysis and fermentation), and direct microbial conversion (DMC). SHF uses separate process steps to first enzymatically hydrolyze the cellulosic material, e.g., lignocellulose, to fermentable sugars, e.g., glucose, cellobiose, cellotriose, and pentose sugars, and then ferment the fermentable sugars to ethanol. In SSF, the enzymatic hydrolysis of the cellulosic material, e.g., lignocellulose, and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the cofermentation of multiple sugars (Sheehan, J., and Himmel, M., 1999, Enzymes, energy and the environment: A strategic perspective on the U.S. Department of Energy's research and development activities for bioethanol, Biotechnol. Prog. 15: 817-827). HHF involves a separate hydrolysis separate step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, lignocellulose hydrolysis, and fermentation) in one or more steps where the same organism is used to produce the enzymes for conversion of the cellulosic material, e.g., lignocellulose, to fermentable sugars and to convert the fermentable sugars into a final product (Lynd, L. R., Weimer, P. J., van Zyl, W. H., and Pretorius, I. S., 2002, Microbial cellulose utilization: Fundamentals and biotechnology, Microbiol. Mol. Biol. Reviews 66: 506-577). It is understood herein that any method known in the art comprising pretreatment, enzymatic hydrolysis (saccharification), fermentation, or a combination thereof can be used in the practicing the methods of the present invention.

A conventional apparatus can include a fed-batch stirred reactor, a batch stirred reactor, a continuous flow stirred reactor with ultrafiltration, and/or a continuous plug-flow column reactor (Fernanda de Castilhos Corazza, Flávio Faria de Moraes, Gisella Maria Zanin and Ivo Neitzel, 2003, Optimal control in fed-batch reactor for the cellobiose hydrolysis, Acta Scientiarum. Technology 25: 33-38; Gusakov, A. V., and Sinitsyn, A. P., 1985, Kinetics of the enzymatic hydrolysis of cellulose: 1. A mathematical model for a batch reactor process, Enz. Microb. Technol. 7: 346-352), an attrition reactor (Ryu, S. K., and Lee, J. M., 1983, Bioconversion of waste cellulose by using an attrition bioreactor, Biotechnol. Bioeng. 25: 53-65), or a reactor with intensive stirring induced by an electromagnetic field (Gusakov, A. V., Sinitsyn, A. P., Davydkin, I. Y., Davydkin, V. Y., Protas, O. V., 1996, Enhancement of enzymatic cellulose hydrolysis using a novel type of bioreactor with intensive stirring induced by electromagnetic field, Appl. Biochem. Biotechnol. 56: 141-153). Additional reactor types include: Fluidized bed, upflow blanket, immobilized, and extruder type reactors for hydrolysis and/or fermentation.

The cellulosic material can be treated with a tannase before, during, and/or after pretreatment, during hydrolysis, and/or during fermentation. In a preferred aspect, the cellulosic material is treated with a tannase before pretreatment. In another preferred aspect, the cellulosic material is treated with a tannase during pretreatment. In another preferred aspect, the cellulosic material is treated with a tannase after pretreatment. In another preferred aspect, the cellulosic material is treated with a tannase before, during, and after pretreatment. In another preferred aspect, the cellulosic material is treated with a tannase during a combination of two or more of before, during, and after pretreatment. In another preferred aspect, the cellulosic material is treated with a tannase during hydrolysis. In another preferred aspect, the cellulosic material is treated with a tannase during fermentation. In another preferred aspect, the cellulosic material is treated with a tannase before, during, and after pretreatment, during hydrolysis, and during fermentation. In another preferred aspect, the cellulosic material is treated with a tannase during any combination of before, during, and after pretreatment, during hydrolysis, and during fermentation.

During tannase treatment, the pH is in the range of preferably about 2 to about 11, more preferably about 4 to about 8, and most preferably about 5 to about 6. The temperature is in the range of preferably about 20° C. to about 90° C., more preferably about 30° C. to about 70° C., and most preferably about 40° C. to about 60° C. The tannase is dosed in the range of preferably about 0.1 to about 10,000, more preferably about 1 to about 1000, and most preferably about 10 to about 100 units per g of dry cellulosic material.

Pretreatment. In practicing the methods of the present invention, any pretreatment process known in the art can be used to disrupt the plant cell wall components. The cellulosic material, e.g., lignocellulose, can also be subjected to pre-soaking, wetting, or conditioning prior to pretreatment using methods known in the art. Conventional pretreatments include, but are not limited to, steam pretreatment (with or without explosion), dilute acid pretreatment, hot water pretreatment, lime pretreatment, wet oxidation, wet explosion, ammonia fiber explosion, organosolv pretreatment, and biological pretreatment. Additional pretreatments include ultrasound, electroporation, microwave, supercritical CO₂, supercritical H₂O, and ammonia percolation pretreatments.

The cellulosic material can be pretreated before hydrolysis and/or fermentation. Pretreatment is preferably performed prior to the hydrolysis. Alternatively, the pretreatment can be carried out simultaneously with hydrolysis, such as simultaneously with treatment of the cellulosic material with one or more cellulolytic enzymes, or other enzyme activities, e.g., hemicellulases, to release fermentable sugars, such as glucose and/or maltose. In most cases the pretreatment step itself results in some conversion of biomass to fermentable sugars (even in absence of enzymes).

Steam Pretreatment. In steam pretreatment, the cellulosic material is heated to disrupt the plant cell wall components, including, for example, lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably done at 140-230° C., more preferably 160-200° C., and most preferably 170-190° C., where the optimal temperature range depends on any addition of a chemical catalyst. Residence time for the steam pretreatment is preferably 1-15 minutes, more preferably 3-12 minutes, and most preferably 4-10 minutes, where the optimal residence time depends on temperature range and any addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 20020164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

A catalyst such as H₂SO₄ or SO₂ (typically 0.3 to 3% w/w) is often added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762).

Chemical Pretreatment: The term “chemical treatment” refers to any chemical pretreatment that promotes the separation and/or release of cellulose, hemicellulose, and/or lignin. Examples of suitable chemical pretreatment processes include, for example, dilute acid pretreatment, lime pretreatment, wet oxidation, ammonia fiber/freeze explosion (AFEX), ammonia percolation (APR), and organosolv pretreatments.

In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technol. 91: 179-188; Lee et al., 1999, Adv. Biochem. Eng. Biotechnol. 65: 93-115).

Several methods of pretreatment under alkaline conditions can also be used. These alkaline pretreatments include, but are not limited to, lime pretreatment, wet oxidation, ammonia percolation (APR), and ammonia fiber/freeze explosion (AFEX).

Lime pretreatment is performed with calcium carbonate, sodium hydroxide, or ammonia at low temperatures of 85-150° C. and residence times from 1 hour to several days (Wyman et al., 2005, Bioresource Technol. 96: 1959-1966; Mosier et al., 2005, Bioresource Technol. 96: 673-686). WO 2006/110891, WO 2006/11899, WO 2006/11900, and WO 2006/110901 disclose pretreatment methods using ammonia.

Wet oxidation is a thermal pretreatment performed typically at 180-200° C. for 515 minutes with addition of an oxidative agent such as hydrogen peroxide or over-pressure of oxygen (Schmidt and Thomsen, 1998, Bioresource Technol. 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed at preferably 1-40% dry matter, more preferably 2-30% dry matter, and most preferably 5-20% dry matter, and often the initial pH is increased by the addition of alkali such as sodium carbonate.

A modification of the wet oxidation pretreatment method, known as wet explosion (combination of wet oxidation and steam explosion), can handle dry matter up to 30%. In wet explosion, the oxidizing agent is introduced during pretreatment after a certain residence time. The pretreatment is then ended by flashing to atmospheric pressure (WO 2006/032282).

Ammonia fiber explosion (AFEX) involves treating cellulosic material with liquid or gaseous ammonia at moderate temperatures such as 90-100° C. and high pressure such as 17-20 bar for 5-10 minutes, where the dry matter content can be as high as 60% (Gollapalli et al., 2002, Appl. Biochem. Biotechnol. 98: 23-35; Chundawat et al, 2007, Biotechnol. Bioeng. 96: 219-231; Alizadeh et al., 2005, Appl. Biochem. Biotechnol. 121:1133-1141; Teymouri et al., 2005, Bioresource Technol. 96: 20142018). AFEX pretreatment results in the depolymerization of cellulose and partial hydrolysis of hemicellulose. Lignin-carbohydrate complexes are cleaved.

Organosolv pretreatment delignifies cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121:219-230). Sulphuric acid is usually added as a catalyst. In organosolv pretreatment, the majority of the hemicellulose is removed.

Other examples of suitable pretreatment methods are described by Schell et al., 2003, Appl. Biochem. and Biotechnol. Vol. 105-108, p. 69-85, and Mosier et al., 2005, Bioresource Technology 96: 673686, and U.S. Published Application 2002/0164730.

In one aspect, the chemical pretreatment is preferably carried out as an acid treatment, and more preferably as a continuous dilute and/or mild acid treatment. The acid is typically sulfuric acid, but other acids can also be used, such as acetic acid, citric acid, nitric acid, phosphoric acid, tartaric acid, succinic acid, hydrogen chloride or mixtures thereof. Mild acid treatment is conducted in the pH range of preferably 1-5, more preferably 1-4, and most preferably 1-3. In one aspect, the acid concentration is in the range from preferably 0.01 to 20 wt % acid, more preferably 0.05 to 10 wt % acid, even more preferably 0.1 to 5 wt % acid, and most preferably 0.2 to 2.0 wt % acid. The acid is contacted with the cellulosic material and held at a temperature in the range of preferably 160-220° C., and more preferably 165-195° C., for periods ranging from seconds to minutes to, e.g., 1 second to 60 minutes.

In another aspect, pretreatment is carried out as an ammonia fiber explosion step (AFEX pretreatment step).

In another aspect, pretreatment takes place in an aqueous slurry. In preferred aspects, the cellulosic material is present during pretreatment in amounts preferably between 10-80 wt %, more preferably between 20-70 wt %, and most preferably between 30-60 wt %, such as around 50 wt %. The pretreated cellulosic material can be unwashed or washed using any method known in the art, e.g., washed with water.

Mechanical Pretreatment: The term “mechanical pretreatment” refers to various types of grinding or milling (e.g., dry milling, wet milling, or vibratory ball milling).

Physical Pretreatment: The term “physical pretreatment” refers to any pretreatment that promotes the separation and/or release of cellulose, hemicellulose, and/or lignin from lignocellulose-containing material. For example, physical pretreatment can involve irradiation (e.g., microwave irradiation), steaming/steam explosion, hydrothermolysis, and combinations thereof.

Physical pretreatment can involve high pressure and/or high temperature (steam explosion). In one aspect, high pressure means pressure in the range of preferably about 300 to about 600 psi, more preferably about 350 to about 550 psi, and most preferably about 400 to about 500 psi, such as around 450 psi. In another aspect, high temperature means temperatures in the range of about 100 to about 300° C., preferably about 140 to about 235° C. In a preferred aspect, mechanical pretreatment is performed in a batch-process, steam gun hydrolyzer system that uses high pressure and high temperature as defined above, e.g., a Sunds Hydrolyzer available from Sunds Defibrator AB, Sweden.

Combined Physical and Chemical Pretreatment: The cellulosic material can be pretreated both physically and chemically. For instance, the pretreatment step can involve dilute or mild acid treatment and high temperature and/or pressure treatment. The physical and chemical pretreatments can be carried out sequentially or simultaneously, as desired. A mechanical pretreatment can also be included.

Accordingly, in a preferred aspect, the cellulosic material is subjected to mechanical, chemical, or physical pretreatment, or any combination thereof to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the lignocellulose-containing material. Biological pretreatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Physicochemical and biological treatments for enzymatic/microbial conversion of lignocellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N.J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng./Biotechnol. 42: 63-95).

Saccharification. In the hydrolysis step, also known as saccharification, the pretreated cellulosic material is hydrolyzed to break down cellulose and alternatively also hemicellulose to fermentable sugars, such as glucose, xylose, xylulose, arabinose, maltose, mannose, galactose, or soluble oligosaccharides. In one aspect, the sugar is selected from the group consisting of glucose, xylose, mannose, galactose, arabinose, and cellobiose. The hydrolysis is performed enzymatically by a cellulolytic enzyme composition. The enzymes of the compositions can also be added sequentially.

Enzymatic hydrolysis is preferably carried out in a suitable aqueous environment under conditions that can be readily determined by one skilled in the art. In a preferred aspect, hydrolysis is performed under conditions suitable for the activity of the enzyme(s), i.e., optimal for the enzyme(s). The hydrolysis can be carried out as a fed batch or continuous process where the pretreated cellulosic material (substrate) is fed gradually to, for example, an enzyme containing hydrolysis solution.

The saccharification is generally performed in stirred-tank reactors or fermentors under controlled pH, temperature, and mixing conditions. Suitable process time, temperature, and pH conditions can readily be determined by one skilled in the art. For example, the saccharification can last up to 200 hours, but is typically performed for preferably about 12 to about 96 hours, more preferably about 16 to about 72 hours, and most preferably about 24 to about 48 hours. The temperature is in the range of preferably about 25° C. to about 80° C., more preferably about 30° C. to about 70° C., and most preferably about 40° C. to 60° C. The pH is in the range of preferably about 3 to about 8, more preferably about 3.5 to about 7, and most preferably about 4 to about 6, in particular about pH 5. The dry solids content is in the range of preferably about 5 to about 50 wt %, more preferably about 10 to about 40 wt %, and most preferably about 20 to about 30 wt %.

The cellulolytic enzyme composition preferably comprises enzymes having endoglucanase, cellobiohydrolase, and beta-glucosidase activities. In a preferred aspect, the cellulolytic enzyme composition further comprises one or more polypeptides having cellulolytic enhancing activity. In another preferred aspect, the cellulolytic enzyme preparation is supplemented with one or more additional enzyme activities selected from the group consisting of hemicellulases, esterases (e.g., lipases, phospholipases, and/or cutinases), proteases, laccases, peroxidases, or mixtures thereof. In the methods of the present invention, the additional enzyme(s) may be added prior to or during fermentation, including during or after propagation of the fermenting microorganism(s).

The enzymes may be derived or obtained from any suitable origin, including, bacterial, fungal, yeast, or mammalian origin. The term “obtained from” means herein that the enzyme may have been isolated from an organism that naturally produces the enzyme as a native enzyme. The term “obtained from” also means herein that the enzyme may have been produced recombinantly in a host organism employing methods described herein, wherein the recombinantly produced enzyme is either native or foreign to the host organism or has a modified amino acid sequence, e.g., having one or more amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

The enzymes used in the present invention may be in any form suitable for use in the methods described herein, such as, for example, a crude fermentation broth with or without cells or substantially pure polypeptides. The enzyme(s) may be a dry powder or granulate, a non-dusting granulate, a liquid, a stabilized liquid, or a protected enzyme(s). Granulates may be produced, e.g., as disclosed in U.S. Pat. Nos. 4,106,991 and 4,661,452, and may optionally be coated by process known in the art. Liquid enzyme preparations may, for instance, be stabilized by adding stabilizers such as a sugar, a sugar alcohol or another polyol, and/or lactic acid or another organic acid according to established process. Protected enzymes may be prepared according to the process disclosed in EP 238,216.

The optimum amounts of the enzymes and polypeptides having cellulolytic enhancing activity depend on several factors including, but not limited to, the mixture of component cellulolytic proteins, the cellulosic substrate, the concentration of cellulosic substrate, the pretreatment(s) of the cellulosic substrate, temperature, time, pH, and inclusion of fermenting organism(s) (e.g., yeast for Simultaneous Saccharification and Fermentation).

In a preferred aspect, an effective amount of cellulolytic protein(s) to cellulosic material is about 0.5 to about 50 mg, preferably at about 0.5 to about 40 mg, more preferably at about 0.5 to about 25 mg, more preferably at about 0.75 to about 20 mg, more preferably at about 0.75 to about 15 mg, even more preferably at about 0.5 to about 10 mg, and most preferably at about 2.5 to about 10 mg per 9 of cellulosic material.

In another preferred aspect, an effective amount of polypeptide(s) having cellulolytic enhancing activity to cellulosic material is about 0.01 to about 50.0 mg, preferably about 0.01 to about 40 mg, more preferably about 0.01 to about 30 mg, more preferably about 0.01 to about 20 mg, more preferably about 0.01 to about 10 mg, more preferably about 0.01 to about 5 mg, more preferably at about 0.025 to about 1.5 mg, more preferably at about 0.05 to about 1.25 mg, more preferably at about 0.075 to about 1.25 mg, more preferably at about 0.1 to about 1.25 mg, even more preferably at about 0.15 to about 1.25 mg, and most preferably at about 0.25 to about 1.0 mg per g of cellulosic material.

In another preferred aspect, an effective amount of polypeptide(s) having cellulolytic enhancing activity to cellulolytic protein(s) is about 0.005 to about 1.0 g, preferably at about 0.01 to about 1.0 g, more preferably at about 0.15 to about 0.75 g, more preferably at about 0.15 to about 0.5 g, more preferably at about 0.1 to about 0.5 g, even more preferably at about 0.1 to about 0.5 g, and most preferably at about 0.05 to about 0.2 g per g of cellulolytic protein(s).

Fermentation. The fermentable sugars obtained from the pretreated and hydrolyzed cellulosic material can be fermented by one or more fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from the cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous. Such methods include, but are not limited to, separate hydrolysis and fermentation (SHF); simultaneous saccharification and fermentation (SSF); simultaneous saccharification and cofermentation (SSCF); hybrid hydrolysis and fermentation (HHF); SHCF (separate hydrolysis and co-fermentation), HHCF (hybrid hydrolysis and fermentation), and direct microbial conversion (DMC).

Any suitable hydrolyzed cellulosic material can be used in the fermentation step in practicing the present invention. The material is generally selected based on the desired fermentation product, i.e., the substance to be obtained from the fermentation, and the process employed, as is well known in the art.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be C₆ and/or C₅ fermenting organisms, or a combination thereof. Both C₆ and C₅ fermenting organisms are well known in the art. Suitable fermenting microorganisms are able to ferment, i.e., convert, sugars, such as glucose, xylose, xylulose, arabinose, maltose, mannose, galactose, or oligosaccharides, directly or indirectly into the desired fermentation product. Some organisms also can convert soluble C6 and C5 oligomers.

Examples of bacterial and fungal fermenting organisms producing ethanol are described by Lin et al., 2006, Appl. Microbiol. Biotechnol. 69: 627-642

Examples of fermenting microorganisms that can ferment C6 sugars include bacterial and fungal organisms, such as yeast. Preferred yeast includes strains of the Saccharomyces spp., preferably Saccharomyces cerevisiae.

Examples of fermenting organisms that can ferment C5 sugars include bacterial and fungal organisms, such as yeast. Preferred C5 fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis, or Candida utilis.

Other fermenting organisms include strains of Zymomonas, such as Zymomonas mobilis; Hansenula, such as Hansenula anomala; Kluyveromyces, such as K. fragilis; Schizosaccharomyces, such as S. pombe; and E. coli, especially E. coli strains that have been genetically modified to improve the yield of ethanol.

In a preferred aspect, the yeast is a Saccharomyces spp. In a more preferred aspect, the yeast is Saccharomyces cerevisiae. In another more preferred aspect, the yeast is Saccharomyces distaticus. In another more preferred aspect, the yeast is Saccharomyces uvarum. In another preferred aspect, the yeast is a Kluyveromyces. In another more preferred aspect, the yeast is Kluyveromyces marxianus. In another more preferred aspect, the yeast is Kluyveromyces fragilis. In another preferred aspect, the yeast is a Candida. In another more preferred aspect, the yeast is Candida boidinii. In another more preferred aspect, the yeast is Candida brassicae. In another more preferred aspect, the yeast is Candida diddensii. In another more preferred aspect, the yeast is Candida pseudotropicalis. In another more preferred aspect, the yeast is Candida utilis. In another preferred aspect, the yeast is a Clavispora. In another more preferred aspect, the yeast is Clavispora lusitaniae. In another more preferred aspect, the yeast is Clavispora opuntiae. In another preferred aspect, the yeast is a Pachysolen. In another more preferred aspect, the yeast is Pachysolen tannophilus. In another preferred aspect, the yeast is a Pichia. In another more preferred aspect, the yeast is a Pichia stipitis. In another preferred aspect, the yeast is a Bretannomyces. In another more preferred aspect, the yeast is Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212).

Bacteria that can efficiently ferment hexose and pentose to ethanol include, for example, Zymomonas mobilis and Clostridium thermocellum (Philippidis, 1996, supra).

In a preferred aspect, the bacterium is a Zymomonas. In a more preferred aspect, the bacterium is Zymomonas mobilis. In another preferred aspect, the bacterium is a Clostridium. In another more preferred aspect, the bacterium is Clostridium thermocellum.

Commercially available yeast suitable for ethanol production includes, e.g., ETHANOL RED™ yeast (available from Fermentis/Lesaffre, USA), FALI™ (available from Fleischmann's Yeast, USA), SUPERSTART™ and THERMOSACC™ fresh yeast (available from Ethanol Technology, WI, USA), BIOFERM™ AFT and XR (available from NABC—North American Bioproducts Corporation, GA, USA), GERT STRAND™ (available from Gert Strand AB, Sweden), and FERMIOL™ (available from DSM Specialties).

In another aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (cofermentation) (Chen and Ho, 1993, Cloning and improving the expression of Pichia stipitis xylose reductase gene in Saccharomyces cerevisiae, Appl. Biochem. Biotechnol. 39-40: 135-147; Ho et al., 1998, Genetically engineered Saccharomyces yeast capable of effectively cofermenting glucose and xylose, Appl. Environ. Microbiol. 64: 1852-1859; Kotter and Ciriacy, 1993, Xylose fermentation by Saccharomyces cerevisiae, Appl. Microbiol. Biotechnol. 38: 776-783; Walfridsson et al., 1995, Xylose-metabolizing Saccharomyces cerevisiae strains overexpressing the TKL1 and TAL1 genes encoding the pentose phosphate pathway enzymes transketolase and transaldolase, Appl. Environ. Microbiol. 61: 4184-4190; Kuyper et al., 2004, Minimal metabolic engineering of Saccharomyces cerevisiae for efficient anaerobic xylose fermentation: a proof of principle, FEMS Yeast Research 4: 655-664; Beall et al., 1991, Parametric studies of ethanol production from xylose and other sugars by recombinant Escherichia coli, Biotech. Bioeng. 38: 296-303; Ingram et al., 1998, Metabolic engineering of bacteria for ethanol production, Biotechnol. Bioeng. 58: 204-214; Zhang et al., 1995, Metabolic engineering of a pentose metabolism pathway in ethanologenic Zymomonas mobilis, Science 267: 240-243; Deanda et al., 1996, Development of an arabinose-fermenting Zymomonas mobilis strain by metabolic pathway engineering, Appl. Environ. Microbiol. 62: 4465-4470).

In a preferred aspect, the genetically modified fermenting microorganism is Saccharomyces cerevisiae. In another preferred aspect, the genetically modified fermenting microorganism is Zymomonas mobilis. In another preferred aspect, the genetically modified fermenting microorganism is Escherichia coli. In another preferred aspect, the genetically modified fermenting microorganism is Klebsiella oxytoca.

It is well known in the art that the organisms described above can also be used to produce other substances, as described herein.

The fermenting microorganism is typically added to the degraded cellulosic material and the fermentation is performed for about 8 to about 96 hours, such as about 24 to about 60 hours. The temperature is typically between about 26° C. to about 60° C., in particular about 32° C. or 50° C., and at about pH 3 to about pH 8, such as around pH 4-5, 6, or 7.

In a preferred aspect, the yeast and/or another microorganism is applied to the degraded cellulosic material and the fermentation is performed for about 12 to about 96 hours, such as typically 24-60 hours. In a preferred aspect, the temperature is preferably between about 20° C. to about 60° C., more preferably about 25° C. to about 50° C., and most preferably about 32° C. to about 50° C., in particular about 32° C. or 50° C., and the pH is generally from about pH 3 to about pH 7, preferably around pH 4-7. However, some microorganisms, e.g., bacterial fermenting organisms, have higher fermentation temperature optima. Yeast or another microorganism is preferably applied in amounts of approximately 10⁵ to 10¹², more preferably from approximately 10⁷ to 10¹⁰, and especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the enzymatic processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation products: A fermentation product can be any substance derived from the fermentation. The fermentation product can be, without limitation, an alcohol (e.g., arabinitol, butanol, ethanol, glycerol, methanol, 1,3-propanediol, sorbitol, and xylitol); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); a ketone (e.g., acetone); an amino acid (e.g., aspartic acid, glutamic acid, glycine, lysine, serine, and threonine); and a gas (e.g., methane, hydrogen (H₂), carbon dioxide (CO₂), and carbon monoxide (CO)). The fermentation product can also be protein as a high value product.

In a preferred aspect, the fermentation product is an alcohol. It will be understood that the term “alcohol” encompasses a substance that contains one or more hydroxyl moieties. In a more preferred aspect, the alcohol is arabinitol. In another more preferred aspect, the alcohol is butanol. In another more preferred aspect, the alcohol is ethanol. In another more preferred aspect, the alcohol is glycerol. In another more preferred aspect, the alcohol is methanol. In another more preferred aspect, the alcohol is 1,3-propanediol. In another more preferred aspect, the alcohol is sorbitol. In another more preferred aspect, the alcohol is xylitol. See, for example, Gong, C. S., Cao, N.J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Silveira, M. M., and Jonas, R., 2002, The biotechnological production of sorbitol, Appl. Microbiol. Biotechnol. 59: 400-408; Nigam, P., and Singh, D., 1995, Processes for fermentative production of xylitol—a sugar substitute, Process Biochemistry 30 (2): 117-124; Ezeji, T. C., Qureshi, N. and Blaschek, H. P., 2003, Production of acetone, butanol and ethanol by Clostridium beijerinckii BA101 and in situ recovery by gas stripping, World Journal of Microbiology and Biotechnology 19 (6): 595-603.

In another preferred aspect, the fermentation product is an organic acid. In another more preferred aspect, the organic acid is acetic acid. In another more preferred aspect, the organic acid is acetonic acid. In another more preferred aspect, the organic acid is adipic acid. In another more preferred aspect, the organic acid is ascorbic acid. In another more preferred aspect, the organic acid is citric acid. In another more preferred aspect, the organic acid is 2,5-diketo-D-gluconic acid. In another more preferred aspect, the organic acid is formic acid. In another more preferred aspect, the organic acid is fumaric acid. In another more preferred aspect, the organic acid is glucaric acid. In another more preferred aspect, the organic acid is gluconic acid. In another more preferred aspect, the organic acid is glucuronic acid. In another more preferred aspect, the organic acid is glutaric acid. In another preferred aspect, the organic acid is 3-hydroxypropionic acid. In another more preferred aspect, the organic acid is itaconic acid. In another more preferred aspect, the organic acid is lactic acid. In another more preferred aspect, the organic acid is malic acid. In another more preferred aspect, the organic acid is malonic acid. In another more preferred aspect, the organic acid is oxalic acid. In another more preferred aspect, the organic acid is propionic acid. In another more preferred aspect, the organic acid is succinic acid. In another more preferred aspect, the organic acid is xylonic acid. See, for example, Chen, R., and Lee, Y. Y., 1997, Membrane-mediated extractive fermentation for lactic acid production from cellulosic biomass, Appl. Biochem. Biotechnol. 63-65: 435-448.

In another preferred aspect, the fermentation product is a ketone. It will be understood that the term “ketone” encompasses a substance that contains one or more ketone moieties. In another more preferred aspect, the ketone is acetone. See, for example, Qureshi and Blaschek, 2003, supra.

In another preferred aspect, the fermentation product is an amino acid. In another more preferred aspect, the organic acid is aspartic acid. In another more preferred aspect, the amino acid is glutamic acid. In another more preferred aspect, the amino acid is glycine. In another more preferred aspect, the amino acid is lysine. In another more preferred aspect, the amino acid is serine. In another more preferred aspect, the amino acid is threonine. See, for example, Richard, A., and Margaritis, A., 2004, Empirical modeling of batch fermentation kinetics for poly(glutamic acid) production and other microbial biopolymers, Biotechnology and Bioengineering 87 (4): 501-515.

In another preferred aspect, the fermentation product is a gas. In another more preferred aspect, the gas is methane. In another more preferred aspect, the gas is H₂. In another more preferred aspect, the gas is CO₂. In another more preferred aspect, the gas is CO. See, for example, Kataoka, N., A. Miya, and K. Kiriyama, 1997, Studies on hydrogen production by continuous culture system of hydrogen-producing anaerobic bacteria, Water Science and Technology 36 (67): 41-47; and Gunaseelan V. N. in Biomass and Bioenergy, Vol. 13 (1-2), pp. 83-114, 1997, Anaerobic digestion of biomass for methane production: A review.

Recovery. The fermentation product(s) can be optionally recovered from the fermentation medium using any method known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### Tannases

In the methods of the present invention, any tannase may be used. The tannase can be obtained from any source, especially microorganisms of any genus. For purposes of the present invention, the term “obtained from” is used as defined herein. In a preferred aspect, the tannase obtained from a given source is secreted extracellularly.

The tannase may be a bacterial tannase. For example, the tannase may be a gram positive bacterial tannase such as a Bacillus, Corynebacterium, Streptococcus, Streptomyces, Staphylococcus, Enterococcus, Lactobacillus, Lactococcus, Clostridium, Geobacillus, or Oceanobacillus tannase, or a Gram negative bacterial tannase such as an E. coli, Pseudomonas, Salmonella, Campylobacter, Helicobacter, Flavobacterium, Fusobacterium, Ilyobacter, Neisseria, or Ureaplasma tannase.

In a preferred aspect, the tannase is a Bacillus alkalophilus, Bacillus amyloliquefaciens, Bacillus brevis, Bacillus cereus, Bacillus circulans, Bacillus clausii, Bacillus coagulans, Bacillus firmus, Bacillus lautus, Bacillus lentus, Bacillus licheniformis, Bacillus megaterium, Bacillus polymyxa, Bacillus pumilus, Bacillus stearothermophilus, Bacillus subtilis, Bacillus thuringiensis, Lactobacillus plantarum, Streptococcus equisimilis, Streptococcus pyogenes, Streptococcus uberis, or Streptococcus equi subsp. Zooepidemicus tannase.

In another preferred aspect, the tannase is a Streptomyces achromogenes, Streptomyces avermitilis, Streptomyces coelicolor, Streptomyces griseus, or Streptomyces lividans tannase.

The tannase may also be a fungal tannase, and more preferably a yeast tannase such as a Candida, Kluyveromyces, Pichia, Saccharomyces, Schizosaccharomyces, or Yarrowia tannase; or more preferably a filamentous fungal tannase such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Rhizopus, Schizophyllum, Scytalidium, Talaromyces, Thermoascus, Thielavia, Tolypocladium, Trichoderma, Trichophaea, Verticillium, Volvariella, or Xylaria tannase.

In a preferred aspect, the tannase is a Saccharomyces carlsbergensis, Saccharomyces cerevisiae, Saccharomyces diastaticus, Saccharomyces douglasii, Saccharomyces kluyven, Saccharomyces norbensis, or Saccharomyces ovifommis tannase.

In another preferred aspect, the tannase is an Acremonium cellulolyticus, Aspergillus aculeatus, Aspergillus awamori, Aspergillus fischeri, Aspergillus flavus, Aspergillus foetidus, Aspergillus fumigatus, Aspergillus japonicus, Aspergillus nidulans, Aspergillus niger (TrEMBL Accession Nos. A2Q818, A2QAH7, A2QBC9, A2QBK3, A2QH22, A2QIR3, A2QS33, A2QT57, A2QV40, A2QV44, A2QVF5, A2QW25, A2R0Z6, A2R274, and A2R9CO), Aspergillus oryzae (Swiss-Prot Accession number P78581), Aspergillus usamii, Aspergillus ustus, Aspergillus versicolor, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusanum heterosporum, Fusarium negundi, Fusarium oxysporum, Fusarium reticulatum, Fusarium roseum, Fusarium sambucinum, Fusarium sarcochroum, Fusarium solani, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Paecilomyces variotii, Penicillium charlesii, Penicillium chrysogenum, Penicillium expansum, Penicillium funiculosum, Penicillium javanicum, Penicillium notatum, Penicillium oxaicum, Penicillium purpurogenum, Penicillium restrictum, Penicillium variabile, Phanerochaete chrysosporium, Rhizopus oryzae, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthernophila, Thielavia terrestris, Trichoderrna harzianum, Trichoderma koningii, Trichoderma longibrachiatum, Trichoderma reesei, or Trichoderma viride tannase.

In another preferred aspect, the tannase comprises or consists of SEQ ID NO: 2, SEQ ID NO: 4, SEQ ID NO: 6, SEQ ID NO: 8, or SEQ ID NO: 10, or a fragment thereof that has tannase activity. In another preferred aspect, the tannase is the mature tannase of SEQ ID NO: 2, SEQ ID NO: 4, SEQ ID NO: 6, SEQ ID NO: 8, or SEQ ID NO: 10. In another preferred aspect, the tannase is encoded by SEQ ID NO: 1, SEQ ID NO: 3, SEQ ID NO: 5, SEQ ID NO: 7, or SEQ ID NO: 9, or a subsequence thereof that encodes a polypeptide fragment that has tannase activity. In another preferred aspect, the tannase is encoded by the mature polypeptide coding sequence of SEQ ID NO: 1, SEQ ID NO: 3, SEQ ID NO: 5, SEQ ID NO: 7, or SEQ ID NO: 9.

In a more preferred aspect, the tannase is an Aspergillus oryzae tannase. In a most preferred aspect, the tannase comprises or consists of SEQ ID NO: 2, or a fragment thereof that has tannase activity. In another most preferred aspect, the tannase comprises or consists of the mature tannase of SEQ ID NO: 2, or a fragment thereof that has tannase activity.

It will be understood that for the aforementioned species the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen und Zellkulturen GmbH (DSM), Centraalbureau Voor Schimmelcultures (CBS), and Agricultural Research Service Patent Culture Collection, Northern Regional Research Center (NRRL).

Furthermore, such tannases may be identified and obtained from other sources including microorganisms isolated from nature (e.g., soil, composts, water, etc.) using the above-mentioned probes. Techniques for isolating microorganisms from natural habitats are well known in the art. The polynucleotide may then be obtained by similarly screening a genomic or cDNA library of such a microorganism. Once a polynucleotide sequence encoding a tannase has been detected with the probe(s), the polynucleotide can be isolated or cloned by utilizing techniques that are well known to those of ordinary skill in the art (see, e.g., Sambrook et al., 1989, supra).

Tannases also include fused polypeptides or cleavable fusion polypeptides in which another polypeptide is fused at the N-terminus or the C-terminus of the tannase or fragment thereof. A fused polypeptide is produced by fusing a nucleotide sequence (or a portion thereof) encoding another polypeptide to a nucleotide sequence (or a portion thereof) of the present invention. Techniques for producing fusion polypeptides are known in the art, and include ligating the coding sequences encoding the polypeptides so that they are in frame and that expression of the fused polypeptide is under control of the same promoter(s) and terminator.

A fusion polypeptide can further comprise a cleavage site. Upon secretion of the fusion protein, the site is cleaved releasing the tannase from the fusion protein. Examples of cleavage sites include, but are not limited to, a Kex2 site that encodes the dipeptide Lys-Arg (Martin et al., 2003, J. Ind. Microbiol. Biotechnol. 3: 568-76; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381), an Ile-(Glu or Asp)-Gly-Arg site, which is cleaved by a Factor Xa protease after the arginine residue (Eaton et al., 1986, Biochem. 25: 505-512); a Asp-Asp-Asp-Asp-Lys site, which is cleaved by an enterokinase after the lysine (Collins-Racie et al., 1995, Biotechnology 13: 982-987); a His-Tyr-Glu site or His-Tyr-Asp site, which is cleaved by Genenase I (Carter et al., 1989, Proteins: Structure, Function, and Genetics 6: 240-248); a Leu-Val-Pro-Arg-Gly-Ser site, which is cleaved by thrombin after the Arg (Stevens, 2003, Drug Discovery World 4: 35-48); a Glu-Asn-Leu-Tyr-Phe-Gln-Gly site, which is cleaved by TEV protease after the Gln (Stevens, 2003, supra); and a Leu-Glu-Val-Leu-Phe-Gln-Gly-Pro site, which is cleaved by a genetically engineered form of human rhinovirus 3C protease after the Gln (Stevens, 2003, supra).

Examples of other tannases useful in the present invention are listed in Table 1.

TABLE 1

| AUTHORS | TITLE | JOURNAL | ORGANISM |
|---|---|---|---|
| Rajakumar, G. S.; | Isolation, purification, and some properties of | Appl. Environ. | Penicillium chrysogenum |
| Nandy, S. C. | Penicillium chrysogenum tannase | Microbiol. 46: 525-527 (1983) | |
| Deschamps, A. M.; | Production of tannase and degradation of chestnut tannin by | J. Ferment. Technol. | Corynebacterium sp., |
| Otuk, G.; | bacteria | 61: 55-59 (1983) | Klebsiella pneumoniae, |
| Lebeault, J. M. | | | Bacillus pumilus, Bacillus |
| | | | polymyxa |
| Aoki, K.; Shinke, R.; | Chemical composition and molecular weight of yeast tannase | Agric. Biol. Chem. 40: | Candida sp. |
| Nishira, H. | | 297-302 (1976) | |
| Aoki, K.; Shinke, R.; | Purification and some properties of yeast tannase | Agric. Biol. Chem. 40: | Candida sp. |
| Nishira, H. | | 79-85 (1976) | |
| libuchi, S.; | Hydrolizing pathway, substrate specificity and | Agric. Biol. Chem. 36: | Aspergillus oryzae |
| Minoda, Y.; | inhibition of tannin acyl hydrolase of Asp. oryzae No. 7 | 1553-1562 (1972) | |
| Yamada, K. | | | |
| Yamada et al. | Studies on fungal tannase. Part I. Formation, | Agric. Biol. Chem. 32: | Aspergillus niger, Penicillium |
| | purification and catalytic properties of tannase of | 1070-1078 (1968) | notatum, Aspergillus flavus, |
| | Aspergillus flavus | | Aspergillus oryzae, |
| | | | Aspergillus sojae, Penicillium |
| | | | oxalicum, Aspergillus |
| | | | awamori, Penicillium |
| | | | expansum, Aspergillus ustus, |
| | | | Aspergillus usamii, |
| | | | Penicillium javanicum |
| Adachi et al. | Studies on fungal tannase. Part II. Physicochemical | Agric. Biol. Chem. 32: | Aspergillus flavus |
| | properties of tannase of Aspergillus flavus | 1079-1085 (1968) | |
| libuchi et al. | Studies on tannin acyl hydrolase of microorganisms. | Agric. Biol. Chem. 32: | Aspergillus oryzae |
| | Part III. Purification of the enzyme and some | 803-809 (1968) | |
| | proporties of it | | |
| Yamada et al. | Tannase (tannin acyl hydrolase), a typical serine | Agric. Biol. Chem. 32: | Aspergillus flavus |
| | esterase | 257-258 (1968) | |
| Lekha and | Comparative titres, location and properties of tannin | Proc. Biochem. 29: | Aspergillus niger |
| Lonsane | acyl hydrolase produced by Aspergillus niger PKL | 497-503 (1994) | |
| | 104 in solid-state, liquid surface and submerged | | |
| | fermentations | | |
| Niehaus and | A gallotannin degrading esterase from leaves of pedunculate oak | Phytochemistry 45: | Quercus robur |
| Gross | | 1555-1560 (1997) | |
| Beverini and | Identification, purification and physicochemical | Sci. Aliments 10: 807-816 | Aspergillus oryzae |
| Metche | properties of tannase of Aspergillus orizae | (1990) | |
| Skene and | Characterization of tannin acylhydrolase activity in | Anaerobe 1: 321-327 | Selenomonas ruminantium |
| Brooker | the ruminal bacterium Selenomonas ruminantium | (1995) | |
| Barthomeuf et | Production, purification and characterization of a | J. Ferment. Bioeng. 77: | Aspergillus niger |
| al. | tannase from Aspergillus niger LCF 8 | 320-323 (1994) | |
| Hatamoto et al. | Cloning and sequencing of the gene encoding | Gene 175: 215-221 | Aspergillus oryzae |
| | tannase and a structural study of the tannase subunit | (1996) | |
| | from Aspergillus oryzae | | |
| Saxena and | Statistical optimization of tannase production from | Biotechnol. Appl. | Penicillium variabile |
| Saxena | Penicillium variable using fruits (chebulic myrobalan) | Biochem. 39: 99-106 | |
| | of Terminalia chebula | (2004) | |
| Ayed, L.; Hamdi, M. | Culture conditions of tannase production by | Biotechnol. Lett. 24: | Lactobacillus plantarum |
| | Lactobacillus plantarum | 1763-1765 (2002) | |
| Aguilar and; | Review: sources, properties, applications and | Food Sci. Technol. Int. | Phaseolus vulgaris, Bos |
| Gutierrez- | potential uses of tannin acyl hydrolase | 7: 373-382 (2001) | taurus, Aspergillus niger, |
| Sanchez | | | Aspergillus fischeri, |
| | | | Aspergillus flavus, |
| | | | Aspergillus oryzae, Fusarium |
| | | | solani, Aspergillus japonicus, |
| | | | Trichoderma viride, Rhizopus |
| | | | oryzae, Cryphonectria |
| | | | parasitica |
| Mondal and Pati | Studies on the extracellular tannase from newly | J. Basic Microbiol. 40: | Bacillus licheniformis |
| | isolated Bacillus licheniformis KBR 6 | 223-232 (2000) | |
| Banerjee et al. | Production and characterization of extracellular and | J. Basic Microbiol. 41: | Aspergillus aculeatus |
| | intracellular tannase from newly isolated Aspergillus | 313-318 (2001) | |
| | aculeatus DBF 9 | | |
| Bhardwaj et al. | Purification and characterization of tannin acyl | J. Basic Microbiol. 43: | Aspergillus niger |
| | hydrolase from Aspergillus niger MTCC 2425 | 449-461 (2003) | |
| Mukherjee and | Biosynthesis of tannase and gallic acid from tannin | J. Basic Microbiol. 44: | Aspergillus foetidus, |
| Banerjee | rich substrates by Rhizopus oryzae and Aspergillus | 42-48 (2004) | Rhizopus oryzae |
| | foetidus | | |
| Mondal et al. | Production and characterization of tannase from | J. Gen. Appl. Microbiol. | Bacillus cereus |
| | Bacillus cereus KBR9 | 47: 263-267 (2001) | |
| Ramirez- | A novel tannase from Aspergillus niger with beta- | Microbiology 149: | Aspergillus niger |
| Coronel et al. | glucosidase activity | 2941-2946 (2003) | |
| Kar et al. | Effect of additives on the behavioural properties of | Proc. Biochem. 38: | Rhizopus oryzae |
| | tannin acyl hydrolase | 1285-1293 (2003) | |
| Mahendran et | Purification and characterization of tannase from | Appl. Microbiol. | Paecilomyces variotii |
| al. | Paecilomyces variotii: hydrolysis of tannic acid using | Biotechnol. 70: 444-450 | |
| | immobilized tannase | (2006) | |
| Sabu et al. | Purification and characterization of tannin acyl | Food Technol. | Aspergillus niger |
| | hydrolase from Aspergillus niger ATCC 16620 | Biotechnol. 43: 133-138 | |
| | | (2005 | |
| Vaquero et al. | Tannase activity by lactic acid bacteria isolated from | Int. J. Food Microbiol. | Lactobacillus plantarum |
| | grape must and wine | 96: 199-204 (2004) | |
| Rana et al. | Effect of fermentation system on the production and | J. Gen. Appl. Microbiol. | Aspergillus niger |
| | properties of tannase of Aspergillus niger van | 51: 203-212 (2005) | |
| | Tieghem MTCC 2425 | | |
| Yu et al.. | Enzymatic synthesis of gallic acid esters using | J. Mol. Catal. B 30: 69-73 | Aspergillus niger |
| | microencapsulated tannase: effect of organic | (2004) | |
| | solvents and enzyme specificity | | |
| Batra and | Potential tannase producers from the genera | Proc. Biochem. 40: | Aspergillus flavus |
| Saxena | Aspergillus and Penicillium | 1553-1557 (2005) | |
| Huang et al. | Biosynthesis of valonia tannin hydrolase and | Process Biochem. 40: | Aspergillus sp. |
| | hydrolysis of valonia tannin to ellagic acid by | 1245-1249 (2004) | |
| | Aspergillus SHL 6 | | |
| Batra and | Potential tannase producers from the genera | Process Biochem. 40: | Aspergillus fumigatus, |
| Saxena | Aspergillus and Penicillium | 1553-1557 (2005) | Aspergillus versicolor, |
| | | | Penicillium charlesi, |
| | | | Penicillium restrictum |
| Mahapatra et al. | Purification, characterization and some studies on | Process Biochem. 40: | Aspergillus awamori |
| | secondary structure of tannase from Aspergillus | 3251-3254 (2005) | |
| | awamori Nakazawa | | |
| Sabu et al. | Tannase production by Lactobacillus sp. ASR-S1 | Process Biochem. 41: | Lactobacillus sp. |
| | under solid-state fermentation | 575-580 (2006) | |
| Zhong et al. | Secretion, purification, and characterization of a | Protein Expr. Purif. 36: | Aspergillus oryzae |
| | recombinant Aspergillus oryzae tannase in Pichia | 165-169 (2004) | |
| | pastoris | | |
| Aissam et al. | Production of tannase by Aspergillus niger HA37 | World J. Microbiol. | Aspergillus niger |
| | growing on tannic acid and Olive Mill Waste Waters | Biotechnol. 21: 609-614 | |
| | | (2005) | |

Examples of commercial tannase preparations suitable for use in the present invention include, for example, an Aspergillus oryzae tannase (available from Novozymes A/S), and tannases from Kikkoman Corp of Tokyo, Japan, and Juelich Enzyme Products GmbH of Wiesbaden, Germany.

### Cellulolytic Enzyme Compositions

In the methods of the present invention, the cellulolytic enzyme composition may comprise any protein involved in the processing of a cellulosic material, e.g., lignocellulose, to fermentable sugars, e.g., glucose.

For cellulose degradation, at least three categories of enzymes are important for converting cellulose into fermentable sugars: endo-glucanases (EC 3.2.1.4) that hydrolyze the cellulose chains at random; cellobiohydrolases (EC 3.2.1.91) that cleave cellobiosyl units from the cellulose chain ends, and beta-glucosidases (EC 3.2.1.21) that convert cellobiose and soluble cellodextrins into glucose.

The cellulolytic enzyme composition may be a monocomponent preparation, e.g., an endoglucanase, a multicomponent preparation, e.g., endoglucanase, cellobiohydrolase, beta-glucosidase, or a combination of multicomponent and monocomponent protein preparations. The cellulolytic proteins may have activity, i.e., hydrolyze cellulose, either in the acid, neutral, or alkaline pH range.

A polypeptide having cellulolytic enzyme activity may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having cellulolytic enzyme activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having cellulolytic enzyme activity.

In a preferred aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having cellulolytic enzyme activity.

In another preferred aspect, the polypeptide is a Streptococcus equisimilis, Streptococcus pyogenes, Streptococcus uberis, or Streptococcus equi subsp. Zooepidemicus polypeptide having cellulolytic enzyme activity.

In another preferred aspect, the polypeptide is a Streptomyces achromogenes, Streptomyces avermitilis, Streptomyces coelicolor, Streptomyces griseus, or Streptomyces lividans polypeptide having cellulolytic enzyme activity.

The polypeptide having cellulolytic enzyme activity may also be a fungal polypeptide, and more preferably a yeast polypeptide such as a Candida, Kluyveromyces, Pichia, Saccharomyces, Schizosaccharomyces, or Yarrowia polypeptide having cellulolytic enzyme activity; or more preferably a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thernoascus, Thielavia, Tolypocladium, Trichoderma, Trichophaea, Verticillium, Volvariella, or Xylaria polypeptide having cellulolytic enzyme activity.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having cellulolytic enzyme activity.

In another preferred aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusanum venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having cellulolytic enzyme activity.

Chemically modified or protein engineered mutants of cellulolytic proteins may also be used.

One or more components of the cellulolytic enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic proteins may also be prepared by purifying such a protein from a fermentation broth.

The cellulolytic proteins used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and cellulolytic protein production are known in the art (see, e.g., Bailey, J. E., and Ollis, D. F., Biochemical Engineering Fundamentals, McGraw-Hill Book Company, NY, 1986).

The fermentation can be any method of cultivation of a cell resulting in the expression or isolation of a cellulolytic protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the cellulolytic protein to be expressed or isolated. The resulting cellulolytic proteins produced by the methods described above may be recovered from the fermentation medium and purified by conventional procedures as described herein.

Examples of commercial cellulolytic enzyme preparations suitable for use in the present invention include, for example, CELLUCLAST™ (available from Novozymes A/S) and NOVOZYM™ 188 (available from Novozymes A/S). Other commercially available preparations comprising cellulase that may be used include CELLUZYME™, CEREFLO™ and ULTRAFLO™ (Novozymes A/S), LAMINEX™ and SPEZYME™ CP (Genencor Int.), ROHAMENT™ 7069 W (Röhm GmbH), and FIBREZYME® LDI, FIBREZYME® LBR, or VISCOSTAR® 150L (Dyadic International, Inc., Jupiter, Fla., USA). The cellulase enzymes are added in amounts effective from about 0.001% to about 5.0% wt. of solids, more preferably from about 0.025% to about 4.0% wt. of solids, and most preferably from about 0.005% to about 2.0% wt. of solids.

Examples of bacterial endoglucanases that can be used in the methods of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the methods of the present invention, include, but are not limited to, a Trichoderma reesei endoglucanase I (Penttila et al., 1986, Gene 45: 253-263; GENBANK™ accession no. M15665); Trichoderma reesei endoglucanase II (Saloheimo, et al., 1988, Gene 63:11-22; GENBANK™ accession no. M19373); Trichoderma reesei endoglucanase III (Okada et al., 1988, Appl. Environ. Microbiol. 64: 555-563; GENBANK™ accession no. AB003694); Trichoderma reesei endoglucanase IV (Saloheimo et al., 1997, Eur. J. Biochem. 249: 584-591; GENBANK™ accession no. Y11113); and Trichoderma reesei endoglucanase V (Saloheimo et al., 1994, Molecular Microbiology 13: 219-228; GENBANK™ accession no. Z33381); Aspergillus aculeatus endoglucanase (Ooi et al., 1990, Nucleic Acids Research 18: 5884); Aspergillus kawachii endoglucanase (Sakamoto et al., 1995, Current Genetics 27: 435-439); Erwinia carotovara endoglucanase (Saarilahti et al., 1990, Gene 90: 9-14); Fusarium oxysporum endoglucanase (GENBANK™ accession no. L29381); Humicola grisea var. thermoidea endoglucanase (GENBANK™ accession no. AB003107); Melanocarpus albomyces endoglucanase (GENBANK™ accession no. MAL515703); Neurospora crassa endoglucanase (GENBANK™ accession no. XM_{—}324477); Humicola insolens endoglucanase V (SEQ ID NO: 12); Myceliophthora thermophila CBS 117.65 endoglucanase (SEQ ID NO: 14); basidiomycete CBS 495.95 endoglucanase (SEQ ID NO: 16); basidiomycete CBS 494.95 endoglucanase (SEQ ID NO: 18); Thielavia terrestris NRRL 8126 CEL6B endoglucanase (SEQ ID NO: 20); Thielavia terrestris NRRL 8126 CEL6C. endoglucanase (SEQ ID NO: 22); Thielavia terresttis NRRL 8126 CEL7C endoglucanase (SEQ ID NO: 24); Thielavia terrestris NRRL 8126 CEL7E endoglucanase (SEQ ID NO: 26); Thielavia terrestris NRRL 8126 CEL7F endoglucanase (SEQ ID NO: 28); Cladorrhinum foecundissimum ATCC 62373 CEL7A endoglucanase (SEQ ID NO: 30); and Trichoderma reesei strain No. VTT-D-80133 endoglucanase (SEQ ID NO: 32; GENBANK™ accession no. M15665). The endoglucanases of SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, and SEQ ID NO: 32 described above are encoded by the mature polypeptide coding sequence of SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, and SEQ ID NO: 31, respectively.

Examples of cellobiohydrolases useful in the methods of the present invention include, but are not limited to, Trichoderma reesei cellobiohydrolase I (SEQ ID NO: 34); Trichoderma reesei cellobiohydrolase II (SEQ ID NO: 36); Humicola insolens cellobiohydrolase I (SEQ ID NO: 38), Myceliophthora thermophila cellobiohydrolase II (SEQ ID NO: 40), Thielavia terrestris cellobiohydrolase II (CEL6A) (SEQ ID NO: 42), Chaetomium thermophilum cellobiohydrolase I (SEQ ID NO: 44), and Chaetomium thermophilum cellobiohydrolase II (SEQ ID NO: 46). The cellobiohydrolases of SEQ ID NO: 34, SEQ ID NO: 36, SEQ ID NO: 38, SEQ ID NO: 40, SEQ ID NO: 42, SEQ ID NO: 44, and SEQ ID NO: 46 described above are encoded by the mature polypeptide coding sequence of SEQ ID NO: 33, SEQ ID NO: 35, SEQ ID NO: 37, SEQ ID NO: 39, SEQ ID NO: 41, SEQ ID NO: 43, and SEQ ID NO: 45, respectively.

Examples of beta-glucosidases useful in the methods of the present invention include, but are not limited to, Aspergillus oryzae beta-glucosidase (SEQ ID NO: 48); Aspergillus fumigatus beta-glucosidase (SEQ ID NO: 50); Penicillium brasilianum IBT 20888 beta-glucosidase (SEQ ID NO: 52); Aspergillus niger beta-glucosidase (SEQ ID NO: 54); and Aspergillus aculeatus beta-glucosidase (SEQ ID NO: 56). The beta-glucosidases of SEQ ID NO: 48, SEQ ID NO: 50, SEQ ID NO: 52, SEQ ID NO: 54, and SEQ ID NO: 56 described above are encoded by the mature polypeptide coding sequence of SEQ ID NO: 47, SEQ ID NO: 49, SEQ ID NO: 51, SEQ ID NO: 53, and SEQ ID NO: 55, respectively.

The Aspergillus oryzae polypeptide having beta-glucosidase activity can be obtained according to WO 2002/095014. The Aspergillus fumigatus polypeptide having beta-glucosidase activity can be obtained according to WO 2005/047499. The Penicillium brasilianum polypeptide having beta-glucosidase activity can be obtained according to WO 2007/019442. The Aspergillus niger polypeptide having beta-glucosidase activity can be obtained according to Dan et al., 2000, J. Biol. Chem. 275: 4973-4980. The Aspergillus aculeatus polypeptide having beta-glucosidase activity can be obtained according to Kawaguchi et al., 1996, Gene 173: 287-288.

Other endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695696.

In another preferred aspect, the beta-glucosidase is the Aspergillus oryzae beta-glucosidase variant BG fusion protein of SEQ ID NO: 58 or the Aspergillus oryzae beta-glucosidase fusion protein of SEQ ID NO: 60. In another preferred aspect, the Aspergillus oryzae beta-glucosidase variant BG fusion protein is encoded by the polynucleotide of SEQ ID NO: 57 or the Aspergillus oryzae beta-glucosidase fusion protein is encoded by the polynucleotide of SEQ ID NO: 59.

The cellulolytic enzyme composition may further comprise a polypeptide(s) having cellulolytic enhancing activity, comprising the following motifs:
[ILMV]-P—X(4,5)-G-X-Y-[ILMV]-X-R-X-[EQ]-X(4)-[HNQ] and [FW]-[TF]-K-[AIV],
   wherein X is any amino acid, X(4,5) is any amino acid at 4 or 5 contiguous positions, and X(4) is any amino acid at 4 contiguous positions.

The isolated polypeptide comprising the above-noted motifs may further comprise:
H-X(1,2)-G-P-X(3)-[YW]-[AILMV],
[EQ]-X-Y-X(2)-C-X-[EHQN]-[FILV]-X-[ILV], or
H-X(1,2)-G-P-X(3)-[YW]-[AILMV] and [EQ]-X-Y-X(2)-C-X-[EHQN]-[FILV]-X-[ILV],
   wherein X is any amino acid, X(1,2) is any amino acid at 1 position or 2 contiguous positions, X(3) is any amino acid at 3 contiguous positions, and X(2) is any amino acid at 2 contiguous positions. In the above motifs, the accepted IUPAC single letter amino acid abbreviation is employed.

In a preferred aspect, the isolated polypeptide having cellulolytic enhancing activity further comprises H—X(1,2)-G-P-X(3)-[YW]-[AILMV]. In another preferred aspect, the isolated polypeptide having cellulolytic enhancing activity further comprises [EQ]-X-Y-X(2)-C-X-[EHQN]-[FILV]-X-[ILV]. In another preferred aspect, the isolated polypeptide having cellulolytic enhancing activity further comprises H—X(1,2)-G-P-X(3)-[YW]-[AILMV] and [EQ]-X-Y-X(2)-C-X-[EHQN]-[FILV]-X-[ILV].

Examples of isolated polypeptides having cellulolytic enhancing activity include Thielavia terrestris polypeptides having cellulolytic enhancing activity (the mature polypeptide of SEQ ID NO: 62, SEQ ID NO: 64, SEQ ID NO: 66, SEQ ID NO: 68, SEQ ID NO: 70, or SEQ ID NO: 72); Thermoascus auranticus (the mature polypeptide of SEQ ID NO: 74), or Trichoderma reesei (the mature polypeptide of SEQ ID NO: 76). The polypeptides having cellulolytic enhancing activity of SEQ ID NO: 62, SEQ ID NO: 64, SEQ ID NO: 66, SEQ ID NO: 68, SEQ ID NO: 70, SEQ ID NO: 72, and SEQ ID NO: 74, described above, are encoded by the mature polypeptide coding sequence of SEQ ID NO: 61, SEQ ID NO: 63, SEQ ID NO: 65, SEQ ID NO: 67, SEQ ID NO: 69, SEQ ID NO: 71, SEQ ID NO: 73, and SEQ ID NO: 75, respectively.

For further details on polypeptides having cellulolytic enhancing activity and polynucleotides thereof, see WO 2005/074647, WO 2005/074656, and U.S. Published Application Serial No. 2007/0077630, which are incorporated herein by reference.

The cellulolytic enzyme composition may further comprise one or more enzymes selected from the group consisting of a hemicellulase, esterase, protease, laccase, peroxidase, or a mixture thereof.

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, endo-galactanase, mannases, endo or exo arabinases, exo-galactanses, xylosidases, and combinations thereof. Preferably, the hemicellulase has the ability to hydrolyze hemicellulose under acidic conditions of below pH 7, preferably pH 3-7. An example of hemicellulase suitable for use in the present invention includes VISCOZYME™ (available from Novozymes A/S, Denmark).

In one aspect, the hemicellulase is a xylanase. The xylanase may be of microbial origin, such as fungal origin (e.g., Trichoderma, Meripilus, Humicola, Aspergillus, Fusarium) or bacterial origin (e.g., Bacillus). In a preferred aspect, the xylanase is obtained from a filamentous fungus, preferably from a strain of Aspergillus, such as Aspergillus aculeatus; or a strain of Humicola, such as Humicola lanuginosa. The xylanase is preferably an endo-1,4-beta-xylanase, more preferably an endo-1,4-beta-xylanase of GH10 or GH11. Examples of commercial xylanases include SHEARZYME™ and BIOFEED WHEAT™ (Novozymes A/S, Denmark).

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amount of 0.005-0.5 g/kg DM substrate, and most preferably from 0.05-0.10 g/kg DM substrate.

### Nucleic Acid Constructs

An isolated polynucleotide encoding a polypeptide having enzyme activity, e.g., tannase, or cellulolytic enhancing activity may be manipulated in a variety of ways to provide for expression of the polypeptide by constructing a nucleic acid construct comprising an isolated polynucleotide encoding the polypeptide operably linked to one or more control sequences that direct the expression of the coding sequence in a suitable host cell under conditions compatible with the control sequences. Manipulation of the polynucleotide's sequence prior to its insertion into a vector may be desirable or necessary depending on the expression vector. The techniques for modifying polynucleotide sequences utilizing recombinant DNA methods are well known in the art.

The control sequence may be an appropriate promoter sequence, a nucleotide sequence that is recognized by a host cell for expression of a polynucleotide encoding such a polypeptide. The promoter sequence contains transcriptional control sequences that mediate the expression of the polypeptide. The promoter may be any nucleotide sequence that shows transcriptional activity in the host cell of choice including mutant, truncated, and hybrid promoters, and may be obtained from genes encoding extracellular or intracellular polypeptides either homologous or heterologous to the host cell.

Examples of suitable promoters for directing the transcription of the nucleic acid constructs, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (VIIIa-Kamaroff et al., 1978, Proceedings of the National Academy of Sciences USA 75: 3727-3731), as well as the tac promoter (DeBoer et al., 1983, Proceedings of the National Academy of Sciences USA 80: 21-25). Further promoters are described in “Useful proteins from recombinant bacteria” in Scientific American, 1980, 242: 74-94; and in Sambrook et al., 1989, supra.

Examples of suitable promoters for directing the transcription of the nucleic acid constructs' in a filamentous fungal host cell are promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, Fusarium venenatum amyloglucosidase (WO 00/56900), Fusarium venenatum Daria (WO 00/56900), Fusarium venenatum Quinn (WO 00/56900), Fusarium oxysporum trypsin-like protease (WO 96/00787), Trichoderma reesei betaglucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase 1, Trichoderma reesei xylanase II, Trichoderma reesei beta-xylosidase, as well as the NA2-tpi promoter (a hybrid of the promoters from the genes for Aspergillus niger neutral alpha-amylase and Aspergillus oryzae triose phosphate isomerase); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8: 423-488.

The control sequence may also be a suitable transcription terminator sequence, a sequence recognized by a host cell to terminate transcription. The terminator sequence is operably linked to the 3′ terminus of the nucleotide sequence encoding the polypeptide. Any terminator that is functional in the host cell of choice may be used in the present invention.

Preferred terminators for filamentous fungal host cells are obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be a suitable leader sequence, a nontranslated region of an mRNA that is important for translation by the host cell. The leader sequence is operably linked to the 5′ terminus of the nucleotide sequence encoding the polypeptide. Any leader sequence that is functional in the host cell of choice may be used in the present invention.

Preferred leaders for filamentous fungal host cells are obtained from the genes for Aspergillus oryzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′ terminus of the nucleotide sequence and, when transcribed, is recognized by the host cell as a signal to add polyadenosine residues to transcribed mRNA. Any polyadenylation sequence that is functional in the host cell of choice may be used in the present invention.

Preferred polyadenylation sequences for filamentous fungal host cells are obtained from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase.

Useful polyadenylation sequences for yeast host cells are described by Guo and Sherman, 1995, Molecular Cellular Biology 15: 59835990.

The control sequence may also be a signal peptide coding sequence that codes for an amino acid sequence linked to the amino terminus of a polypeptide and directs the encoded polypeptide into the cell's secretory pathway. The 5′ end of the coding sequence of the nucleotide sequence may inherently contain a signal peptide coding sequence naturally linked in translation reading frame with the segment of the coding region that encodes the secreted polypeptide. Alternatively, the 5′ end of the coding sequence may contain a signal peptide coding sequence that is foreign to the coding sequence. The foreign signal peptide coding sequence may be required where the coding sequence does not naturally contain a signal peptide coding sequence. Alternatively, the foreign signal peptide coding sequence may simply replace the natural signal peptide coding sequence in order to enhance secretion of the polypeptide. However, any signal peptide coding sequence that directs the expressed polypeptide into the secretory pathway of a host cell of choice, i.e., secreted into a culture medium, may be used in the present invention.

Effective signal peptide coding sequences for bacterial host cells are the signal peptide coding sequences obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus oryzae TAKA amylase, Aspergillus niger neutral amylase, Aspergillus niger glucoamylase, Rhizomucor miehei aspartic proteinase, Humicola insolens cellulase, Humicola insolens endoglucanase V, and Humicola lanuginosa lipase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that codes for an amino acid sequence positioned at the amino terminus of a polypeptide. The resultant polypeptide is known as a proenzyme or propolypeptide (or a zymogen in some cases). A propolypeptide is generally inactive and can be converted to a mature active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila laccase (WO 95/33836).

Where both signal peptide and propeptide sequences are present at the amino terminus of a polypeptide, the propeptide sequence is positioned next to the amino terminus of a polypeptide and the signal peptide sequence is positioned next to the amino terminus of the propeptide sequence.

It may also be desirable to add regulatory sequences that allow the regulation of the expression of the polypeptide relative to the growth of the host cell. Examples of regulatory systems are those that cause the expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the TAKA alpha-amylase promoter, Aspergillus niger glucoamylase promoter, and Aspergillus oryzae glucoamylase promoter may be used as regulatory sequences. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the nucleotide sequence encoding the polypeptide would be operably linked with the regulatory sequence.

### Expression Vectors

The various nucleic acids and control sequences described herein may be joined together to produce a recombinant expression vector comprising a polynucleotide encoding a polypeptide having enzyme activity or cellulolytic enhancing activity, a promoter, and transcriptional and translational stop signals. The expression vectors may include one or more convenient restriction sites to allow for insertion or substitution of the polynucleotide sequence encoding the polypeptide at such sites. Alternatively, a polynucleotide encoding such a polypeptide may be expressed by inserting the polynucleotide sequence or a nucleic acid construct comprising the sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide sequence. The choice of the vector will typically depend on the compatibility of the vector with the host cell into which the vector is to be introduced. The vectors may be linear or closed circular plasmids.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vectors preferably contain one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are the dal genes from Bacillus subtilis or Bacillus licheniformis, or markers that confer antibiotic resistance such as ampicillin, kanamycin, chloramphenicol, or tetracycline resistance. Suitable markers for yeast host cells are ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are the amdS and pyrG genes of Aspergillus nidulans or Aspergillus oryzae and the bar gene of Streptomyces hygroscopicus.

The vectors preferably contain an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or nonhomologous recombination. Alternatively, the vector may contain additional nucleotide sequences for directing integration by homologous recombination into the genome of the host cell at a precise location(s) in the chromosome(s). To increase the likelihood of integration at a precise location, the integrational elements should preferably contain a sufficient number of nucleic acids, such as 100 to 10,000 base pairs, preferably 400 to 10,000 base pairs, and most preferably 800 to 16,000 base pairs, which have a high degree of identity to the corresponding target sequence to enhance the probability of homologous recombination. The integrational elements may be any sequence that is homologous with the target sequence in the genome of the host cell. Furthermore, the integrational elements may be non-encoding or encoding nucleotide sequences. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” is defined herein as a nucleotide sequence that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al., 1987, Nucleic Acids Research 15: 9163-9175; WO 00/24883). Isolation of the AMA1 gene and construction of plasmids or vectors comprising the gene can be accomplished according to the methods disclosed in WO 00/24883.

More than one copy of a polynucleotide encoding such a polypeptide may be inserted into the host cell to increase production of the polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors are well known to one skilled in the art (see, e.g., Sambrook et al., 1989, supra).

### Host Cells

Recombinant host cells comprising a polynucleotide encoding a polypeptide having enzyme activity or cellulolytic enhancing activity can be advantageously used in the recombinant production of the polypeptide. A vector comprising such a polynucleotide is introduced into a host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be a unicellular microorganism, e.g., a prokaryote, or a non-unicellular microorganism, e.g., a eukaryote.

The bacterial host cell may be any Gram positive bacterium or a Gram negative bacterium. Gram positive bacteria include, but not limited to, Bacillus, Streptococcus, Streptomyces, Staphylococcus, Enterococcus, Lactobacillus, Lactococcus, Clostridium, Geobacillus, and Oceanobacillus. Gram negative bacteria include, but not limited to, E. coli, Pseudomonas, Salmonella, Campylobacter, Helicobacter, Flavobacterium, Fusobacterium, Ilyobacter, Neisseria, and Ureaplasma.

The bacterial host cell may be any Bacillus cell. Bacillus cells useful in the practice of the present invention include, but are not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

In a preferred aspect, the bacterial host cell is a Bacillus amyloliquefaciens, Bacillus lentus, Bacillus licheniformis, Bacillus stearothermophilus or Bacillus subtilis cell. In a more preferred aspect, the bacterial host cell is a Bacillus amyloliquefaciens cell. In another more preferred aspect, the bacterial host cell is a Bacillus clausii cell. In another more preferred aspect, the bacterial host cell is a Bacillus licheniformis cell. In another more preferred aspect, the bacterial host cell is a Bacillus subtilis cell.

The bacterial host cell may also be any Streptococcus cell. Streptococcus cells useful in the practice of the present invention include, but are not limited to, Streptococcus equisimilis, Streptococcus pyogenes, Streptococcus uberis, and Streptococcus equi subsp. Zooepidemicus cells.

In a preferred aspect, the bacterial host cell is a Streptococcus equisimilis cell. In another preferred aspect, the bacterial host cell is a Streptococcus pyogenes cell. In another preferred aspect, the bacterial host cell is a Streptococcus uberis cell. In another preferred aspect, the bacterial host cell is a Streptococcus equi subsp. Zooepidemicus cell.

The bacterial host cell may also be any Streptomyces cell. Streptomyces cells useful in the practice of the present invention include, but are not limited to, Streptomyces achromogenes, Streptomyces avernitilis, Streptomyces coelicolor, Streptomyces griseus, and Streptomyces lividans cells.

In a preferred aspect, the bacterial host cell is a Streptomyces achromogenes cell. In another preferred aspect, the bacterial host cell is a Streptomyces avermitilis cell. In another preferred aspect, the bacterial host cell is a Streptomyces coeicolor cell. In another preferred aspect, the bacterial host cell is a Streptomyces griseus cell. In another preferred aspect, the bacterial host cell is a Streptomyces lividans cell.

The introduction of DNA into a Bacillus cell may, for instance, be effected by protoplast transformation (see, e.g., Chang and Cohen, 1979, Molecular General Genetics 168: 111-115), by using competent cells (see, e.g., Young and Spizizen, 1961, Journal of Bacteriology 81: 823-829, or Dubnau and Davidoff-Abelson, 1971, Journal of Molecular Biology 56: 209-221), by electroporation (see, e.g., Shigekawa and Dower, 1988, Biotechniques 6: 742-751), or by conjugation (see, e.g., Koehler and Thome, 1987, Journal of Bacteriology 169: 5271-5278). The introduction of DNA into an E coli cell may, for instance, be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may, for instance, be effected by protoplast transformation and electroporation (see, e.g., Gong et al., 2004, Folia Microbiol. (Praha) 49: 399-405), by conjugation (see, e.g., Mazodier et al., 1989, J. Bacteriol. 171: 3583-3585), or by transduction (see, e.g., Burke et al., 2001, Proc. Natl. Acad. Sci. USA 98: 6289-6294). The introduction of DNA into a Pseudomonas cell may, for instance, be effected by electroporation (see, e.g., Choi et al., 2006, J. Microbiol. Methods 64: 391-397) or by conjugation (see, e.g., Pinedo and Smets, 2005, Appl. Environ. Microbiol. 71: 51-57). The introduction of DNA into a Streptococcus cell may, for instance, be effected by natural competence (see, e.g., Perry and Kuramitsu, 1981, Infect. Immun. 32: 1295-1297), by protoplast transformation (see, e.g., Catt and Jollick, 1991, Microbios. 68: 189-2070, by electroporation (see, e.g., Buckley et al., 1999, Appl. Environ. Microbiol. 65: 3800-3804) or by conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

In a preferred aspect, the host cell is a fungal cell. “Fungi” as used herein includes the phyla Ascomycota, Basidiomycota, Chytridiomycota, and Zygomycota (as defined by Hawksworth et al., In, Ainsworth and Bisby's Dictionary of The Fungi, 8th edition, 1995, CAB International, University Press, Cambridge, UK) as well as the Oomycota (as cited in Hawksworth et al., 1995, supra, page 171) and all mitosporic fungi (Hawksworth et al., 1995, supra).

In a more preferred aspect, the fungal host cell is a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, F. A., Passmore, S. M., and Davenport, R. R., eds, Soc. App. Bacteriol. Symposium Series No. 9, 1980).

In an even more preferred aspect, the yeast host cell is a Candida, Hansenula, Kluyveromyces, Pichia, Saccharomyces, Schizosaccharomyces, or Yarrowia cell.

In a most preferred aspect, the yeast host cell is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis cell. In another most preferred aspect, the yeast host cell is a Kluyveromyces lactis cell. In another most preferred aspect, the yeast host cell is a Yarrowia lipolytica cell.

In another more preferred aspect, the fungal host cell is a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

In an even more preferred aspect, the filamentous fungal host cell is an Acremonium, Aspergillus, Aureobasidium, Bjerkandera, Ceriporiopsis, Coprinus, Coriolus, Cryptococcus, Filibasidium, Fusanum, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

In a most preferred aspect, the filamentous fungal host cell is an Aspergillus awamori, Aspergillus fumigatus, Aspergillus foetidus, Aspergillus japonicus, Aspergillus nidulans, Aspergillus niger or Aspergillus oryzae cell. In another most preferred aspect, the filamentous fungal host cell is a Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, or Fusarium venenatum cell. In another most preferred aspect, the filamentous fungal host cell is a Bjerkandera adusta, Ceriporiopsis aneirina, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Coprinus cinereus, Coriolus hirsutus, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238 023 and Yelton et al., 1984, Proceedings of the National Academy of Sciences USA 81: 1470-1474. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, Journal of Bacteriology 153: 163; and Hinnen et al., 1978, Proceedings of the National Academy of Sciences USA 75: 1920.

### Methods of Production

Methods of producing a polypeptide having enzyme activity or cellulolytic enhancing activity, comprise (a) cultivating a cell, which in its wild-type form is capable of producing the polypeptide, under conditions conducive for production of the polypeptide; and (b) recovering the polypeptide.

Alternatively, methods of producing a polypeptide having enzyme activity or cellulolytic enhancing activity, comprise (a) cultivating a recombinant host cell under conditions conducive for production of the polypeptide; and (b) recovering the polypeptide.

In the production methods, the cells are cultivated in a nutrient medium suitable for production of the polypeptide using methods well known in the art. For example, the cell may be cultivated by shake flask cultivation, and small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted into the medium, it can be recovered from cell lysates.

The polypeptides having enzyme or cellulolytic enhancing activity can be detected using the methods described herein or methods known in the art.

The resulting broth may be used as is with or without cellular debris or the polypeptide may be recovered using methods known in the art. For example, the polypeptide may be recovered from the nutrient medium by conventional procedures including, but not limited to, centrifugation, filtration, extraction, spray-drying, evaporation, or precipitation.

The polypeptides may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, J.-C. Janson and Lars Ryden, editors, VCH Publishers, New York, 1989) to obtain substantially pure polypeptides.

The present invention is further described by the following examples that should not be construed as limiting the scope of the invention.

### EXAMPLES

### DNA Sequencing

DNA sequencing was performed using an Applied Biosystems Model 3130X Genetic Analyzer (Applied Biosystems, Foster City, Calif., USA) using dye terminator chemistry (Giesecke et al., 1992, Journal of Virol. Methods 38: 47-60). Sequences were assembled using phred/phrap/consed (University of Washington, Seattle, Wash., USA) with sequence specific primers.

### Media and Solutions

YP medium was composed per liter of 10 g of yeast extract and 20 g of bacto tryptone.

Cellulase-inducing medium was composed per liter of 20 g of cellulose, 10 g of corn steep solids, 1.45 g of (NH₄)₂SO₄, 2.08 g of KH₂PO₄, 0.28 g of CaCl₂, 0.42 g of MgSO₄7H₂O, and 0.42 ml of trace metals solution.

Trace metals solution was composed per liter of 216 g of FeCl₃.6H₂O, 58 g of ZnSO₄.7H₂O, 27 g of MnSO₄.H₂O, 10 g of CuSO₄.5H₂O, 2.4 g of H₃BO₃, and 336 g of citric acid.

STC was composed of 1 M sorbitol, 10 mM CaCl₂, and 10 mM Tris-HCl, pH 7.5.

COVE plates were composed per liter of 342 g of sucrose, 10 ml of COVE salts solution, 10 ml of 1 M acetamide, 10 ml of 1.5 M CsCl, and 25 g of Noble agar.

COVE salts solution was composed per liter of 26 g of KCl, 26 g of MgSO₄, 76.9 of KH₂PO₄, and 50 ml of COVE trace metals solution.

COVE trace metals solution was composed per liter of 0.04 g of Na₂B₄O₇.10H₂O, 0.4 g of CuSO₄.5H₂O, 1.2 g of FeSO₄.7H₂O, 0.7 g of MnSO₄H₂O, 0.8 g of Na₂MoO₂H₂O, and 10 g of ZnSO₄.7H₂O.

COVE2 plates were composed per liter of 30 g of sucrose, 20 ml of COVE salts solution, 25 g of Noble agar, and 10 ml of 1 M acetamide.

PDA plates were composed per liter of 39 grams of potato dextrose agar.

LB medium was composed per liter of 10 g of tryptone, 5 g of yeast extract, and 5 g of sodium chloride.

2× YT-Amp plates were composed per liter of 10 g of tryptone, 5 g of yeast extract, 5 g of sodium chloride, and 15 g of Bacto Agar, followed by 2 ml of a filter-sterilized solution of 50 mg/ml ampicillin after autoclaving.

MDU2BP medium was composed per liter of 45 g of maltose, 1 g of MgSO₄.7H₂O, 1 g of NaCl, 2 g of K₂HSO₄, 12 g of KH₂PO₄, 2 g of urea, and 500 μl of AMG trace metals solution; the pH was adjusted to 5.0 and then filter sterilized with a 0.22 μm filtering unit.

AMG trace metals solution was composed per liter of 14.3 g of ZnSO₄.7H₂O, 2.5 g of CuSO₄.5H₂O, 0.5 g of NiCl₂.6H₂O, 13.8 g of FeSO₄H₂O, 8.5 g of MnSO₄.7H₂O, and 3 g of citric acid.

Minimal medium plates were composed per liter of 6 g of NaNO₃, 0.52 of KCl, 1.52 g of KH₂PO₄, 1 ml of COVE trace metals solution, 20 g of Noble agar, 20 ml of 50% glucose, 2.5 ml of 20% MgSO₄.7H₂O, and 20 ml of biotin stock solution.

Biotin stock solution was composed per liter of 0.2 g of biotin.

SOC medium was composed of 2% tryptone, 0.5% yeast extract, 10 mM NaCl, 2.5 mM KCl, 10 mM MgCl₂, and 10 mM MgSO₄, followed by filter-sterilized glucose to 20 mM after autoclaving.

Mandel's medium was composed per liter of 1.4 g of (NH₄)₂SO₄, 2.0 g of KH₂PO₄, 0.3 g of urea, 0.3 g of CaCl₂, 0.3 g of MgSO₄.7H₂O, 5 mg of FeSO₄.7H₂O, 1.6 mg of MnSO₄.H₂O, 1.4 mg of ZnSO₄.H₂O, and 2 mg of CoCl₂.

### Materials

Phosphoric acid-swollen cellulose (PASC) was prepared from microcrystalline cellulose (AVICEL®; PH101; FMC, Philadelphia, Pa., USA) according to the method of Schulein, 1997, J. Biotechnol. 57: 71-81.

Carboxymethylcellulose (CMC, 7L2 type, 70% substitution) was obtained from Hercules Inc., Wilmington, Del., USA.

Oligomeric proanthocyanidin complex (OPC) was obtained from MASQUELIER'S® Tru-OPCs (Nature's Way Products, Inc., Springville, Utah, USA), containing 75 mg/tablet of dried grape seed extract, of which approximately 65% was OPC and 30% was other polyphenols; inactive ingredients were cellulose, maltodextrin, modified cellulose gum, stearic acid, cellulose, silica, glycerin, etc.). A tablet (0.45 g) was ground by a mortar and pestle and then solubilized in 10 ml water.

Tannic acid (10-galloyl ester of D-glucose), gallic acid, ellagic acid, methyl gallate, glucose pentaacetate (all tannic acid constituent compounds), epicatechin, flavonol (both OPC constituent compounds), 4-hydroxyl-2-methylbenzoic acid, vanillin, coniferyl alcohol, coniferyl aldehyde, ferulic acid, and syringaldehyde (all lignin precursor/constitutent compounds) were obtained from Sigma-Aldrich, St. Louis, Mo., USA. A stock solution of 10 mM tannic acid (corresponding to 100 mM galloyls and 10 mM glucosyl constituents) was prepared in 0.1 M NaOH. Other stock solutions were made in deionized water.

### Example 1

### Preparation of Thermoascus aurantiacus GH61A Polypeptide Having Cellulolytic Enhancing Activity

Thermoascus aurantiacus GH61A polypeptide having cellulolytic enhancing activity was recombinantly produced in Aspergillus oryzae JaL250 according to WO 2005/074656. The recombinantly produced Thermoascus aurantiacus GH61A polypeptide was first concentrated by ultrafiltration using a 10 kDa membrane, buffer exchanged into 20 mM Tris-HCl pH 8.0, and then purified using a 100 ml Q-SEPHAROSE® Big Beads column (GE Healthcare Life Sciences, Piscataway, N.J., USA) with 600 ml of a 0-600 mM NaCl linear gradient in the same buffer. Fractions of 10 ml were collected and pooled based on SDS-PAGE. The pooled fractions (90 ml) were then further purified using a 20 ml MONO Q® column (GE Healthcare Life Sciences, Piscataway, N.J., USA) with 500 ml of a 0-500 mM NaCl linear gradient in the same buffer. Fractions of 6 ml were collected and pooled based on SDS-PAGE. The pooled fractions (24 ml) were concentrated by ultrafiltration using a 10 kDa membrane, and chromatographed using a 320 ml SUPERDEX® 200 SEC column (GE Healthcare Life Sciences, Piscataway, N.J., USA) with isocratic elution of approximately 1.3 liters of 150 mM NaCl-20 mM Tris-HCl pH 8.0. Fractions of 20 ml were collected and pooled based on SDS-PAGE. Protein concentration was determined using a Microplate BCA™ Protein Assay Kit (Pierce, Rockford, Ill., USA).

### Example 2

### Preparation of Trichoderma reesei CEL7A Cellobiohydrolase I

Trichoderma reesei CEL7A cellobiohydrolase I was prepared as described by Ding and Xu, 2004, “Productive cellulase adsorption on cellulose” in Lignocellulose Biodegradation (Saha, B. C. ed.), Symposium Series 889, pp. 154-169, American Chemical Society, Washington, D.C. Protein concentration was determined using a Microplate BCA™ Protein Assay Kit.

### Example 3

### Preparation of Aspergillus oryzae CEL3A Beta-Glucosidase

Aspergillus oryzae CEL3A beta-glucosidase was recombinantly prepared as described in WO 2004/099228, and purified as described by Langston et al., 2006, Biochim. Biophys. Acta Proteins Proteomics 1764: 972-978. Protein concentration was determined using a Microplate BCA™ Protein Assay Kit.

### Example 4

### Preparation of Trichoderma reesei CEL7B Endoglucanase I

The Trichoderma reesei CEL7B endoglucanase I gene was cloned and expressed in Aspergillus oryzae JaL250 as described in WO 2005/067531. Protein concentration was determined using a Microplate BCA™ Protein Assay Kit.

The Trichoderma reesei CEL7B endoglucanase I was desalted and buffer exchanged in 150 mM NaCl-20 mM sodium acetate pH 5.0 using a HIPREP® 26/10 Desalting Column (GE Healthcare Life Sciences, Piscataway, N.J., USA) according to the manufacturer's instructions.

### Example 5

### Preparation of Trichoderma reesei CEL6A Endoglucanase II

The Trichoderma reesei Family GH5A endoglucanase II gene was cloned into an Aspergillus oryzae expression vector as described below.

Two synthetic oligonucleotide primers, shown below, were designed to amplify the endoglucanase II gene from Trichoderma reesei RutC30 genomic DNA. Genomic DNA was isolated using a DNEASY® Plant Maxi Kit (QIAGEN Inc., Valencia, Calif., USA). An IN-FUSION™ PCR Cloning Kit (BD Biosciences, Palo Alto, Calif., USA) was used to clone the fragment directly into pAlLo2 (WO 2004/099228).

(SEQ ID NO: 77)

Forward primer:
5′-ACTGGATTTACCATGAACAAGTCCGTGGCTCCATTGCT-3′

(SEQ ID NO: 78)

Reverse primer:
5′-TCACCTCTAGTTAATTAACTACTTTCTTGCGAGACACG-3′

Bold letters represent coding sequence. The remaining sequence contains sequence identity compared with the insertion sites of pAlLo2.

Fifty picomoles of each of the primers above were used in an amplification reaction containing 200 ng of Trichoderma reesei genomic DNA, 1× Pfx Amplification Buffer (Invitrogen, Carlsbad, Calif., USA), 6 μl of a 10 mM blend of dATP, dTTP, dGTP, and dCTP, 2.5 units of PLATINUM® Pfx DNA polymerase (Invitrogen Corp., Carlsbad, Calif., USA), and 1 μl of 50 mM MgSO₄ (Invitrogen Corp., Carlsbad, Calif., USA) in a final volume of 50 μl. The amplification reaction was incubated in an EPPENDORF® MASTERCYCLER® 5333 (Eppendorf Scientific, Inc., Westbury, N.Y., USA) programmed for 1 cycle at 98° C. for 2 minutes; and 35 cycles each at 94° C. for 30 seconds, 61° C. for 30 seconds, and 68° C. for 1.5 minutes. After the 35 cycles, the reaction was incubated at 68° C. for 10 minutes and then cooled at 10° C. A 1.5 kb PCR product was isolated on a 0.8% GTG® agarose gel (Cambrex Bioproducts, Rutherford, N.J., USA) using 40 mM Tris base-20 mM sodium acetate-1 mM disodium EDTA (TAE) buffer and 0.1 μg of ethidium bromide per ml. The DNA band was visualized with the aid of a DARKREADER™ (Clare Chemical Research, Dolores, Colo., USA). The 1.5 kb DNA band was excised with a disposable razor blade and purified with an ULTRAFREE® DA spin cup (Millipore, Billerica, Mass., USA) according to the manufacturer's instructions.

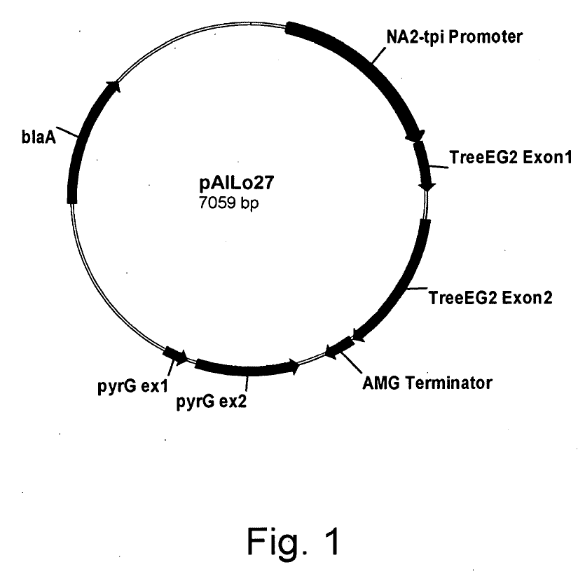

Plasmid pAlLo2 (WO 2004/099228) was linearized by digestion with Nco I and Pac I. The plasmid fragment was purified by gel electrophoresis and ultrafiltration as described above. Cloning of the purified PCR fragment into the linearized and purified pAlLo2 vector was performed with an IN-FUSION™ PCR Cloning Kit. The reaction (20 μl) contained of 1×IN-FUSION™ Buffer (BD Biosciences, Palo Alto, Calif., USA), 1×BSA (BD Biosciences, Palo Alto, Calif., USA), 1 μl of IN-FUSION™ enzyme (diluted 1:10) (BD Biosciences, Palo Alto, Calif., USA), 100 ng of pAlLo2 digested with Nco I and Pac I, and 100 ng of the Trichoderma reesei CEL6A endoglucanase II PCR product. The reaction was incubated at room temperature for 30 minutes. A 2 μl sample of the reaction was used to transform E. coli XL10 SOLOPACK® Gold cells (Stratagene, La Jolla, Calif., USA) according to the manufacturers instructions. After a recovery period, two 100 μl aliquots from the transformation reaction were plated onto 150 mm 2×YT plates supplemented with 100 μg of ampicillin per ml. The plates were incubated overnight at 37° C. A set of 3 putative recombinant clones was recovered the selection plates and plasmid DNA was prepared from each one using a BIOROBOT® 9600 (QIAGEN, Inc., Valencia, Calif., USA). Clones were analyzed by Pci I/BspLU11I restriction digestion. One clone with the expected restriction digestion pattern was then sequenced to confirm that there were no mutations in the cloned insert. Clone #3 was selected and designated pAlLo27 (FIG. 1).

Aspergillus oryzae JaL250 (WO 99/61651) protoplasts were prepared according to the method of Christensen et al., 1988, Bio/Technology 6: 1419-1422. Five micrograms of pAlLo27 (as well as pAlLo2 as a control) were used to transform Aspergillus oryzae JaL250 protoplasts.

The transformation of Aspergillus oryzae JaL950 with pAlLo27 yielded about 50 transformants. Eleven transformants were isolated to individual PDA plates and incubated for five days at 34° C.

Confluent spore plates were washed with 3 ml of 0.01% TWEEN® 80 and the spore suspension was used to inoculate 25 ml of MDU2BP medium in 125 ml glass shake flasks. Transformant cultures were incubated at 34° C. with constant shaking at 200 rpm. At day five post-inoculation, cultures were centrifuged at 6000×g and their supernatants collected. Five microliters of each supernatant were mixed with an equal volume of 2× loading buffer (10% beta-mercaptoethanol) and loaded onto a 1.5 mm 8%-16% Tris-Glycine SDS-PAGE gel and stained with SIMPLYBLUE™ SafeStain (Invitrogen Corp., Carlsbad, Calif., USA). SDS-PAGE profiles of the culture broths showed that ten out of eleven transformants produced a new protein band of approximately 45 kDa. Transformant number 1, designated Aspergillus oryzae JaL250AILo27, was cultivated in a fermentor.

Shake flask medium was composed per liter of 50 g of sucrose, 10 g of KH₂PO₄, 0.5 g of CaCl₂, 2 g of MgSO₄.7H₂O, 2 g of K₂SO₄, 2 g of urea, 10 g of yeast extract, 2 g of citric acid, and 0.5 ml of trace metals solution. Trace metals solution was composed per liter of 13.8 g of FeSO₄.7H₂O, 14.3 g of ZnSO₄.7H₂O, 8.5 g of MnSO₄—H₂O, 2.5 g of CuSO₄.5H₂O, and 3 g of citric acid.

One hundred ml of shake flask medium was added to a 500 ml shake flask. The shake flask was inoculated with two plugs from a solid plate culture and incubated at 34° C. on an orbital shaker at 200 rpm for 24 hours. Fifty ml of the shake flask broth was used to inoculate a 3 liter fermentation vessel.

Fermentation batch medium was composed per liter of 10 g of yeast extract, 24 g of sucrose, 5 g of (NH₄)₂SO₄, 2 g of KH₂PO₄, 0.5 g of CaCl₂.2H₂O, 2 g of MgSO₄.7H₂O, 19 of citric acid, 2 g of K₂SO₄, 0.5° ml of anti-foam, and 0.5 ml of trace metals solution. Trace metals solution was composed per liter of 13.8 g of FeSO₄.7H₂O, 14.3 g of ZnSO₄.7H₂O, 8.5 g of MnSO₄.H₂O, 2.5 g of CuSO₄.5H₂O, and 3 g of citric acid. Fermentation feed medium was composed of maltose.

A total of 1.8 liters of the fermentation batch medium was added to a three liter glass jacketed fermentor (Applikon Biotechnology, Inc. Foster City, Calif., USA). Fermentation feed medium was dosed at a rate of 0 to 4.4 g/l/hr for a period of 185 hours. The fermentation vessel was maintained at a temperature of 34° C. and pH was controlled using an APPLIKON® 1030 control system (Applikon Biotechnology, Inc. Foster City, Calif., USA) to a set-point of 6.1+/−0.1. Air was added to the vessel at a rate of 1 vvm and the broth was agitated by Rushton impeller rotating at 1100 to 1300 rpm. At the end of the fermentation, whole broth was harvested from the vessel and centrifuged at 3000×g to remove the biomass. The supernatant was sterile filtered and stored at 5 to 10° C.

The supernatant was desalted and buffer-exchanged in 20 mM sodium acetate-150 mM NaCl pH 5.0 using a HIPREP® 26/10 Desalting column according to the manufacturer's instructions. Protein concentration was determined using a Microplate BCA™ Protein Assay Kit.

### Example 6

### Preparation of Trichoderma reesei CEL6A Cellobiohydrolase II

The Trichoderma reesei CEL6A cellobiohydrolase II gene was isolated from Trichoderma reesei RutC30 as described in WO 2005/056772.

The Trichoderma reesei CEL6A cellobiohydrolase II gene was expressed in Fusarium venenatum using pEJG61 as an expression vector according to the procedures described in U.S. Published Application No. 20060156437. Fermentation was performed as described in U.S. Published Application No. 20060156437. Protein concentration was determined using a Microplate BCA™ Protein Assay Kit.

The Trichoderma reesei CEL6A cellobiohydrolase II was desalted and buffer-exchanged into 20 mM sodium acetate-150 mM NaCl pH 5.0 using a HIPREP® 26/10 Desalting column according to the manufacturer's instructions.

### Example 7

### Construction of pMJ04 Expression Vector

Expression vector pMJ04 was constructed by PCR amplifying the Trichoderma reesei cellobiohydrolase 1 gene (cbh1, CEL7A) terminator from Trichoderma reesei RutC30 genomic DNA using primers 993429 (antisense) and 993428 (sense) shown below. The antisense primer was engineered to have a Pac I site at the 5′-end and a Spe I site at the 3′-end of the sense primer.

(SEQ ID NO: 79)

Primer 993429 (antisense):
5′-AACGTTAATTAAGGAATCGTTTTGTGTTT-3′

(SEQ ID NO: 80)

Primer 993428 (sense):
5′-AGTACTAGTAGCTCCGTGGCGAAAGCCTG-3′

Trichoderma reesei RutC30 genomic DNA was isolated using a DNEASY® Plant Maxi Kit.

The amplification reactions (50 μl) were composed of 1× ThermoPol Reaction Buffer (New England Biolabs, Beverly, Mass., USA), 0.3 mM dNTPs, 100 ng of Trichoderma reesei RutC30 genomic DNA, 0.3 μM primer 993429, 0.3 μM primer 993428, and 2 units of Vent DNA polymerase (New England Biolabs, Beverly, Mass., USA). The reactions were incubated in an EPPENDORF® MASTERCYCLER® 5333 programmed for 5 cycles each for 30 seconds at 94° C., 30 seconds at 50° C., and 60 seconds at 72° C., followed by 25 cycles each for 30 seconds at 94° C., 30 seconds at 65° C., and 120 seconds at 72° C. (5 minute final extension). The reaction products were isolated by 1.0% agarose gel electrophoresis using TAE buffer where a 229 bp product band was excised from the gel and purified using a QIAQUICK® Gel Extraction Kit (QIAGEN Inc., Valencia, Calif., USA) according to the manufacturer's instructions.

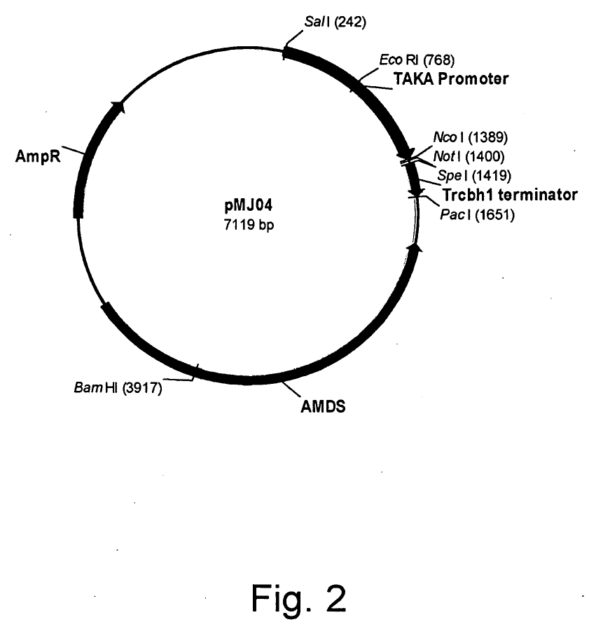

The resulting PCR fragment was digested with Pac I and Spe I and ligated into pAlLo1 (WO 05/067531) digested with the same restriction enzymes using a Rapid DNA Ligation Kit (Roche, Indianapolis, Ind., USA) to generate pMJ04 (FIG. 2).

### Example 8

### Construction of pCaHj568

Plasmid pCaHj568 was constructed from pCaHj170 (U.S. Pat. No. 5,763,254) and pMT2188. Plasmid pCaHj170 comprises the Humicola insolens endoglucanase V (CEL45A) full-length coding region (SEQ ID NO: 11, which encodes the amino acid sequence of SEQ ID NO: 12). Construction of pMT2188 was initiated by PCR amplifying the pUC19 origin of replication from pCaHj483 (WO 98/00529) using primers 142779 and 142780 shown below. Primer 142780 introduces a Bbu I site in the PCR fragment.

(SEQ ID NO: 81)

Primer 142779:
5′-TTGAATTGAAAATAGATTGATTTAAAACTTC-3′

(SEQ ID NO: 82)

Primer 142780:
5′-TTGCATGCGTAATCATGGTCATAGC-3′

An EXPAND® PCR System (Roche Molecular Biochemicals, Basel, Switzerland) was used following the manufacturer's instructions for this amplification. PCR products were separated on an agarose gel and an 1160 bp fragment was isolated and purified using a Jetquick Gel Extraction Spin Kit (Genomed, Wielandstr, Germany).

The URA3 gene was amplified from the general Saccharomyces cerevisiae cloning vector pYES2 (Invitrogen, Carlsbad, Calif., USA) using primers 140288 and 142778 shown below using an EXPAND® PCR System. Primer 140288 introduced an Eco RI site into the PCR fragment.

(SEQ ID NO: 83)

Primer 140288:
5′-TTGAATTCATGGGTAATAACTGATAT-3′

(SEQ ID NO: 84)

Primer 142778:
5′-AAATCAATCTATTTTCAATTCAATTCATCATT-3′

PCR products were separated on an agarose gel and an 1126 bp fragment was isolated and purified using a Jetquick Gel Extraction Spin Kit.

The two PCR fragments were fused by mixing and amplified using primers 142780 and 140288 shown above by the overlap splicing method (Horton et al., 1989, Gene 77: 61-68). PCR products were separated on an agarose gel and a 2263 bp fragment was isolated and purified using a Jetquick Gel Extraction Spin Kit.

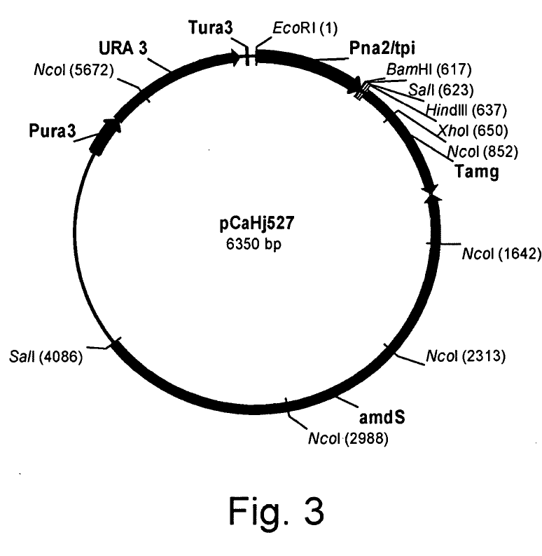

The resulting fragment was digested with Eco RI and Bbu I and ligated using standard protocols to the largest fragment of pCaHj483 digested with the same restriction enzymes. The ligation mixture was transformed into pyrF-negative E. coli strain DB6507 (ATCC 35673) made competent by the method of Mandel and Higa, 1970, J. Mol. Biol. 45: 154. Transformants were selected on solid M9 medium (Sambrook et al., 1989, Molecular Cloning, A Laboratory Manual, 2nd edition, Cold Spring Harbor Laboratory Press) supplemented per liter with 1 g of casamino acids, 500 μg of thiamine, and 10 mg of kanamycin. A plasmid from one transformant was isolated and designated pCaHj527 (FIG. 3).

The NA2-tpi promoter present on pCaHj527 was subjected to site-directed mutagenesis by PCR using an EXPAND® PCR System according to the manufacturer's instructions. Nucleotides 134-144 were converted from GTACTAAAACC (SEQ ID NO: 85) to CCGTTAAATTT (SEQ ID NO: 86) using mutagenic primer 141223 shown below.

| | |
|---|---|
| Primer 141223: | |
| 5′-GGATGCTGTTGACTCCGGAAATTTAACGGTTTGGTCTTGCATCCC-3′ | (SEQ ID NO: 87) |

Nucleotides 423-436 were converted from ATGCAATTTAAACT (SEQ ID NO: 88) to CGGCAATTTAACGG (SEQ ID NO: 89) using mutagenic primer 141222 shown below.

Primer 141222:

(SEQ ID NO: 90)

5′-GGTATTGTCCTGCAGACGGCAATTTAACGGCTTCTGCGAATCGC-3′

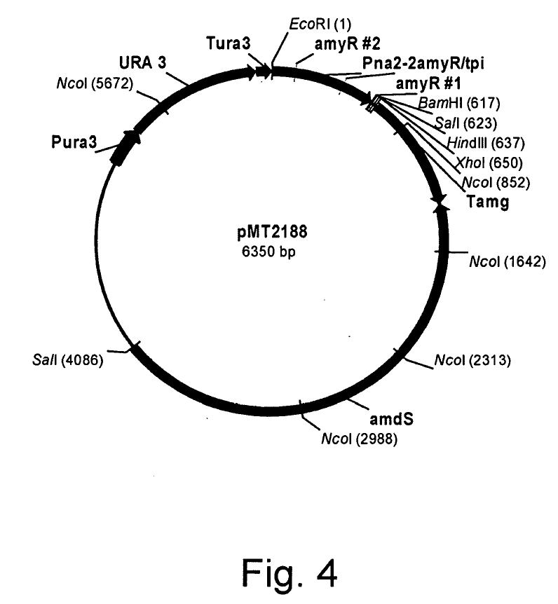

The resulting plasmid was designated pMT2188 (FIG. 4).

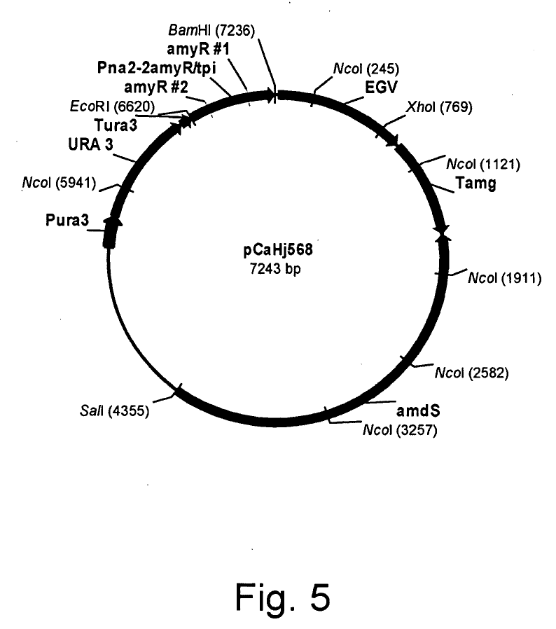

The Humicola insolens endoglucanase V coding region was transferred from pCaHj170 as a Bam HI-Sal I fragment into pMT2188 digested with Bam HI and Xho I to generate pCaHj568 (FIG. 5). Plasmid pCaHj568 comprises a mutated NA2-tpi promoter operably linked to the Humicola insolens endoglucanase V full-length coding sequence.

### Example 9

### Construction of pMJ05

Plasmid pMJ05 was constructed by PCR amplifying the 915 bp Humicola insolens endoglucanase V full-length coding region from pCaHj568 using primers HiEGV-F and HiEGV-R shown below.

Primer HiEGV-F (sense):

(SEQ ID NO: 91)

5′-AAGCTTAAGCATGCGTTCCTCCCCCCTCC-3′
Primer HiEGV-R (antisense):

(SEQ ID NO: 92)

5′-CTGCAGAATTCTACAGGCACTGATGGTACCAG-3′

The amplification reactions (50 μl) were composed of 1× ThermoPol Reaction Buffer, 0.3 mM dNTPs, 10 ng/μl of pCaHj568, 0.3 μM HiEGV-F primer, 0.3 μM HiEGV-R primer, and 2 units of Vent DNA polymerase. The reactions were incubated in an EPPENDORF® MASTERCYCLER® 5333 programmed for 5 cycles each for 30 seconds at 94° C., 30 seconds at 50° C., and 60 seconds at 72° C., followed by 25 cycles each for 30 seconds at 94° C., 30 seconds at 65° C., and 120 seconds at 72° C. (5 minute final extension). The reaction products were isolated on a 1.0% agarose gel using TAE buffer where a 937 bp product band was excised from the gel and purified using a QIAQUICK® Gel Extraction Kit according to the manufacturer's instructions.

The 937 bp purified fragment was used as template DNA for subsequent amplifications with the following primers:

Primer HiEGV-R (antisense):

(SEQ ID NO: 93)

5′-CTGCAGAATTCTACAGGCACTGATGGTACCAG-3′
Primer HiEGV-F-overlap (sense):

(SEQ ID NO: 94)

5′-ACCGCGGACTGCGCATCATGCGTTCCTCCCCCCTCC-3′

Primer sequences in italics are homologous to 17 bp of the Trichoderma reesei cellobiohydrolase I gene (cbh1) promoter and underlined primer sequences are homologous to 29 bp of the Humicola insolens endoglucanase V coding region. A 36 bp overlap between the promoter and the coding sequence allowed precise fusion of a 994 bp fragment comprising the Trichoderma reesei cbh1 promoter to the 918 bp fragment comprising the Humicola insolens endoglucanase V coding region.

The amplification reactions (50 μl) were composed of 1× ThermoPol Reaction Buffer, 0.3 mM dNTPs, 1 μl of the purified 937 bp PCR fragment, 0.3 μM HiEGV-F-overlap primer, 0.3 μM HiEGV-R primer, and 2 units of Vent DNA polymerase. The reactions were incubated in an EPPENDORF® MASTERCYCLER® 5333 programmed for 5 cycles each for 30 seconds at 94° C., 30 seconds at 50° C., and 60 seconds at 72° C., followed by 25 cycles each for 30 seconds at 94° C., 30 seconds at 65° C., and 120 seconds at 72° C. (5 minute final extension). The reaction products were isolated on a 1.0% agarose gel using TAE buffer where a 945 bp product band was excised from the gel and purified using a QIAQUICK® Gel Extraction Kit according to the manufacturer's instructions.

A separate PCR was performed to amplify the Trichoderma reesei cbh1 promoter sequence extending from 994 bp upstream of the ATG start codon of the gene from Trichoderma reesei RutC30 genomic DNA using the primers shown below (the sense primer was engineered to have a Sal I restriction site at the 5′-end). Trichoderma reesei RutC30 genomic DNA was isolated using a DNEASY® Plant Maxi Kit.

Primer TrCBHIpro-F (sense):

(SEQ ID NO: 95)

5′-AAACGTCGACCGAATGTAGGATTGTTATC-3′
Primer TrCBHIpro-R (antisense):

(SEQ ID NO: 96)

5′-GATGCGCAGTCCGCGGT-3′

The amplification reactions (50 μl) were composed of 1× ThermoPol Reaction Buffer, 0.3 mM dNTPs, 100 ng/μl Trichoderma reesei RutC30 genomic DNA, 0.3 μM TrCBHIpro-F primer, 0.3 μM TrCBHIpro-R primer, and 2 units of Vent DNA polymerase. The reactions were incubated in an EPPENDORF® MASTERCYCLER® 5333 programmed for 30 cycles each for 30 seconds at 94° C., 30 seconds at 55° C., and 120 seconds at 72° C. (5 minute final extension). The reaction products were isolated on a 1.0% agarose gel using TAE buffer where a 998 bp product band was excised from the gel and purified using a QIAQUICK® Gel Extraction Kit according to the manufacturer's instructions.

The purified 998 bp PCR fragment was used as template DNA for subsequent amplifications using the primers shown below.

Primer TrCBHIpro-F:

(SEQ ID NO: 97)

5′-AAACGTCGACCGAATGTAGGATTGTTATC-3′
Primer TrCBHIpro-R-overlap:

(SEQ ID NO: 98)

5′-GGAGGGGGGAGGAACGCATGATGCGCAGTCCGCGGT-3′

Sequences in italics are homologous to 17 bp of the Trichoderma reesei cbh1 promoter and underlined sequences are homologous to 29 bp of the Humicola insolens endoglucanase V coding region. A 36 bp overlap between the promoter and the coding sequence allowed precise fusion of the 994 bp fragment comprising the Trichoderma reesei cbh1 promoter to the 918 bp fragment comprising the Humicola insolens endoglucanase V full-length coding region.

The amplification reactions (50 μl) were composed of 1× ThermoPol Reaction Buffer, 0.3 mM dNTPs, 1 μl of the purified 998 bp PCR fragment, 0.3 μM TrCBH1pro-F primer, 0.3 μM TrCBH1pro-R-overlap primer, and 2 units of Vent DNA polymerase. The reactions were incubated in an EPPENDORF® MASTERCYCLER® 5333 programmed for 5 cycles each for 30 seconds at 94° C., 30 seconds at 50° C., and 60 seconds at 72° C., followed by 25 cycles each for 30 seconds at 94° C., 30 seconds at 65° C., and 120 seconds at 72° C. (5 minute final extension). The reaction products were isolated on a 1.0% agarose gel using TAE buffer where a 1017 bp product band was excised from the gel and purified using a QIAQUICK® Gel Extraction Kit according to the manufacturer's instructions.

The 1017 bp Trichoderma reesei cbh1 promoter PCR fragment and the 945 bp Humicola insolens endoglucanase V PCR fragment were used as template DNA for subsequent amplification using the following primers to precisely fuse the 994 bp cbh1 promoter to the 918 bp endoglucanase V full-length coding region using overlapping PCR.

Primer TrCBHIpro-F:

(SEQ ID NO: 99)

5′-AAACGTCGACCGAATGTAGGATTGTTATC-3′
Primer HiEGV-R:

(SEQ ID NO: 100)

5′-CTGCAGAATTCTACAGGCACTGATGGTACCAG-3′

The amplification reactions (50 μl) were composed of 1× ThermoPol Reaction Buffer, 0.3 mM dNTPs, 0.3 μM TrCBHIpro-F primer, 0.3 μM HiEGV-R primer, and 2 units of Vent DNA polymerase. The reactions were incubated in an EPPENDORF® MASTERCYCLER® 5333 programmed for 5 cycles each for 30 seconds at 94° C., 30 seconds at 50° C., and 60 seconds at 72° C., followed by 25 cycles each for 30 seconds at 94° C., 30 seconds at 65° C., and 120 seconds at 72° C. (5 minute final extension). The reaction products were isolated on a 1.0% agarose gel using TAE buffer where a 1926 bp product band was excised from the gel and purified using a QIAQUICK® Gel Extraction Kit according to the manufacturer's instructions.

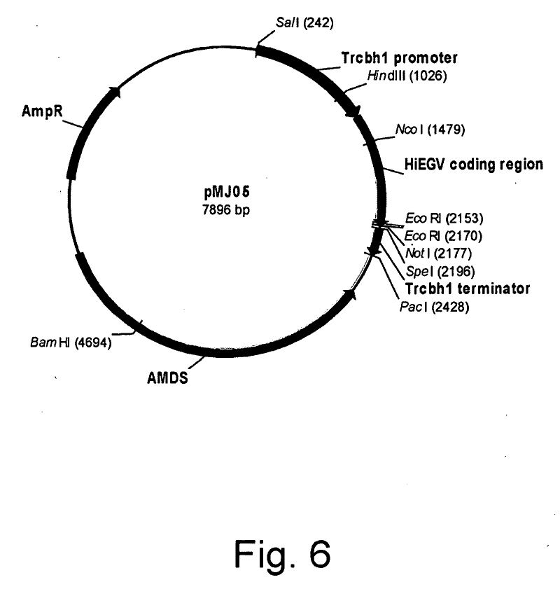

The resulting 1926 bp fragment was cloned into a pCR®-Blunt-II-TOPO® vector (Invitrogen, Carlsbad, Calif., USA) using a ZEROBLUNT® TOPO® PCR Cloning Kit (Invitrogen, Carlsbad, Calif., USA) following the manufacturer's protocol. The resulting plasmid was digested with Not I and Sal I and the 1926 bp fragment was gel purified using a QIAQUICKO Gel Extraction Kit and ligated using T4 DNA ligase (Roche, Indianapolis, Ind., USA) into pMJ04, which was also digested with the same two restriction enzymes, to generate pMJ05 (FIG. 6). Plasmid pMJ05 comprises the Trichoderma reesei cellobiohydrolase I promoter and terminator operably linked to the Humicola insolens endoglucanase V full-length coding sequence.

### Example 10

### Construction of pSMai130 Expression Vector

A 2586 bp DNA fragment spanning from the ATG start codon to the TAA stop codon of the Aspergillus oryzae beta-glucosidase full-length coding sequence (SEQ ID NO: 47 for cDNA sequence and SEQ ID NO: 48 for the deduced amino acid sequence; E. coli DSM 14240) was amplified by PCR from pJaL660 (WO 2002/095014) as template with primers 993467 (sense) and 993456 (antisense) shown below. A Spe I site was engineered at the 5′ end of the antisense primer to facilitate ligation. Primer sequences in italics are homologous to 24 bp of the Trichoderma reesei cbh1 promoter and underlined sequences are homologous to 22 bp of the Aspergillus oryzae beta-glucosidase coding region.

| | |
|---|---|
| Primer 993467: | |
| 5′-ATAGTCAACCGCGGACTGCGCATCATGAAGCTTGGTTGGATCGAGG-3′ | (SEQ ID NO: 101) |
| Primer 993456: | |
| 5′-ACTAGTTTACTGGGCCTTAGGCAGCG-3′ | (SEQ ID NO: 102) |

The amplification reactions (50 μl) were composed of Pfx Amplification Buffer (Invitrogen, Carlsbad, Calif., USA), 0.25 mM dNTPs, 10 ng of pJaL660, 6.4 μM primer 993467, 3.2 μM primer 993456, 1 mM MgCl₂, and 2.5 units of Pfx DNA polymerase (Invitrogen, Carlsbad, Calif., USA). The reactions were incubated in an EPPENDORF® MASTERCYCLER® 5333 programmed for 30 cycles each for 1 minute at 94° C., 1 minute at 55° C., and 3 minutes at 72° C. (15 minute final extension). The reaction products were isolated on a 1.0% agarose gel using TAE buffer where a 2586 bp product band was excised from the gel and purified using a QIAQUICK® Gel Extraction Kit according to the manufacturer's instructions.

A separate PCR was performed to amplify the Trichoderma reesei cbh1 promoter sequence extending from 1000 bp upstream of the ATG start codon of the gene, using primer 993453 (sense) and primer 993463 (antisense) shown below to generate a 1000 bp PCR fragment.

| | |
|---|---|
| Primer 993453: | |
| 5′-GTCGACTCGAAGCCCGAATGTAGGAT-3′ | (SEQ ID NO: 103) |
| Primer 993463: | |
| 5′-CCTCGATCCAACCAAGCTTCATGATGCGCAGTCCGCGGTTGACTA-3′ | (SEQ ID NO: 104) |

Primer sequences in italics are homologous to 24 bp of the Trichoderma reesei cbh1 promoter and underlined primer sequences are homologous to 22 bp of the Aspergillus oryzae beta-glucosidase full-length coding region. The 46 bp overlap between the promoter and the coding sequence allowed precise fusion of the 1000 bp fragment comprising the Trichoderma reesei cbh1 promoter to the 2586 bp fragment comprising the Aspergillus oryzae beta-glucosidase coding region.

The amplification reactions (50 μl) were composed of Pfx Amplification Buffer, 0.25 mM dNTPs, 100 ng of Trichoderma reesei RutC30 genomic DNA, 6.4 μM primer 993453, 3.2 μM primer 993463, 1 mM MgCl₂, and 2.5 units of Pfx DNA polymerase. The reactions were incubated in an EPPENDORF® MASTERCYCLER® 5333 programmed for 30 cycles each for 1 minute at 94° C., 1 minute at 55° C., and 3 minutes at 72° C. (15 minute final extension). The reaction products were isolated on a 1.0% agarose gel using TAE buffer where a 1000 bp product band was excised from the gel and purified using a QIAQUICK® Gel Extraction Kit according to the manufacturer's instructions.

The purified fragments were used as template DNA for subsequent amplification by overlapping PCR using primer 993453 (sense) and primer 993456 (antisense) shown above to precisely fuse the 1000 bp fragment comprising the Trichoderma reesei cbh1 promoter to the 2586 bp fragment comprising the Aspergillus oryzae beta-glucosidase full-length coding region.

The amplification reactions (50 μl) were composed of Pfx Amplification Buffer, 0.25 mM dNTPs, 6.4 μM primer 99353, 3.2 μM primer 993456, 1 mM MgCl₂, and 2.5 units of Pfx DNA polymerase. The reactions were incubated in an EPPENDORF® MASTERCYCLER® 5333 programmed for 30 cycles each for 1 minute at 94° C., 1 minute at 60° C., and 4 minutes at 72° C. (15 minute final extension).

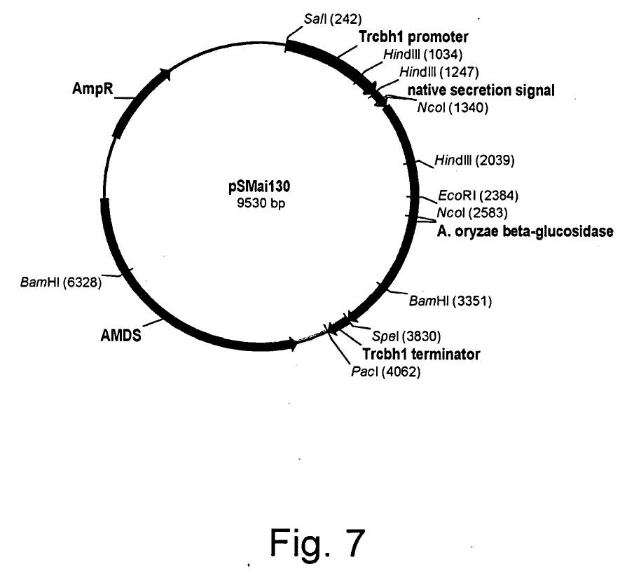

The resulting 3586 bp fragment was digested with Sal I and Spe I and ligated into pMJ04, digested with the same two restriction enzymes, to generate pSMai130 (FIG. 7). Plasmid pSMai130 comprises the Trichoderma reesei cellobiohydrolase I gene promoter and terminator operably linked to the Aspergillus oryzae native beta-glucosidase signal sequence and coding sequence (i.e., full-length Aspergillus oryzae beta-glucosidase coding sequence).

### Example 11

### Construction of pSMai135

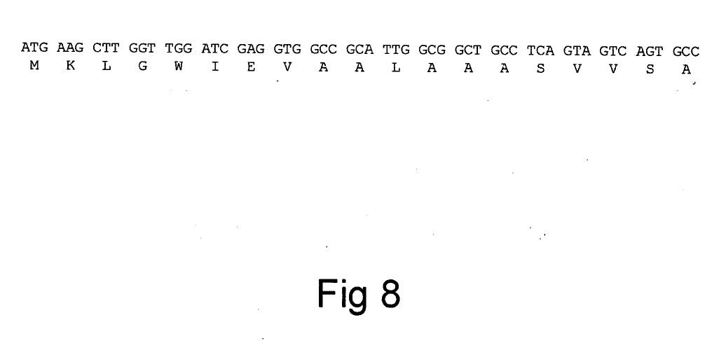

The Aspergillus oryzae beta-glucosidase mature coding region (minus the native signal sequence, see FIG. 8; SEQ ID NOs: 105 and 106 for signal peptide and coding sequence thereof) from Lys-20 to the TAA stop codon was PCR amplified from pJaL660 as template with primer 993728 (sense) and primer 993727 (antisense) shown below.

| | |
|---|---|
| Primer 993728: | |
| 5′-TGCCGGTGTTGGCCCTTGCCAAGGATGATCTCGCGTACTCCC-3′ | (SEQ ID NO: 107) |
| Primer 993727: | |
| 5′-GACTAGTCTTACTGGGCCTTAGGCAGCG-3′ | (SEQ ID NO: 108) |

Sequences in italics are homologous to 20 bp of the Humicola insolens endoglucanase V signal sequence and sequences underlined are homologous to 22 bp of the Aspergillus oryzae beta-glucosidase coding region. A Spe I site was engineered into the 5′ end of the antisense primer.

The amplification reactions (50 μl) were composed of Pfx Amplification Buffer, 0.25 mM dNTPs, 10 ng/μl of pJaL660, 6.4 μM primer 993728, 3.2 μM primer 993727, 1 mM MgCl₂, and 2.5 units of Pfx DNA polymerase. The reactions were incubated in an EPPENDORF® MASTERCYCLER® 5333 programmed for 30 cycles each for 1 minute at 94° C., 1 minute at 55° C., and 3 minutes at 72° C. (15 minute final extension). The reaction products were isolated on a 1.0% agarose gel using TAE buffer where a 2523 bp product band was excised from the gel and purified using a QIAQUICK® Gel Extraction Kit according to the manufacturer's instructions.

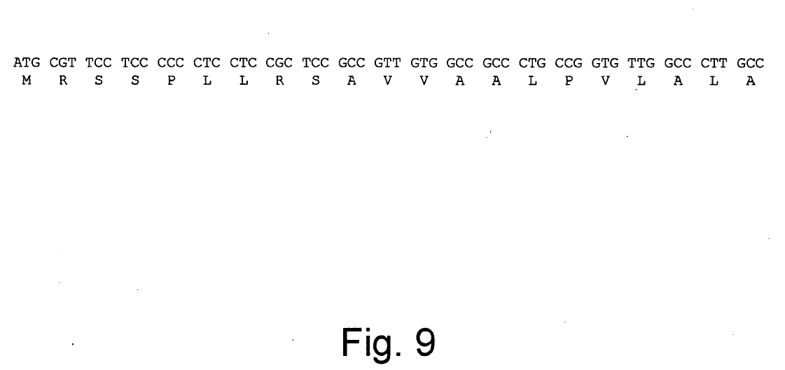

A separate PCR amplification was performed to amplify 1000 bp of the Trichoderma reesei cbh1 promoter and 63 bp of the Humicola insolens endoglucanase V signal sequence (ATG start codon to Ala-21, FIG. 9, SEQ ID NOs: 109 and 110) using primer 993724 (sense) and primer 993729 (antisense) shown below.

Primer 993724:

(SEQ ID NO: 111)

5′-ACGCGTCGACCGAATGTAGGATTGTTATCC-3′
Primer 993729:

(SEQ ID NO: 112)

5′-GGGAGTACGCGAGATCATCCTTGGCAAGGGCCAACACCGGCA-3′

Primer sequences in italics are homologous to 20 bp of the Humicola insolens endoglucanase V signal sequence and underlined primer sequences are homologous to the 22 bp of the Aspergillus oryzae beta-glucosidase coding region.

Plasmid pMJ05, which comprises the Humicola insolens endoglucanase V coding region under the control of the cbh1 promoter, was used as template to generate a 1063 bp fragment comprising the Trichoderma reesei cbh1 promoter and Humicola insolens endoglucanase V signal sequence fragment. A 42 bp of overlap was shared between the Trichoderma reesei cbh1 promoter and Humicola insolens endoglucanase V signal sequence and the Aspergillus oryzae beta-glucosidase mature coding sequence to provide a perfect linkage between the promoter and the ATG start codon of the 2523 bp Aspergillus oryzae beta-glucosidase coding region.

The amplification reactions (50 μl) were composed of Pfx Amplification Buffer, 0.25 mM dNTPs, 10 ng/μl of pMJ05, 6.4 μM primer 993728, 3.2 μM primer 993727, 1 mM MgCl₂, and 2.5 units of Pfx DNA polymerase. The reactions were incubated in an EPPENDORF® MASTERCYCLER® 5333 programmed for 30 cycles each for 1 minute at 94° C., 1 minute at 60° C., and 4 minutes at 72° C. (15 minute final extension). The reaction products were isolated on a 1.0% agarose gel using TAE buffer where a 1063 bp product band was excised from the gel and purified using a QIAQUICK® Gel Extraction Kit according to the manufacturer's instructions.

The purified overlapping fragments were used as templates for amplification employing primer 993724 (sense) and primer 993727 (antisense) described above to precisely fuse the 1063 bp fragment comprising the Trichoderma reesei cbh1 promoter and Humicola insolens endoglucanase V signal sequence to the 2523 bp fragment comprising the Aspergillus oryzae beta-glucosidase mature coding region frame by overlapping PCR.

The amplification reactions (50 μl) were composed of Pfx Amplification Buffer, 0.25 mM dNTPs, 6.4 μM primer 993724, 3.2 μM primer 993727, 1 mM MgCl₂, and 2.5 units of Pfx DNA polymerase. The reactions were incubated in an EPPENDORF® MASTERCYCLER® 5333 programmed for 30 cycles each for 1 minute at 94° C., 1 minute at 60° C., and 4 minutes at 72° C. (15 minute final extension). The reaction products were isolated on a 1.0% agarose gel using TAE buffer where a 3591 bp product band was excised from the gel and purified using a QIAQUICK® Gel Extraction Kit according to the manufacturer's instructions.

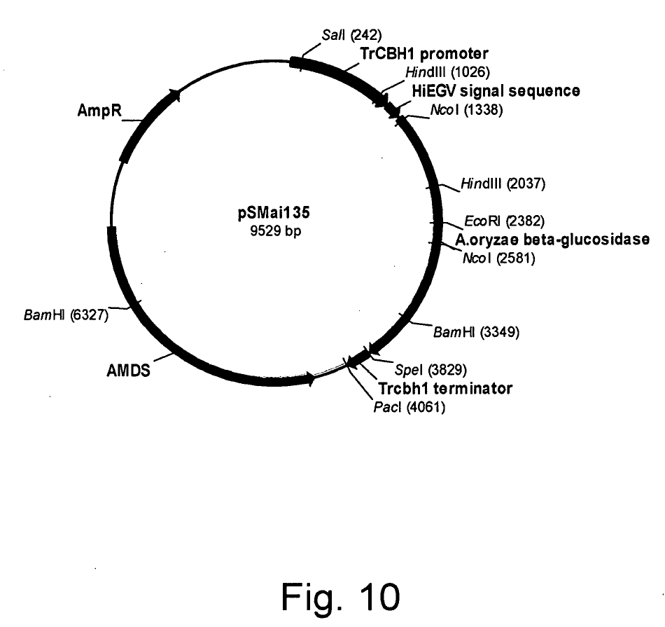

The resulting 3591 bp fragment was digested with Sal I and Spe I and ligated into pMJ04 digested with the same restriction enzymes to generate pSMai135 (FIG. 10). Plasmid pSMai135 comprises the Trichoderma reesei cellobiohydrolase I gene promoter and terminator operably linked to the Humicola insolens endoglucanase V signal sequence and the Aspergillus oryzae beta-glucosidase mature coding sequence.

### Example 12

### Expression of Aspergillus oryzae Beta-Glucosidase with the Humicola insolens Endoglucanase V Secretion Signal

Plasmid pSMai135 encoding the mature Aspergillus oryzae beta-glucosidase linked to the Humicola insolens endoglucanase V secretion signal (FIG. 9) was introduced into Trichoderma reesei RutC30 by PEG-mediated transformation (Penttila et al., 1987, Gene 61 155-164). The plasmid contained the Aspergillus nidulans amdS gene to enable transformants to grow on acetamide as the sole nitrogen source.

Trichoderma reesei RutC30 was cultivated at 27° C. and 90 rpm in 25 ml of YP medium supplemented with 2% (w/v) glucose and 10 mM uridine for 17 hours. Mycelia were collected by filtration using a Vacuum Driven Disposable Filtration System (Millipore, Bedford, Mass., USA) and washed twice with deionized water and twice with 1.2 M sorbitol. Protoplasts were generated by suspending the washed mycelia in 20 ml of 1.2 M sorbitol containing 15 mg of GLUCANEX® (Novozymes A/S, Bagsværd, Denmark) per ml and 0.36 units of chitinase (Sigma Chemical Co., St. Louis, Mo., USA) per ml and incubating for 15-25 minutes at 34° C. with gentle shaking at 90 rpm. Protoplasts were collected by centrifuging for 7 minutes at 400×g and washed twice with cold 1.2 M sorbitol. The protoplasts were counted using a haemacytometer and re-suspended in STC to a final concentration of 1×10⁸ protoplasts per ml. Excess protoplasts were stored in a Cryo 1° C. Freezing Container (Nalgene, Rochester, N.Y., USA) at −80° C.

Approximately 7 μg of pSMai135 digested with Pme I was added to 100 μl of protoplast solution and mixed gently, followed by 260 μl of PEG buffer, mixed, and incubated at room temperature for 30 minutes. STC (3 ml) was then added and mixed and the transformation solution was plated onto COVE plates using Aspergillus nidulans amdS selection. The plates were incubated at 28° C. for 5-7 days. Transformants were sub-cultured onto COVE2 plates and grown at 28° C.

Sixty-seven transformants designated SMA135 obtained with pSMai135 were subcultured onto fresh plates containing acetamide and allowed to sporulate for 7 days at 28° C.

The 67 SMA135 Trichoderma reesei transformants were cultivated in 125 ml baffled shake flasks containing 25 ml of cellulase-inducing media at pH 6.0 inoculated with spores of the transformants and incubated at 28° C. and 200 rpm for 7 days. Trichoderma reesei RutC30 was run as a control. Culture broth samples were removed at day 7. One ml of each culture broth was centrifuged at 15,700×g for 5 minutes in a micro-centrifuge and the supernatants transferred to new tubes. Samples were stored at 4° C. until enzyme assay. The supernatants were assayed for beta-glucosidase activity using p-nitrophenyl-beta-D-glucopyranoside as substrate, as described below.

Beta-glucosidase activity was determined at ambient temperature using 25 μl aliquots of culture supernatants, diluted 1:10 in 50 mM succinate pH 5.0, in 200 μl of 0.5 mg/ml p-nitrophenyl-beta-D-glucopyranoside as substrate in 50 mM succinate pH 5.0. After 15 minutes incubation the reaction was stopped by adding 100 μl of 1 M Tris-HCl pH 8.0 and the absorbance was read spectrophotometrically at 405 nm. One unit of beta-glucosidase activity corresponded to production of 1 μmol of p-nitrophenyl per minute per liter at pH 5.0, ambient temperature. Aspergillus niger beta-glucosidase (NOVOZYM™ 188, Novozymes A/S, Bagsværd, Denmark) was used as an enzyme standard.

A number of the SMA135 transformants showed beta-glucosidase activities several-fold higher than that secreted by Trichoderma reesei RutC30. One transformant designated SMA135-04 produced the highest beta-glucosidase activity.

SDS-PAGE was carried out using CRITERION® Tris-HCl (5% resolving) gels (Bio-Rad, Hercules, Calif., USA) with a CRITERION® System (Bio-Rad, Hercules, Calif., USA). Five μl of day 7 supernatants (see above) were suspended in 2× concentration of Laemmli Sample Buffer (Bio-Rad, Hercules, Calif., USA) and boiled in the presence of 5% beta-mercaptoethanol for 3 minutes. The supernatant samples were loaded onto a polyacrylamide gel and subjected to electrophoresis with 1× Tris/Glycine/SDS as running buffer (Bio-Rad, Hercules, Calif., USA). The resulting gel was stained with BIO-SAFE® Coomassie Blue Stain (Bio-Rad, Hercules, Calif., USA).

Of the 38 Trichoderma reesei SMA135 transformants analyzed by SDS-PAGE, 26 produced a protein of approximately 110 kDa that was not visible in Trichoderma reesei RutC30 as control. Transformant Trichoderma reesei SMA135-04 produced the highest level of beta-glucosidase as evidenced by abundance of the 110 kDa band seen by SDS-PAGE.

Trichoderma reesei SMA135-04 was spore-streaked through two rounds of growth on plates to insure it was a clonal strain, and multiple vials frozen prior to production scaled to process scale fermentor. The resulting protein broth was recovered from fungal cell mass, filtered, concentrated and formulated. The cellulolytic enzyme preparation was designated Cellulolytic Enzyme Composition #1.

### Example 13

### Construction of Expression Vector pSMai140

Expression vector pSMai140 was constructed by digesting plasmid pSATe111BG41 (WO 04/099228), which carries the Aspergillus oryzae beta-glucosidase variant BG41 full-length coding region (SEQ ID NO: 113 which encodes the amino acid sequence of SEQ ID NO: 114), with Nco I. The resulting 1243 bp fragment was isolated on a 1.0% agarose gel using TAE buffer and purified using a QIAQUICK® Gel Extraction Kit according to the manufacturer's instructions.

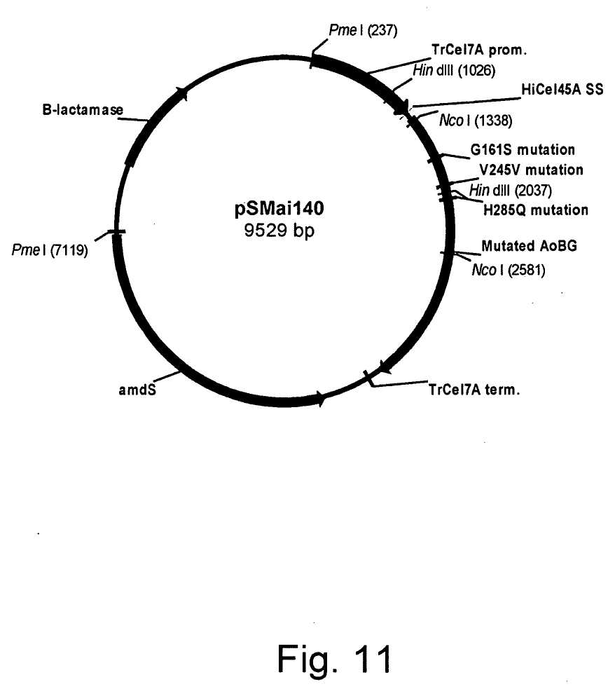

Expression vector pSMai135 was digested with Nco I and a 8286 bp fragment was isolated on a 1.0% agarose gel using TAE buffer and purified using a QIAQUICK® Gel Extraction Kit according to the manufacturer's instructions. The 1243 bp Nco I digested Aspergillus oryzae beta-glucosidase variant BG41 fragment was then ligated to the 8286 bp vector, using T4 DNA ligase (Roche, Indianapolis, Ind., USA) according to manufacturer's protocol, to create the expression vector pSMai140 (FIG. 11). Plasmid pSMai140 comprises the Trichoderma reesei cellobiohydrolase I (CEL7A) gene promoter and terminator operably linked to the Humicola insolens endoglucanase V signal sequence and the Aspergillus oryzae beta-glucosidase variant mature coding sequence.

### Example 14

### Transformation of Trichoderma reesei RutC30 with pSMai140

Plasmid pSMai140 was linearized with Pme I and transformed into the Trichoderma reesei RutC30 strain as described in Example 12. A total of 100 transformants were obtained from four independent transformation experiments, all of which were cultivated in shake flasks on cellulase-inducing medium, and the beta-glucosidase activity was measured from the culture medium of the transformants as described in Example 12. A number of Trichoderma reesei SMA140 transformants showed beta-glucosidase activities several fold higher than that of Trichoderma reesei RutC30.

The presence of the Aspergillus oryzae beta-glucosidase variant BG41 protein in the culture medium was detected by SDS-polyacrylamide gel electrophoresis as described in Example 12 and Coomassie staining from the same 13 culture supernatants from which enzyme activity were analyzed. All thirteen transformants that had high β-glucosidase activity, also expressed the approximately 110 KDa Aspergillus oryzae beta-glucosidase variant BG41, at varying yields.

The highest beta-glucosidase variant expressing transformant, as evaluated by beta-glucosidase activity assay and SDS-polyacrylamide gel electrophoresis, was designated Trichoderma reesei SMA140-43.

### Example 15

### Construction of Expression Vector pSaMe-F1

A DNA fragment containing 209 bp of the Trichoderma reesei cellobiohydrolase I gene promoter and the core region (nucleotides 1 to 702 of SEQ ID NO: 11, which encodes amino acids 1 to 234 of SEQ ID NO: 12; WO 91/17243) of the Humicola insolens endoglucanase V gene was PCR amplified using pMJ05 as template using the primers shown below.

Primer 995103:

(SEQ ID NO: 115)

5′-cccaagcttagccaagaaca-3′
Primer 995137:

(SEQ ID NO: 116)

5′-gggggaggaacgcatgggatctggacggc-3′

The amplification reactions (50 μl) were composed of 1× Pfx Amplification Buffer, 10 mM dNTPs, 50 mM MgSO₄, 10 ng/μl of pMJ05, 50 picomoles of 995103 primer, 50 picomoles of 995137 primer, and 2 units of Pfx DNA polymerase. The reactions were incubated in an EPPENDORF® MASTERCYCLER® 5333 programmed for 30 cycles each for 30 seconds at 94° C., 30 seconds at 55° C., and 60 seconds at 72° C. (3 minute final extension).

The reaction products were isolated on a 1.0% agarose gel using TAE buffer where a 911 bp product band was excised from the gel and purified using a QIAQUICK® Gel Extraction Kit according to the manufacturer's instructions.

A DNA fragment containing 806 bp of the Aspergillus oryzae beta-glucosidase variant BG41 gene was PCR amplified using pSMai140 as template and the primers shown below.

Primer 995133:

(SEQ ID NO: 117)

5′-gccgtccagatccccatgcgttcctccccc-3′
Primer 995111:

(SEQ ID NO: 118)

5′-ccaagcttgttcagagtttc-3′

The amplification reactions (50 μl) were composed of 1× Pfx Amplification Buffer, 10 mM dNTPs, 50 mM MgSO₄, 100 ng of pSMai140, 50 picomoles of 995133 primer, 50 picomoles of 995111 primer, and 2 units of Pfx DNA polymerase. The reactions were incubated in an EPPENDORF® MASTERCYCLER® 5333 programmed for 30 cycles each for 30 seconds at 94° C., 30 seconds at 55° C., and 120 seconds at 72° C. (3 minute final extension).

The reaction products were isolated by 1.0% agarose gel electrophoresis using TAE buffer where a 806 bp product band was excised from the gel and purified using a QIAQUICK® Gel Extraction Kit according to the manufacturer's instructions.

The two PCR fragments above were then subjected to overlapping PCR. The purified overlapping fragments were used as templates for amplification using primer 995103 (sense) and primer 995111 (antisense) described above to precisely fuse the 702 bp fragment comprising 209 bp of the Trichoderma reesei cellobiohydrolase I gene promoter and the Humicola insolens endoglucanase V core sequence to the 806 bp fragment comprising a portion of the Aspergillus oryzae beta-glucosidase variant BG41 coding region by overlapping PCR.

The amplification reactions (50 μl) were composed of 1× Pfx Amplification Buffer, 10 mM dNTPs, 50 mM MgSO₄, 2.5 μl of each fragment (20 ng/μl), 50 picomoles of 995103 primer, 50 picomoles of 995111 primer, and 2 units of Pfx DNA polymerase. The reactions were incubated in an EPPENDORF® MASTERCYCLER® 5333 programmed for an initial denaturation of 3 minutes at 95° C. followed by 30 cycles each for 1 minute of denaturation, 1 minute annealing at 60° C., and a 3 minute extension at 72° C.

The reaction products were isolated on a 1.0% agarose gel using TAE buffer where a 1.7 kb product band was excised from the gel and purified using a QIAQUICK® Gel Extraction Kit according to the manufacturer's instructions.

The 1.7 kb fragment was ligated into a pCR®4 Blunt Vector (Invitrogen, Carlsbad, Calif., USA) according to the manufacturer's instructions. The construct was then transformed into ONE SHOT® TOP10 Chemically Competent E. coli cells (Invitrogen, Carlsbad, Calif., USA) according to the manufacturer's rapid chemical transformation procedure. Colonies were selected and analyzed by plasmid isolation and digestion with Hind III to release the 1.7 kb overlapping PCR fragment.

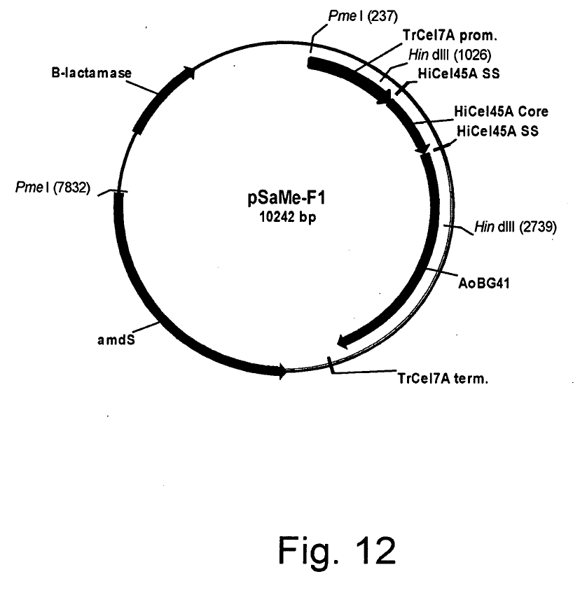

Plasmid pSMai140 was also digested with Hind III to linearize the plasmid. Both digested fragments were combined in a ligation reaction using a Rapid DNA Ligation Kit following the manufacturer's instructions to produce pSaMe-F1 (FIG. 12).

E. coli XL1-Blue Subcloning-Grade Competent Cells (Stratagene, La Jolla, Calif., USA) were transformed with the ligation product. Identity of the construct was confirmed by DNA sequencing of the Trichoderma reesei cellobiohydrolase I gene promoter, Humicola insolens endoglucanase V signal sequence, Humicola insolens endoglucanase V core, Humicola insolens endoglucanase V signal sequence, Aspergillus oryzae beta-glucosidase variant BG41, and the Trichoderma reesei cellobiohydrolase I gene terminator sequence from plasmids purified from transformed E. coli. One clone containing the recombinant plasmid was designated pSaMe-F1. Plasmid pSaMe-F1 comprises the Trichoderma reesei cellobiohydrolase I gene promoter and terminator and the Humicola insolens endoglucanase V signal peptide sequence linked directly to the Humicola insolens endoglucanase V core polypeptide which are fused directly to the Humicola insolens endoglucanase V signal peptide which is linked directly to the Aspergillus oryzae beta-glucosidase variant BG41 mature coding sequence. The DNA sequence and deduced amino acid sequence of the Aspergillus oryzae beta-glucosidase variant BG fusion protein is shown in SEQ ID NOs: 57 and 58, respectively.

### Example 16

### Transformation of Trichoderma reesei RutC30 with pSaMe-F1

Shake flasks containing 25 ml of YP medium supplemented with 2% glucose and 10 mM uridine were inoculated with 5×10⁷ spores of Trichoderma reesei RutC30. Following incubation overnight for approximately 16 hours at 27° C., 90 rpm, the mycelia were collected using a Vacuum Driven Disposable Filtration System. The mycelia were washed twice in 100 ml of deionized water and twice in 1.2 M sorbitol. Protoplasts were generated as described in Example 12.

Two micrograms of pSaMe-F1 DNA linearized with Pme I, 100 μl of Trichoderma reesei RutC30 protoplasts, and 50% PEG (4000) were mixed and incubated for 30 minutes at room temperature. Then 3 ml of STC were added and the contents were poured onto a COVE plate supplemented with 10 mM uridine. The plate was then incubated at 28° C. Transformants began to appear by day 6 and were picked to COVE2 plates for growth at 28° C. and 6 days. Twenty-two Trichoderma reesei transformants were recovered.

Transformants were cultivated in shake flasks on cellulase-inducing medium and beta-glucosidase activity was measured as described in Example 12. A number of pSaMe-F1 transformants produced beta-glucosidase activity. One transformant, designated Trichoderma reesei SaMeF1-9, produced the highest amount of beta-glucosidase, and had twice the activity of a strain expressing the Aspergillus oryzae beta-glucosidase variant (Example 15).

Endoglucanase activity was assayed using a carboxymethyl cellulose (CMC) overlay assay according to Beguin, 1983, Analytical Biochem. 131(2): 333-336. Five μg of total protein from five of the broth samples (those having the highest beta-glucosidase activity) were diluted in Native Sample Buffer (Bio-Rad, Hercules, Calif., USA) and run on a CRITERION® 8-16% Tris-HCl gel using 10× Tris/glycine running buffer (Bio-Rad, Hercules, Calif., USA) and then the gel was laid on top of a plate containing 1% carboxymethylcellulose (CMC). After 1 hour incubation at 37° C., the gel was stained with 0.1% Congo Red for 20 minutes. The plate was then destained using 1 M NaCl in order to identify regions of clearing indicative of endoglucanase activity. Two clearing zones were visible, one upper zone around 110 kDa and a lower zone around 25 kDa. The predicted protein size of the Humicola insolens endoglucanase V and Aspergillus oryzae beta-glucosidase variant BG41 fusion is 118 kDa if the two proteins are not cleaved and remain as a single polypeptide; glycosylation of the individual endoglucanase V core domain and of the beta-glucosidase leads to migration of the individual proteins at higher mw than predicted from the primary sequence. If the two proteins are cleaved then the predicted sizes for the Humicola insolens endoglucanase V core domain is 24 kDa and 94 kDa for Aspergillus oryzae beta-glucosidase variant BG41. Since there was a clearing zone at 110 kDa this result indicated that minimally a population of the endoglucanase and beta-glucosidase fusion protein remains intact as a single large protein. The lower clearing zone most likely represents the endogenous endoglucanase activity, and possibly additionally results from partial cleavage of the Humicola insolens endoglucanase V core domain from the Aspergillus oryzae β-glucosidase.

The results demonstrated the Humicola insolens endoglucanase V core was active even though it was linked to the Aspergillus oryzae beta-glucosidase. In addition, the increase in beta-glucosidase activity appeared to result from increased secretion of protein relative to the secretion efficiency of the non-fusion beta-glucosidase. By linking the Aspergillus oryzae beta-glucosidase variant BG41 sequence to the efficiently secreted Humicola insolens endoglucanase V core, more beta-glucosidase was secreted.

### Example 17

### Construction of Vector pSaMe-FX

Plasmid pSaMe-FX was constructed by modifying pSaMe-F1. Plasmid pSaMe-F1 was digested with Bst Z17 and Eco RI to generate a 1 kb fragment that contained the beta-glucosidase variant BG41 coding sequence and a 9.2 kb fragment containing the remainder of the plasmid. The fragments were separated on a 1.0% agarose gel using TAE buffer and the 9.2 kb fragment was excised and purified using a QIAQUICK® Gel Extraction Kit according to the manufacturer's instructions. Plasmid pSMai135 was also digested with Bst Z17 and Eco RI to generate a 1 kb fragment containing bases homologous to the Aspergillus oryzae beta-glucosidase variant BG41 coding sequence and a 8.5 kb fragment containing the remainder of the plasmid. The 1 kb fragment was isolated and purified as above.

The 9.2 kb and 1 kb fragments were combined in a ligation reaction using a Rapid DNA Ligation Kit following the manufacturer's instructions to produce pSaMe-FX, which is identical to pSaMe-F1 except that it contained the wild-type beta-glucosidase mature coding sequence rather than the variant mature coding sequence.

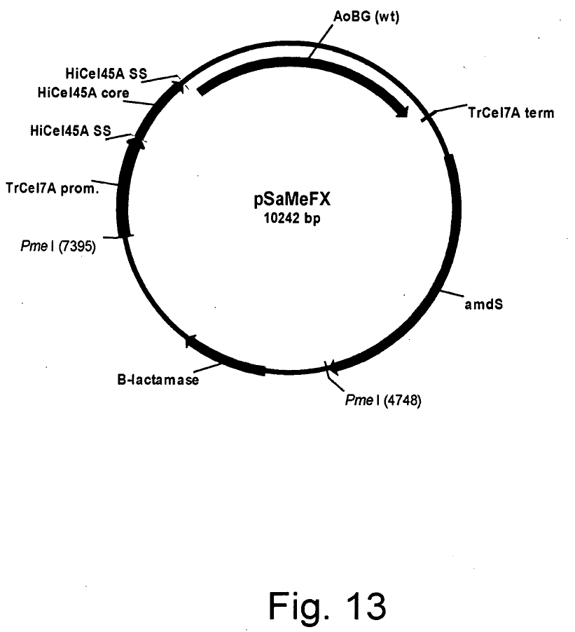

E. coli SURE® Competent Cells (Stratagene, La Jolla, Calif., USA) were transformed with the ligation product. Identity of the construct was confirmed by DNA sequencing of the Trichoderma reesei cellobiohydrolase I gene promoter, Humicola insolens endoglucanase V signal sequence, Humicola insolens endoglucanase V core sequence, Humicola insolens endoglucanase V signal sequence, Aspergillus oryzae beta-glucosidase mature coding sequence, and the Trichoderma reesei cellobiohydrolase I gene terminator sequence from plasmids purified from transformed E. coli. One clone containing the recombinant plasmid was designated pSaMe-FX (FIG. 13). The DNA sequence and deduced amino acid sequence of the Aspergillus oryzae beta-glucosidase fusion protein is shown in SEQ ID NOs: 59 and 60, respectively.

### Example 18

### Transformation and Expression of Trichoderma Transformants

The pSaMe-FX construct was linearized with Pme I and transformed into the Trichoderma reesei RutC30 strain as described in Example 16. A total of 63 transformants were obtained from a single transformation. Transformants were cultivated in shake flasks on cellulase-inducing medium, and beta-glucosidase activity was measured as described in Example 12. A number of pSaMe-FX transformants produced beta-glucosidase activity. One transformant designated SaMe-FX16 produced twice the amount of beta-glucosidase activity compared to Trichoderma reesei SaMeF1-9 (Example 16).

### Example 19

### Analysis of Trichoderma reesei Transformants

A fusion protein was constructed as described in Example 15 by fusing the Humicola insolens endoglucanase V core (containing its own native signal sequence) with the Aspergillus oryzae beta-glucosidase variant BG41 mature coding sequence linked to the Humicola insolens endoglucanase V signal sequence. This fusion construct resulted in a two-fold increase in secreted beta-glucosidase activity compared to the Aspergillus oryzae beta-glucosidase variant BG41 mature coding sequence linked to the Humicola insolens endoglucanase V signal sequence. A second fusion construct was made as described in Example 17 consisting of the Humicola insolens endoglucanase V core (containing its own signal sequence) fused with the Aspergillus oryzae wild-type beta-glucosidase coding sequence linked to the Humicola insolens endoglucanase V signal sequence, and this led to an even further improvement in beta-glucosidase activity. The strain transformed with the wild-type fusion had twice the secreted beta-glucosidase activity relative to the strain transformed with the beta-glucosidase variant BG41 fusion.

### Example 20

### Cloning of the Beta-Glucosidase Fusion Protein Encoding Sequence into an Aspergillus oryzae Expression Vector

Two synthetic oligonucleotide primers, shown below, were designed to PCR amplify the full-length open reading frame from pSaMeFX encoding the beta-glucosidase fusion protein.

PCR Forward primer:

(SEQ ID NO: 119)

5′-GGACTGCGCAGCATGCGTTC-3′
PCR Reverse primer:

(SEQ ID NO: 120)

5′-AGTTAATTAATTACTGGGCCTTAGGCAGCG-3′

Bold letters represent coding sequence. The underlined “G” in the forward primer represents a base change introduced to create an Sph I restriction site. The remaining sequence contains sequence identity compared with the insertion sites of pSaMeFX. The underlined sequence in the reverse primer represents a Pac I restriction site added to facilitate the cloning of this gene in the expression vector pAlLo2 (WO 04/099228).

Fifty picomoles of each of the primers above were used in a PCR reaction containing 50 ng of pSaMeFX DNA, 1× Pfx Amplification Buffer, 6 μl of 10 mM blend of dATP, DTTP, dGTP, and dCTP, 2.5 units of PLATINUM® Pfx DNA Polymerase, and 1 μl of 50 mM MgSO₄ in a final volume of 50 μl. The amplification reaction was incubated in an EPPENDORF® MASTERCYCLER® 5333 programmed for 1 cycle at 98° C. for 2 minutes; and 35 cycles each at 96° C. for 30 seconds, 61° C. for 30 seconds, and 68° C. for 3 minutes. After the 35 cycles, the reaction was incubated at 68° C. for 10 minutes and then cooled at 10° C. A 3.3 kb PCR reaction product was isolated on a 0.8% GTG®-agarose gel using TAE buffer and 0.1 μg of ethidium bromide per ml. The DNA was visualized with the aid of a DARK READERM to avoid UV-induced mutations. A 3.3 kb DNA band was excised with a disposable razor blade and purified with an ULTRAFREE®-DA spin cup according to the manufacturer's instructions.

The purified 3.3 kb PCR product was cloned into a pCR®4Blunt-TOPO® vector (Invitrogen, Carlsbad, Calif., USA). Four microliters of the purified PCR product were mixed with 1 μl of a 2 M sodium chloride solution and 1 μl of the TOPO® vector. The reaction was incubated at room temperature for 15 minutes and then 2 μl of the reaction were used to transform ONE SHOT® TOP10 Chemically Competent E. coli cells according to the manufacturer's instructions. Three aliquots of 83 μl each of the transformation reaction were spread onto three 150 mm 2×YT plates supplemented with 100 μg of ampicillin per ml and incubated overnight at 37° C.

Eight recombinant colonies were used to inoculate liquid cultures containing 3 ml of LB medium-supplemented with 100 μg of ampicillin per ml. Plasmid DNA was prepared from these cultures using a BIOROBOT® 9600. Clones were analyzed by restriction enzyme digestion with Pac I. Plasmid DNA from each clone was digested with Pac I and analyzed by 1.0% agarose gel electrophoresis using TAE buffer. All eight clones had the expected restriction digest pattern and clones 5, 6, 7, and 8 were selected to be sequenced to confirm that there were no mutations in the cloned insert. Sequence analysis of their 5′ and 3′ ends indicated that all 4 clones had the correct sequence. Clones 5 and 7 were selected for further sequencing. Both clones were sequenced to Phred Q values of greater than 40 to ensure that there were no PCR induced errors. Clones 5 and 7 were shown to have the expected sequence and clone 5 was selected for re-cloning into pAlLo2.

Plasmid DNA from clone 5 was linearized by digestion with Sph I. The linearized clone was then blunt-ended by adding 1.2 μl of a 10 mM blend of dATP, dTTP, dGTP, and dCTP and 6 units of T4 DNA polymerase (New England Bioloabs, Inc., Ipswich, Mass., USA). The mixture was incubated at 12° C. for 20 minutes and then the reaction was stopped by adding 1 μl of 0.5 M EDTA and heating at 75° C. for 20 minutes to inactivate the enzyme. A 3.3 kb fragment encoding the beta-glucosidase fusion protein was purified by gel electrophoresis and ultrafiltration as described above.

The vector pAlLo2 was linearized by digestion with Nco I. The linearized vector was then blunt-ended by adding 0.5 μl of a 10 mM blend of dATP, dTTP, dGTP, and dCTP and one unit of DNA polymerase I. The mixture was incubated at 25° C. for 15 minutes and then the reaction was stopped by adding 1 μl of 0.5M EDTA and heating at 75° C. for 15 minutes to inactivate the enzymes. Then the vector was digested with Pac I. The blunt-ended vector was purified by gel electrophoresis and ultrafiltration as described above.

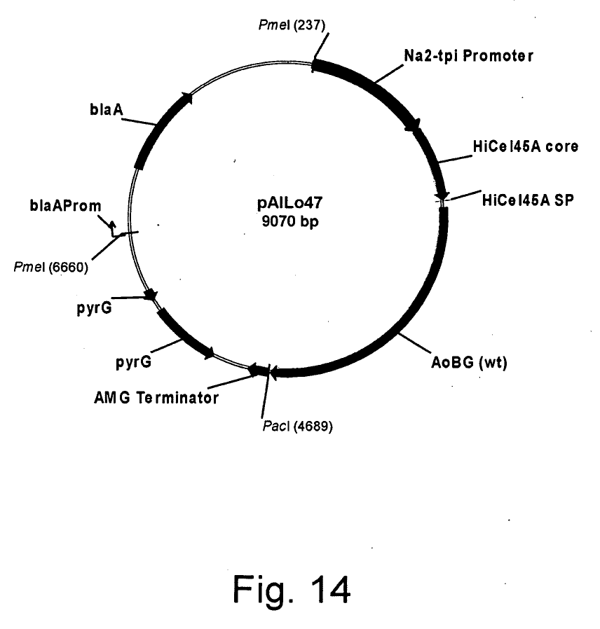

Cloning of the 3.3 kb fragment encoding the beta-glucosidase fusion protein into the linearized and purified pAlLo2 vector was performed with a Rapid DNA Ligation Kit. A 1 μl sample of the reaction was used to transform E. coli XL10 SOLOPACK® Gold cells (Stratagene, La Jolla, Calif., USA) according to the manufacturer's instructions. After the recovery period, two 100 μl aliquots from the transformation reaction were plated onto two 150 mm 2×YT plates supplemented with 100 μg of ampicillin per ml and incubated overnight at 37° C. A set of eight putative recombinant clones was selected at random from the selection plates and plasmid DNA was prepared from each one using a BIOROBOT® 9600. Clones 1-4 were selected for sequencing with pAlLo2-specific primers to confirm that the junction vector/insert had the correct sequence. Clone 3 had a perfect vector/insert junction and was designated pAILo47 (FIG. 14).

In order to create a marker-free expression strain, a restriction endonuclease digestion was performed to separate the blaA gene that confers resistance to the antibiotic ampicillin from the rest of the expression construct. Thirty micrograms of pAILo47 were digested with Pme I. The digested DNA was then purified by agarose gel electrophoresis as described above. A 6.4 kb DNA band containing the expression construct but lacking the blaA gene was excised with a razor blade and purified with a QIAQUICK® Gel Extraction Kit.

### Example 21

### Expression of the Humicola insolens/Aspergillus oryzae cel45Acore-cel3A Fusion Gene in Aspergillus oryzae JaL355

Aspergillus oryzae JaL355 (WO 00/240694) protoplasts were prepared according to the method of Christensen et al., 1988, supra. Ten microliters of the purified expression construct of Example 20 were used to transform Aspergillus oryzae JaL355 protoplasts. The transformation of Aspergillus oryzae JaL355 yielded approximately 90 transformants. Fifty transformants were isolated to individual PDA plates and incubated for five days at 34° C.

Forty-eight confluent spore plates were washed with 3 ml of 0.01% TWEEN® 80 and the spore suspension was used to inoculate 25 ml of MDU2BP medium in 125 ml glass shake flasks. Transformant cultures were incubated at 34° C. with constant shaking at 200 rpm. After 5 days, 1 ml aliquots of each culture was centrifuged at 12,000×g and their supernatants collected. Five μl of each supernatant were mixed with an equal volume of 2× loading buffer (10% beta-mercaptoethanol) and loaded onto a 1.5 mm 8%-16% Tris-Glycine SDS-PAGE gel and stained with BIO-SAFE® Coomassie Blue Stain. SDS-PAGE profiles of the culture broths showed that 33 out of 48 transformants were capable of expressing a new protein with an apparent molecular weight very close to the expected 118 kDa. Transformant 21 produced the best yield and was selected for further studies.

### Example 22

### Single Spore Isolation of Aspergillus oryzae JaL355 Transformant 21

Aspergillus oryzae JaL355 transformant 21 spores were spread onto a PDA plate and incubated for five days at 34° C. A small area of the confluent spore plate was washed with 0.5 ml of 0.01% TWEEN® 80 to resuspend the spores. A 100 μl aliquot of the spore suspension was diluted to a final volume of 5 ml with 0.01% TWEEN® 80. With the aid of a hemocytometer the spore concentration was determined and diluted to a final concentration of 0.1 spores per microliter. A 200 μl aliquot of the spore dilution was spread onto 150 mm Minimal medium plates and incubated for 2-3 days at 34° C. Emerging colonies were excised from the plates and transferred to PDA plates and incubated for 3 days at 34° C. Then the spores were spread across the plates and incubated again for 5 days at 34° C.

The confluent spore plates were washed with 3 ml of 0.01% TWEEN® 80 and the spore suspension was used to inoculate 25 ml of MDU2BP medium in 125 ml glass shake flasks. Single-spore cultures were incubated at 34° C. with constant shaking at 200 rpm. After 5 days, a 1 ml aliquot of each culture was centrifuged at 12,000×g and their supernatants collected. Five μl of each supernatant were mixed with an equal volume of 2× loading buffer (10% beta-mercaptoethanol) and loaded onto a 1.5 mm 8%-16% Tris-Glycine SDS-PAGE gel and stained with BIO-SAFE® Commassie Blue Stain. SDS-PAGE profiles of the culture broths showed that all eight transformants were capable of expressing the beta-glucosidase fusion protein at very high levels and one of cultures designated Aspergillus oryzae JaL355AILo47 produced the best yield.

### Example 23

### Construction of pCW087

Two synthetic oligonucleotide primers shown below were designed to PCR amplify a Thermoascus aurantiacus GH61A polypeptide gene from plasmid pDZA2-7 (WO 2005/074656). The forward primer results in a blunt 5′ end and the reverse primer incorporates a Pac I site at the 3′ end.

| | |
|---|---|
| Forward Primer: | |
| 5′-ATGTCCTTTTCCAAGATAATTGCTACTG-3′ | (SEQ ID NO: 121) |
| Reverse Primer: | |
| 5′-GCTTAATTAACCAGTATACAGAGGAG-3′ | (SEQ ID NO: 122) |

Fifty picomoles of each of the primers above were used in a PCR reaction consisting of 50 ng of pDZA2-7, 1 μl of 10 mM blend of dATP, dTTP, dGTP, and dCTP, 5 μl of 10× ACCUTAQ™ DNA Polymerase Buffer (Sigma-Aldrich, St. Louis, Mo., USA), and 5 units of ACCUTAQ™ DNA Polymerase (Sigma-Aldrich, St. Louis, Mo., USA), in a final volume of 50 PI. An EPPENDORF® MASTERCYCLER® 5333 was used to amplify the DNA fragment programmed for 1 cycle at 95° C. for 3 minutes; 30 cycles each at 94° C. for 45 seconds, 55° C. for 60 seconds, and 72° C. for 1 minute 30 seconds. After the 25 cycles, the reaction was incubated at 72° C. for 10 minutes and then cooled at 4° C. until further processing. The 3′ end of the Thermoascus aurantiacus GH61A PCR fragment was digested using Pac I. The digestion product was purified using a MINELUTE™ Reaction Cleanup Kit (QIAGEN Inc., Valencia, Calif., USA) according to the manufacturer's instructions.

The GH61A fragment was directly cloned into pSMai155 (WO 2005/074647) utilizing a blunted Nco I site at the 5′ end and a Pac I site at the 3′ end. Plasmid pSMai155 was digested with Nco I and Pac I. The Nco I site was then rendered blunt using Klenow enzymes to fill in the 5′ recessed Nco I site. The Klenow reaction consisted of 20 μl of the pSMai155 digestion reaction mix plus 1 mM dNTPs and 1 μl of Klenow enzyme, which was incubated briefly at room temperature. The newly linearized pSMai155 plasmid was purified using a MINELUTE™ Reaction Cleanup Kit according to the manufacturer's instructions. These reactions resulted in the creation a 5′ blunt end and 3′ Pac I site compatible to the newly generated GH61A fragment. The GH61A fragment was then cloned into pSMai155 expression vector using a Rapid DNA Ligation Kit following the manufacturer's instructions. E. coli XL1-Blue Subcloning-Grade Competent Cells (Stratagene, La Jolla, Calif., USA) were transformed with the ligation product. Identity of the construct was confirmed by DNA sequencing of the GH61A coding sequence from plasmids purified from transformed E. coli. One E. coli clone containing the recombinant plasmid was designated pCW087-8.

### Example 24

### Construction of pSaMe-Ta61A

Expression vector pSaMe-Ta61 was constructed by digesting plasmid pMJ09, which harbors the amdS selectable marker, with Nsi I, which liberated a 2.7 kb amdS fragment. The 2.7 kb amdS fragment was then isolated by 1.0% agarose gel electrophoresis using TAE buffer and purified using a QIAQUICK® Gel Extraction Kit.

Expression vector pCW087 was digested with Nsi I and a 4.7 kb fragment was isolated by 1.0% agarose gel electrophoresis using TAE buffer and purified using a QIAQUICK® Gel Extraction Kit. The 2.7 kb amdS fragment was then ligated to the 4.7 kb vector fragment, using T4 DNA ligase (Roche, Indianapolis, Ind., USA) according to manufacturer's protocol, to create the expression vector pSaMe-Ta61A. Plasmid pSaMe-Ta61A comprises the Trichoderma reesei cellobiohydrolase I (CEL7A) gene promoter and terminator operably linked to the Thermoascus aurantiacus GH61A mature coding sequence.

### Example 25

### Construction of Trichoderma reesei Strain SaMe-MF268

A co-transformation was utilized to introduce plasmids pSaMe-FX and pSaMe-Ta61A into Trichoderma reesei RutC30. Plasmids pSaMe-FX and pSaMe-Ta61A were introduced into Trichoderma reesei RutC30 by PEG-mediated transformation (Penttila et al., 1987, supra). Each plasmid contained the Aspergillus nidulans amdS gene to enable transformants to grow on acetamide as the sole nitrogen source.

Trichoderma reesei RutC30 was cultivated at 27° C. and 90 rpm in 25 ml of YP medium supplemented with 2% (w/v) glucose and 10 mM uridine for 17 hours. Mycelia were collected by filtration using a Vacuum Driven Disposable Filtration System and washed twice with deionized water and twice with 1.2 M sorbitol. Protoplasts were generated by suspending the washed mycelia in 20 ml of 1.2 M sorbitol containing 15 mg of GLUCANEX® per ml and 0.36 units of chitinase (Sigma Chemical Co., St. Louis, Mo., USA) per ml and incubating for 15-25 minutes at 34° C. with gentle shaking at 90 rpm. Protoplasts were collected by centrifuging for 7 minutes at 400×g and washed twice with cold 1.2 M sorbitol. The protoplasts were counted using a haemacytometer and re-suspended in STC to a final concentration of 1×10⁸ protoplasts per ml. Excess protoplasts were stored in a Cryo 1° C. Freezing Container at −80° C.

Approximately 4 μg each of plasmids pSaMe-FX and pSaMe-Ta61A were digested with Pme I to facilitate removal of the ampicillin resistance marker. Following digestion with Pme I the linear fragments were purified by 1% agarose gel electrophoresis using TAE buffer. A 7.5 kb fragment from pSaMe-FX and a 4.7 kb fragment from pSaMe-Ta61A were excised from the gel and purified using a QIAQUICK® Gel Extraction Kit according to the manufacturer's instructions. These purified fragments contain the amdS selectable marker cassette and the Trichoderma reesei cbh1 gene promoter and terminator. Additionally, the fragment includes the Humicola insolens EGV core/Aspergillus oryzae BG fusion coding sequence or the Thermoascus aurentiacus GH61A coding sequence. The fragments used in transformation did not contain antibiotic resistance markers, as the ampR fragment was removed by this gel purification step. The purified fragments were then added to 100 μl of protoplast solution and mixed gently, followed by 260 μl of PEG buffer, mixed, and incubated at room temperature for 30 minutes. STC (3 ml) was then added and mixed and the transformation solution was plated onto COVE plates using Aspergillus nidulans amdS selection. The plates were incubated at 28° C. for 5-7 days. Transformants were sub-cultured onto COVE2 plates and grown at 28° C.

Over 400 transformants were subcultured onto fresh plates containing acetamide and allowed to sporulate for 7 days at 28° C.

The Trichoderma reesei transformants were cultivated in 125 ml baffled shake flasks containing 25 ml of cellulase-inducing medium at pH 6.0 inoculated with spores of the transformants and incubated at 28° C. and 200 rpm for 5 days. Trichoderma reesei RutC30 was run as a control. Culture broth samples were removed at day 5. One ml of each culture broth was centrifuged at 15,700×g for 5 minutes in a micro-centrifuge and the supernatants transferred to new tubes.

SDS-PAGE was carried out using CRITERION® Tris-HCl (5% resolving) gels with a CRITERION® System. Five μl of day 5 supernatants (see above) were suspended in 2× concentration of Laemmli Sample Buffer (Bio-Rad, Hercules, Calif., USA) and boiled in the presence of 5% beta-mercaptoethanol for 3 minutes. The supernatant samples were loaded onto a polyacrylamide gel and subjected to electrophoresis with 1× Tris/Glycine/SDS as running buffer (Bio-Rad, Hercules, Calif., USA). The resulting gel was stained with BIO-SAFE® Coomassie Blue Stain. Transformants showing expression of both the Thermoascus aurantiacus GH61A polypeptide and the fusion protein consisting of the Humicola insolens endoglucanase V core (CEL45A) fused with the Aspergillus oryzae beta-glucosidase as seen by visualization of bands on SDS-PAGE gels were then tested in PCS hydrolysis reactions to identify the strains producing the best hydrolytic broths.

### Example 26

### Identification of Trichoderma reesei Strain SaMe-MF268

The transformants showing expression of both the Thermoascus aurantiacus GH61A polypeptide and the Aspergillus oryzae beta-glucosidase fusion protein were cultivated in 125 ml baffled shake flasks containing 25 ml of cellulase-inducing media at pH 6.0 inoculated with spores of the transformants and incubated at 28° C. and 200 rpm for 5 days.

The shake flask culture broths were centrifuged at 6000×g and filtered using a STERICUP™ EXPRESS™ (Millipore, Bedford, Mass., USA) to 0.22 μm prior to hydrolysis. The activities of the culture broths were measured by their ability to hydrolyze the PCS and produce sugars detectable by a chemical assay of their reducing ends.

Corn stover was pretreated at the U.S. Department of Energy National Renewable Energy Laboratory (NREL), Boulder, Colo., USA, using dilute sulfuric acid. The following conditions were used for the pretreatment: 0.048 g sulfuric acid/9 dry biomass at 190° C. and 25% w/w dry solids for around 1 minute. The water-insoluble solids in the pretreated corn stover (PCS) contained 59.2% cellulose as determined by a limit digest of PCS to release glucose and cellobiose. Prior to enzymatic hydrolysis, the PCS was washed with a large volume of double deionized water; the dry weight of the water-washed PCS was found to be 17.73%.

PCS in the amount of 1 kg was suspended in approximately 20 liters of double deionized water and, after the PCS settled, the water was decanted. This was repeated until the wash water was above pH 4.0, at which time the reducing sugars were lower than 0.06 g per liter. For small volume assays (e.g., 1 ml) the settled slurry was sieved through 100 Mesh screens to ensure ability to pipette. Percent dry weight content of the washed PCS was determined by drying the sample at a 105° C. oven for at least 24 hours (until constant weight) and comparing to the wet weight.

PCS hydrolysis was performed in a 1 ml volume in 96-deep-well plates (Axygen Scientific) heat sealed by an ALPS 300™ automated lab plate sealer (ABgene Inc., Rochester, N.Y., USA). PCS concentration was 10 g per liter in 50 mM sodium acetate pH 5.0. PCS hydrolysis was performed at 50° C. without additional stirring except as during sampling as described. Each reaction was performed in triplicate. Released reducing sugars were analyzed by p-hydroxy benzoic acid hydrazide (PHBAH) reagent as described below.

A volume of 0.8 ml of PCS (12.5 g per liter in water) was pipetted into each well of 96-deep-well plates, followed by 0.10 ml of 0.5 M sodium acetate pH 5.0, and then 0.10 ml of diluted enzyme solution to start the reaction with a final reaction volume of 1.0 ml and PCS concentration of 10 g per liter. Plates were sealed. The reaction mixture was mixed by inverting the deep-well plate at the beginning of hydrolysis and before taking each sample time point. At each sample time point the plate was mixed and then the deep-well plate was centrifuged (Sorvall RT7 with RTH-250 rotor) at 2000 rpm for 10 minutes before 20 μl of hydrolysate (supernatant) was removed and added to 180 μl of 0.4% NaOH in a 96-well microplate. This stopped solution was further diluted into the proper range of reducing sugars, when necessary. The reducing sugars released were assayed by para-hydroxy benzoic acid hydrazide reagent (PHBAH, 4-hydroxy benzyhydrazide, Sigma Chemical Co., St. Louis, Mo., USA): 50 μl of PHBAH reagent (1.5%) was mixed with 100 μl of sample in a V-bottom 96-well THERMOWELL™ plate (Costar 6511), incubated on a plate heating block at 95° C. for 10 minutes, then 50 μl of double deionized water was added to each well, mixed and 100 μl was transferred to another flat-bottom 96-well plate (Costar 9017) and absorbance read at 410 nm. Reducing sugar was calculated using a glucose calibration curve under the same conditions. Percent conversion of cellulose to reducing sugars was calculated as:

% conversion=reducing sugars(mg/ml)/(cellulose added(mg/ml)×1.11)

The factor 1.11 corrects for the weight gain in hydrolyzing cellulose to glucose.

Following the 1 ml PCS hydrolysis testing, the top candidates were grown in duplicate in 2 liter fermentors.

Shake flask medium was composed per liter of 20 g of dextrose, 10 g of corn steep solids, 1.45 g of (NH₄)₂SO₄, 2.08 g of KH₂PO₄, 0.36 g of CaCl₂, 0.42 g of MgSO₄.7H₂O, and 0.42 ml of trace metals solution. Trace metals solution was composed per liter of 216 g of FeCl₃.6H₂O, 58 g of ZnSO₄.7H₂O, 27 g of MnSO₄.H₂O, 10 g of CuSO₄.5H₂O, 2.4 g of H₃BO₃, and 336 g of citric acid:

Ten ml of shake flask medium was added to a 500 ml shake flask. The shake flask was inoculated with two plugs from a solid plate culture and incubated at 28° C. on an orbital shaker at 200 rpm for 48 hours. Fifty ml of the shake flask broth was used to inoculate a 3 liter fermentation vessel.

Fermentation batch medium was composed per liter of 30 g of cellulose, 4 g of dextrose, 10 g of corn steep solids, 3.8 g of (NH₄)₂SO₄, 2.8 g of KH₂PO₄, 2.64 g of CaCl₂, 1.63 g of MgSO₄.7H₂O, 1.8 ml of anti-foam, and 0.66 ml of trace metals solution. Trace metals solution was composed per liter of 216 g of FeCl₃.6H₂O, 58 g of ZnSO₄.7H₂O, 27 g of MnSO₄.H₂O, 10 g of CuSO₄.5H₂O, 2.4 g of H₃BO₃, and 336 g of citric acid. Fermentation feed medium was composed of dextrose and cellulose.

A total of 1.8 liters of the fermentation batch medium was added to a 3 liter fermentor. Fermentation feed medium was dosed at a rate of 0 to 4 g/l/hr for a period of 165 hours. The fermentation vessel was maintained at a temperature of 28° C. and pH was controlled to a set-point of 4.75+/−0.1. Air was added to the vessel at a rate of 1 vvm and the broth was agitated by Rushton impeller rotating at 1100 to 1300 rpm. At the end of the fermentation, whole broth was harvested from the vessel and centrifuged at 3000 rpm×g to remove the biomass. The supernatant was sterile filtered and stored at 35 to 40° C.

Total protein concentration was determined and broths were re-tested in 50 g PCS hydrolysis reactions as described below. Enzyme dilutions were prepared fresh before each experiment from stock enzyme solutions, which were stored at 4° C.

Hydrolysis of PCS was conducted using 125 ml screw-top Erlenmeyer flasks (VWR, West Chester, Pa., USA) using a total reaction mass of 50 g according to NREL Laboratory Analytical Protocol #008. In this protocol hydrolysis of PCS (approximately 11.4% in PCS and 6.8% cellulose in aqueous 50 mM sodium acetate pH 5.0) was performed using different protein loadings (expressed as mg of protein per gram of cellulose) of the 2 liter fermentation broth sample. Testing of PCS hydrolyzing capability was performed at 50° C. with orbital shaking at 150 rpm using an INNOVA® 4080 Incubator (New Brunswick Scientific, Edison, N.J., USA). Aliquots were taken during the course of hydrolysis at 72, 120, and 168 hours and centrifuged, and the supernatant liquid was filtered using a MULTISCREEN® HV 0.45 μm membrane (Millipore, Billerica, Mass., USA) by centrifugation at 2000 rpm for 10 minutes using a SORVALL® RT7 plate centrifuge (Thermo Fisher Scientific, Waltham, Mass., USA). When not used immediately, filtered aliquots were frozen at −20° C. Sugar concentrations of samples diluted in 0.005 M H₂SO₄ were measured after elution by 0.005 M H₂SO₄ at a flow rate of 0.4 ml per minute from a 4.6×250 mm AMINEX® HPX-87H column (Bio-Rad, Hercules, Calif., USA) at 65° C. with quantitation by integration of glucose and cellobiose signal from refractive index detection using a CHEMSTATION® AGILENT® 1100 HPLC (Agilent Technologies, Santa Clara, Calif., USA) calibrated by pure sugar samples. The resultant equivalents were used to calculate the percentage of cellulose conversion for each reaction.

The degree of cellulose conversion to glucose plus cellobiose sugars (conversion, %) was calculated using the following equation:

Conversion_{(%)}=(glucose+cellobiose×1.053)_{(mg/ml)}×100×162/(cellulose_{(mg/ml)}×180)=(glucose+cellobiose×1.053)_{(mg/ml)}×100/(cellulose_{(mg/ml)}×1.111)

In this equation the factor 1.111 reflects the weight gain in converting cellulose to glucose, and the factor 1.053 reflects the weight gain in converting cellobiose to glucose.

The results of the PCS hydrolysis reactions in the 50 g flask assay described above are shown in Table 2. One strain that produced the highest performing broth was designated Trichoderma reesei SaMe-MF268.

TABLE 2
Percent conversion to sugars at 168 hour timepoint

| | Percent conversion (glucose plus cellobiose) for | |
|---|---|---|
| | protein loading | |
| Broth ID-Strain Name | 2.5 mg/g cellulose | 4.0 mg/g cellulose |
| XCL-461-SaMe- | 66.29 | 80.08 |
| MF268 | | |
| XCL-465-SaMe- | 69.13 | 82.80 |
| MF268 | | |
| XCL-462-SaMe- | 62.98 | 77.99 |
| MF330 | | |
| XCL-466-SaMe- | 63.34 | 77.90 |
| MF330 | | |
| XCL-463-SaMe- | 64.03 | 78.45 |
| MF377 | | |
| XCL-467-SaMe- | 64.19 | 79.06 |
| MF377 | | |

### Example 27

### Construction of Vector pSaMe-FH

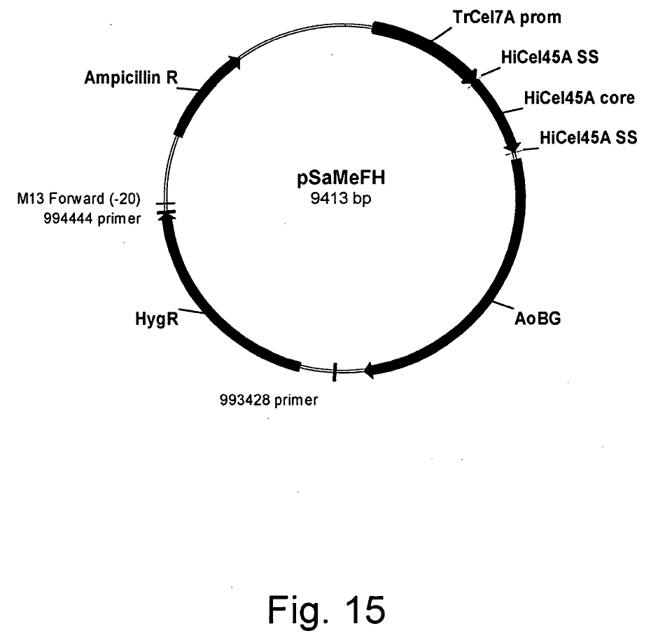

Expression vector pSaMe-FH (FIG. 15) was constructed by digesting plasmid pSMai155 (WO 2005/074647) and plasmid pSaMe-FX (Example 17) with Bsp 1201 and Pac I. The 5.5 kb fragment from pSMai155 and the 3.9 kb fragment from pSaMeFX were isolated by 1.0% agarose gel electrophoresis using TAE buffer and purified using a QIAQUICK® Gel Extraction Kit. The two fragments were then ligated using T4 DNA ligase according to manufacturer's protocol. E. coli SURE® Competent Cells were transformed with the ligation product. Identity of the construct was confirmed by DNA sequencing of the Trichoderma reesei cellobiohydrolase I gene promoter, Humicola insolens endoglucanase V signal sequence, Humicola insolens endoglucanase V core sequence, Humicola insolens endoglucanase V signal sequence, Aspergillus oryzae beta-glucosidase mature coding sequence, and the Trichoderma reesei cellobiohydrolase I gene terminator sequence from plasmids purified from transformed E. coli. One clone containing the recombinant plasmid was designated pSaMe-FH. Plasmid pSaMe-FH comprises the Trichoderma reesei cellobiohydrolase I (CEL7A) gene promoter and terminator operably linked to the gene fusion of Humicola insolens CEL45A core/Aspergillus oryzae beta-glucosidase. Plasmid pSaMe-FH is identical to pSaMe-FX except the amdS selectable marker has been removed and replaced with the hygromycin resistance selectable marker.

### Example 28

### Isolation of Mutant of Trichoderma reesei SMA135-04 with Increased Cellulase Production and Enhanced Pretreated Corn Stover (PCS) Degrading Ability

PCS (Example 26) was used as a cellulose substrate for cellulolytic enzyme assays and for selection plates. Prior to assay, PCS was washed with a large volume of distilled deionized water until the filtrate pH was greater than pH 4.0. Also, PCS was sieved using 100MF metal filter to remove particles. The washed and filtered PCS was re-suspended in distilled water to a concentration of 60 mg/ml suspension, and stored at 4° C.

Trichoderma reesei strain SMA135-04 (Example 12) was subjected to mutagenic treatment with N-methyl-N-nitro-N-nitrosoguanidine (NTG) (Sigma Chemical Co., St. Louis, Mo., USA), a chemical mutagen that induces primarily base substitutions and some deletions (Rowlands, 1984, Enzyme Microb. Technol. 6: 3-10). Survival curves were done with a constant time of exposure and varying doses of NTG, and with a constant concentration of NTG and different times of exposure to get a survival level of 10%. To obtain this survival rate, a conidia suspension was treated with 0.2 mg/ml of NTG for 20 minutes at 37° C. with gentle rotation. Each experiment was conducted with a control where the conidia were not treated with NTG.

Primary selection of mutants was performed after the NTG treatment. A total of 8×10⁶ conidia that survived the mutagenesis were mixed in 30 ml of Mandel's medium containing 0.5% Peptone, 0.1% TRITON® X-100 and 1.5 g of agar. This suspension was then added to a deep plate (150 mm in diameter and 25 mm deep; Corning Inc., NY, USA) and the agar was allowed to harden at room temperature. After hardening the agar, 200 ml of Mandels medium containing 0.5% Peptone, 0.1% TRITON® X-100, 1.5% agar, and 1.0% PCS was added. The plates were incubated at 28° C. after hardening of the agar. After 3-5 days of incubation, 700 colonies that penetrated through the PCS selection layer before the non-treated control strain were used for secondary selection.

For secondary selection, three loopfuls of conidia from each isolate were added to 125 ml shake flasks containing 25 ml of cellulase-inducing medium and incubated at 28° C. and 200 rpm for 5 days to induce expression and secretion of cellulases. One ml of each culture broth was centrifuged at 400×g for 5 minutes in a microcentrifuge and the supernatants assayed for hydrolyzing activity of PCS and for total protein yield.

“Robotic” PCS hydrolysis assay was performed by diluting shake flask broth samples 1:20 in 50 mM sodium acetate pH 5.0. The diluted samples were added to assay plates (96 well flat-bottom plates) at 400 μl of sample per g of PCS before dilution. Using a BIOMEK® FX (Beckman Coulter, Fullerton, Calif., USA), PCS was added at 10 g of PCS per liter followed by 50 mM sodium acetate pH 5.0 to a total volume of 180 μl. The assay plates were incubated for 5 days at 30° C. in humidified boxes, which were shaken at 250 rpm. In order to increase the statistical precision of the assays, 6 replicates were performed for each sample. However, 2 replicates were performed for the 1:20 sample dilution. After 5 days incubation, the concentrations of reducing sugars (RS) in the hydrolyzed PCS samples were measured using a PHBAH assay, which was modified and adapted to a 96-well microplate format. Using an ORCA™ robot (Beckman Coulter, Fullerton, Calif., USA), the growth plates were transported to a BIOMEK® FX and 9 μl of broth samples were removed from the assay plates and aliquoted into 96-well V-bottom plates (MJ Research, Waltham, Mass., USA). The reactions were initiated by the addition of 135 μl of 0.533% PHBAH in 2% sodium hydroxide. Each assay plate was heated on a TETRAD® Thermal Cycler (MJ Research, Waltham, Mass., USA) for 10 minutes at 95° C., and cooled to room temperature. After the incubation, 40 μl of the reaction samples were diluted in 160 μl of deionized water and transferred into 96-well flat-bottom plates. Then, the samples were measured for absorbance at 405 nm using a SPECTRAMAX® 250 (Molecular Devices, Sunnyvale, Calif., USA). The A₄₀₅ values were translated into glucose equivalents using a standard curve generated with six glucose standards (0.000, 0.040, 0.800, 0.120, 0.165, and 0.200 mg per ml of deionized water), which were treated similarly to the samples. The average correlation coefficient for the standard curves was greater than 0.98. The degree of cellulose conversion to reducing sugar (RS yield, %) was calculated using the equation described in Example 26.

Total protein yield was determined using a bicinchoninic acid (BCA) assay. Samples were diluted 1:8 in water to bring the concentration within the appropriate range. Albumin standard (BSA) was diluted at various levels starting with a 2.0 mg/ml concentration and ending with a 0.25 mg/ml concentration in water. Using a BIOMEK® FX, a total of 20 μl of each dilution including standard was transferred to a 96-well flat bottom plate. Two hundred microliters of a BCA substrate solution (BCA Protein Assay Kit, Pierce, Rockford, Ill., USA) was added to each well and then incubated at 37° C. for 45 minutes. Upon completion of the incubation, the absorbance at 562 nm was measured for the 96-well plate using a SPECTRAMAX® 250. Sample concentrations were determined by extrapolation from the generated standard curve by Microsoft Excel (Microsoft Corporation, Redmond, Wash., USA).

Of the primary isolates picked, twenty produced broth that showed improved hydrolyzing activity of PCS when compared to broth from strain SMA135-04. These isolates produced cellulolytic broth that was capable of producing 5-15% higher levels of reducing sugar relative to the parental strain. Some isolates, for example, SMai-M104 showed increased performance in hydrolysis of cellulose PCS per volume broth, and additionally secreted higher levels of total protein.

Selection of the best performing Trichoderma reesei mutant strain, SMai-M104, was determined by assessing cellulase performance of broth produced by fermentation. The fermentation was run for 7 days as described in Example 26. The fermentation samples were tested in a 50 g PCS hydrolysis in 125-ml Erlenmeyer flasks with screw caps (VWR, West Chester, Pa., USA). Reaction conditions were cellulose loading of 6.7%; enzyme loadings of 6 and 12 mg/g cellulose; total reactants of 50 g; 50° C. and pH 5.0. Each shake flask and cap was weighed and the desired amount of PCS was added to the shake flask and the total weight was recorded. Ten ml of distilled water was added to each shake flask and then all the shake flasks were autoclaved for 30 minutes at 121° C. After autoclaving, the flasks were allowed to cool to room temperature. In order to adjust the total weight of each flask to 50 grams, 5 ml of 0.5 M sodium acetate pH 5.0 was added followed by broth to achieve the desired loading. Then the appropriate amount of distilled water was added to reach the desired final 50 g weight. The flasks were then placed in an incubator shaker (New Brunswick Scientific, Edison, N.J., USA) at 50° C. and 130 rpm. At days 3, 5 and 7, 1 ml samples were removed from each flask and added to a 96-deep-well plate (2.0 ml total volume). The 96 well-plate was then centrifuged at 3000 rpm for 15 minutes using a SORVALL® RT7 plate centrifuge (Thermo Fisher Scientific, Waltham, Mass., USA). Following centrifugation, 200 μl of supernatant was transferred to a 96-well 0.45 μm pore size filtration plate (Millipore, Bedford, Mass., USA) and vacuum applied in order to collect the filtrate. The filtrate was then diluted to a proper range of reducing sugars with 0.4% NaOH and measured using a PHBAH reagent (1.5%) as follows: 50 ul of the PHBAH reagent and 100 μl sample were added to a V-bottom 96-well plate and incubated at 95° C. for 10 minutes. To complete the reaction, 50 μl distilled water was added to each well and after mixing the samples, 100 μl of the mix was transferred to another flat-bottom 96-well plate to measure the absorbance at 410 nm. The reducing sugar amount was calculated using a glucose calibration curve and percent digestion was calculated as:

% digestion=reducing sugars(mg/ml)/(cellulose added(mg/ml)×1.11), where the factor 1.11 reflects the weight gain in converting cellulose to glucose.

The PCS hydrolysis assay results showed that one mutant, designated SMai-M104, slightly (approximately 5% increase in glucose) outperformed parental strain Trichoderma reesei SMA135-04, especially at high loading (12 mg/g cellulose).

### Example 29

### Construction of Trichoderma reesei strain SMai26-30

A co-transformation was utilized to introduce plasmids pCW085 (WO 2006/074435), pSaMe-FH, and pCW087 (Example 23) into Trichoderma reesei SMai-M104. Plasmid pCW085 is an expression vector for a Thielavia terrestris NRRL 8126 cellobiohydrolase (CEL6A). All three plasmids were introduced into Trichoderma reesei SMai-M104 by PEG-mediated transformation (Penttila et al., 1987, supra). Each plasmid contained the Escherichia coli hygromycin B phosphotransferase (hph) gene to enable transformants to grow on hygromycin B.

Trichoderma reesei SMai-M104 was cultivated at 27° C. and 90 rpm in 25 ml of YP medium supplemented with 2% (w/v) glucose and 10 mM uridine for 17 hours. Mycelia were collected by filtration using a Vacuum Driven Disposable Filtration System and washed twice with deionized water and twice with 1.2 M sorbitol. Protoplasts were generated by suspending the washed mycelia in 20 ml of 1.2 M sorbitol containing 15 mg of GLUCANEX® per ml and 0.36 units of chitinase per ml and incubating for 15-25 minutes at 34° C. with gentle shaking at 90 rpm. Protoplasts were collected by centrifuging for 7 minutes at 400×g and washed twice with cold 1.2 M sorbitol. The protoplasts were counted using a haemacytometer and re-suspended in STC to a final concentration of 1×10⁸ protoplasts per ml. Excess protoplasts were stored in a Cryo 1° C. Freezing Container at −80° C.

Approximately 10 μg each of plasmids pCW085, pSaMe-FH, and pCW087 were digested with Pme I and added to 100 μl of protoplast solution and mixed gently, followed by 260 μl of PEG buffer, mixed, and incubated at room temperature for 30 minutes. STC (3 ml) was then added and mixed and the transformation solution was plated onto PDA plates containing 1 M sucrose and 10 mM uridine. The plates were incubated at 28° C. for 16 hours, and then an agar overlay containing hygromycin B (30 μg/ml) final concentration) was added and incubation was continued for 4-6 days. Eighty transformants were subcultured onto PDA plates and grown at 28° C.

The Trichoderma reesei transformants were cultivated in 125 ml baffled shake flasks containing 25 ml of cellulase inducing medium at pH 6.0 inoculated with spores of the transformants and incubated at 28° C. and 200 rpm for 5 days. Trichoderma reesei SMai-M104 was run as a control. Culture broth samples were removed at day 5. One ml of each culture broth was centrifuged at 15,700×g for 5 minutes in a microcentrifuge and the supernatants transferred to new tubes.

SDS-PAGE was carried out using CRITERION® Tris-HCl (5% resolving) gels with a CRITERION® System. Five μl of day 5 supernatants (see above) were suspended in 2× concentration of Laemmli Sample Buffer and boiled in the presence of 5% beta-mercaptoethanol for 3 minutes. The supernatant samples were loaded onto a polyacrylamide gel and subjected to electrophoresis with 1× Tris/Glycine/SDS as running buffer. The resulting gel was stained with BIO-SAFE® Coomassie Blue Stain. Transformants showing expression of the Thermoascus aurantiacus GH61A polypeptide and the fusion protein consisting of the Humicola insolens endoglucanase V core (CEL45A) fused with the Aspergillus oryzae beta-glucosidase and Thielavia terrestris cellobiohydrolase II as seen by visualization of bands on SDS-PAGE gels were then tested in PCS hydrolysis reactions as described in Example 26 to identify the strains producing the best hydrolytic broths. One transformant that produced the highest performing broth was designated Trichoderma reesei SMai26-30.

Hydrolysis of PCS by Trichoderma reesei strain SMai26-30 broth was conducted as described in Example 26 with the following modifications. The lot of PCS was different than that used in Example 26, but prepared under similar conditions. In this protocol hydrolysis of PCS (approximately 11.3% in PCS and 6.7% cellulose in aqueous 50 mM sodium citrate pH 5.0 buffer) was performed using different protein loadings (expressed as mg of protein per gram of cellulose) of the Trichoderma reesei strain SMai26-30 fermentation broth. Aliquots were taken during the course of hydrolysis at 48, 120 and 168 hours. The results of the PCS hydrolysis reactions in the 50 g flask assay described above are shown in Table 3.

TABLE 3
Percent conversion to sugars at 48, 72 and 168 hours

| | Hours of hydrolysis | | |
|---|---|---|---|
| | 48 | 120 | 168 |
| mg/ml | Percent conversion | | |
| 2.52 | 47.2 | 60.4 | 64.1 |
| 2.52 | 48.2 | 61.1 | 64.8 |
| 5.01 | 67.2 | 85.0 | 87.7 |
| 5.01 | 67.9 | 85.8 | 88.8 |
| 9.98 | 85.2 | 95.4 | 96.0 |
| 9.98 | 85.3 | 93.6 | 94.7 |

Trichoderma reesei SMai26-30 was spore-streaked through two rounds of growth on plates to insure it was a clonal strain, and multiple vials frozen prior to production scaled in process-scale fermentor. Resulting protein broth was recovered from fungal cell mass, filtered, concentrated and formulated. The cellulolytic enzyme preparation was designated Cellulolytic Enzyme Composition #2.

### Example 30

### Effect of a Mixture of Tannic Acid, Ellagic Acid, Epicatechin, and Various Lignin Constituent Compounds on PCS Hydrolysis

Corn stover was pretreated at the U.S. Department of Energy National Renewable Energy Laboratory (NREL), Boulder, Colo., USA, using dilute sulfuric acid. The following conditions were used for the pretreatment: 1.4 wt % sulfuric acid at 195° C. for 4.5 minutes. According to limit digestion with excess cellulase enzymes, the water-insoluble solids in the pretreated corn stover (PCS) contained 59.5% cellulose. Prior to use, the PCS was washed with a large volume of deionized water until soluble acid and sugars were removed. The dry weight of the water-washed PCS was 19.16%.

The effect of a mixture of tannic acid, ellagic acid, epicatechin, and six lignin constituent compounds (4-hydroxyl-2-methylbenzoic acid, vanillin, coniferyl alcohol, coniferyl aldehyde, ferulic acid, and syringaldehyde) was determined on the hydrolysis of PCS by Cellulolytic Enzyme Composition #1 or Cellulolytic Enzyme Composition #2. The PCS hydrolysis reactions were performed in duplicate in capped 1.7 ml EPPENDORF® tubes (“mini-scale”) containing 1 ml suspensions of 43.4 g of PCS (dry weight) per liter of 50 mM sodium acetate pH 5.0, 1 mM tannic acid (corresponding to 10 mM galloyl and 1 mM glucosyl constituents), 1 mM ellagic acid, 1 mM epicatechin, and a lignin constituent mixture of 1 mM 4-hydroxyl-2-methylbenzoic acid, 1 mM vanillin, 1 mM coniferyl alcohol, 1 mM coniferyl aldehyde, 1 mM ferulic acid, and 1 mM syringaldehyde in the same buffer. Cellulolytic Enzyme Composition #1 or Cellulolytic Enzyme Composition #2 was added at 0.25 g per liter. Reactions without the addition of the compounds served as controls. The capped tubes were incubated at 50° C. in an INNOVA® 4080 incubator shaker (New Brunswick Scientific Co., Inc., Edison, N.J., USA) at 150 rpm.

Aliquots of the suspensions, sampled over time, were filtered by centrifugation using a 0.45 μm MULTISCREEN® HV membrane (Millipore, Billerica, Mass., USA) at 2000 rpm for 15 minutes using a SORVALL® RT7 centrifuge (Thermo Fisher Scientific, Waltham, Mass., USA). When not used immediately, the filtered aliquots were frozen at −20° C. Sugar concentrations of the samples diluted in 0.005 M H₂SO₄ were measured after elution by 0.005 M H₂SO₄ at a flow rate of 0.4 ml/minute from a 4.6×250 mm AMINEX® HPX-87H column (Bio-Rad, Hercules, Calif., USA) at 65° C. with quantitation by integration of glucose and cellobiose using refractive index detection (CHEMSTATION®, AGILENT® 1100 HPLC, Agilent Technologies, Santa Clara, Calif., USA) calibrated with standards of glucose and cellobiose. The resultant equivalents were used to calculate the percentage of cellulose conversion for each reaction.

The degree of cellulose conversion to glucose plus cellobiose sugars (conversion, %) was calculated using the following equation:

Conversion(%)=(glucose+cellobiose×1.053)(mg/ml)×100×162/cellulose(mg/ml)×180)=(glucose+cellobiose×1.053)(mg/ml)×100/(cellulose(mg/ml)×1.111)

In this equation the factor 1.111 reflects the weight gain in converting cellulose to glucose, and the factor 1.053 reflects the weight gain in converting cellobiose to glucose. Cellulose in PCS was determined by a limit digest of PCS to release glucose and cellobiose.

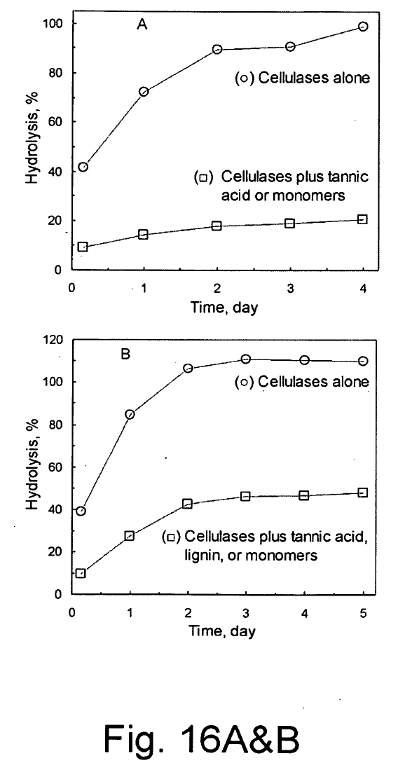

The results shown in FIGS. 16A and 16B demonstrated that the mixture significantly inhibited the hydrolysis of PCS by either Cellulolytic Enzyme Composition #1 or Cellulolytic Enzyme Composition #2.

### Example 31

### Effect of Tannic Acid, Ellagic Acid, Epicatechin, and Various Lignin Constituent Compounds on PCS Hydrolysis

Example 30 was repeated except that each compound was tested separately. Soluble reducing sugars were measured by HPLC as described in Example 30. Reactions without the addition of each compound served as controls.

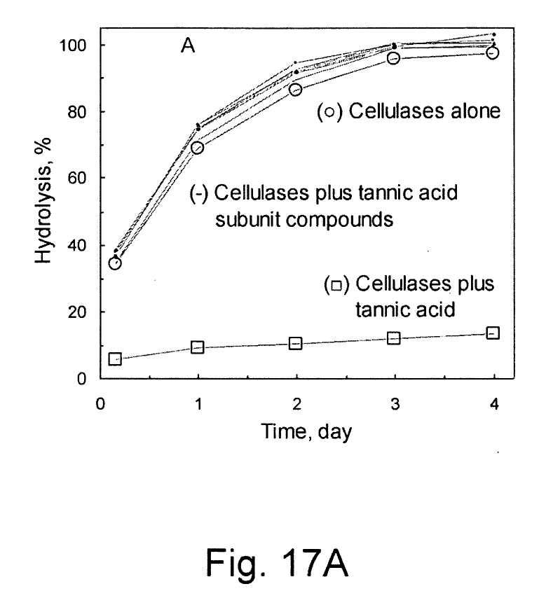

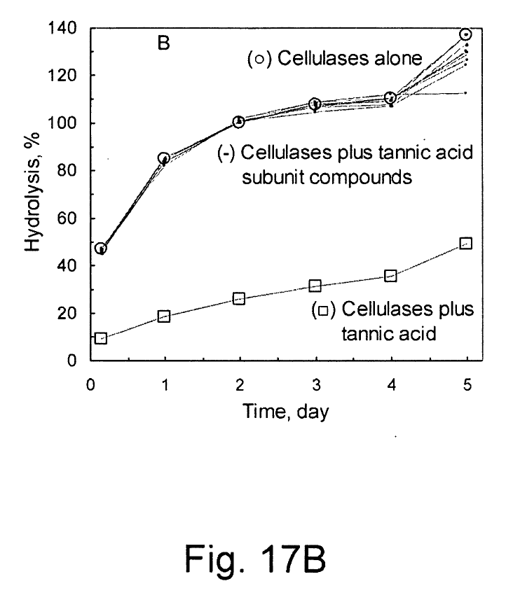

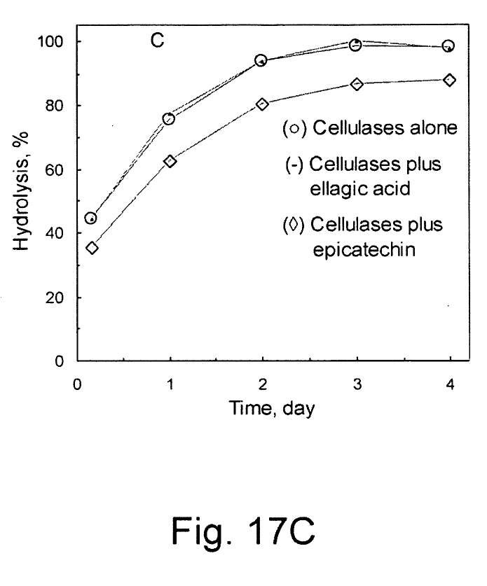

The results shown in FIGS. 17A, 17B, and 17C demonstrated that only tannic acid (FIG. 17A), but not its constituent ellagic acid (FIG. 17C), significantly inhibited the hydrolysis of PCS, while all of the lignin/tannin constituent compounds at 1 mM were not inhibitory. There was a slight inhibition of Cellulolytic Enzyme Composition #1 by 1 mM epicatechin (FIG. 17C).

### Example 32

### Effect of Condensed Tannin (OPC) and Constituent Compounds on PCS Hydrolysis

The effect of OPC or flavonol on the hydrolysis of PCS by Cellulolytic Enzyme Composition #1 or Cellulolytic Enzyme Composition #2 was determined according to the procedure described in Example 30. OPC and flavonol were present at a concentration of 1 mM. Reactions without the addition of the compounds served as controls. Soluble reducing sugars were measured by HPLC as described in Example 30. Since OPC contained hydrolyzable glycans from the inactive ingredients used in the OPC tablets, the effect of the OPC was estimated after subtracting the sugars derived when PCS was absent from the hydrolysis.

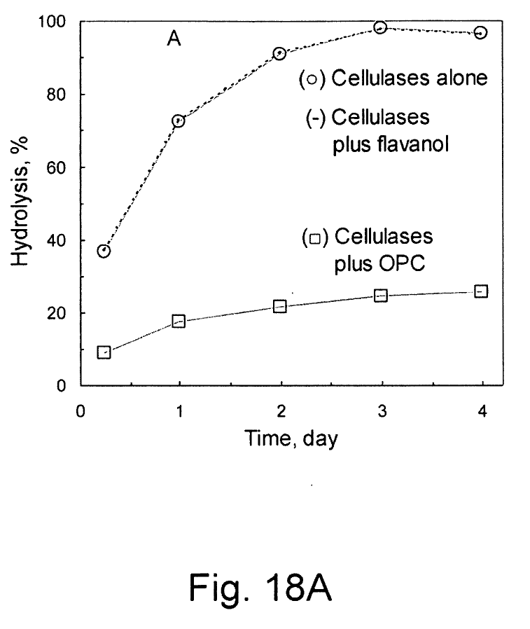

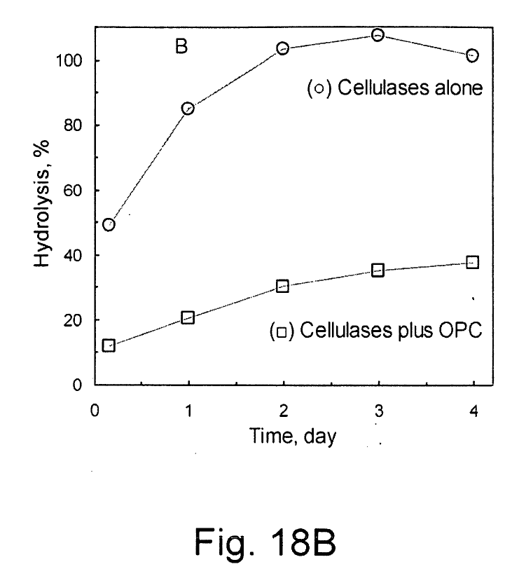

The results shown in FIGS. 18A and 18B demonstrated that only OPC, and not its constituent flavonol, was inhibitory to Cellulolytic Enzyme Composition #1. Flavonol was also not inhibitory to Cellulolytic Enzyme Composition #2.

### Example 33

### Concentration Dependence of Tannic Acid and OPC Inhibition

The effective inhibitory concentration range of tannic acid and OPC was determined by hydrolysis of AVICEL® by Cellulolytic Enzyme Composition #1.

The hydrolysis involving tannic acid was performed in duplicate using the “mini-scale” hydrolysis reaction procedure described in Example 30, except that 0.05 mM to 1 mM tannic acid and 23 g of AVICEL® (dry weight) per liter of 50 mM sodium acetate pH 5.0 was used. The hydrolysis involving OPC was performed in duplicate in a 2.8 ml 96-well Deep Well Microplates (VWR International, West Chester, Pa.) (“mini-plate-scale”) containing 1 ml suspensions of 1 mM to 10 mM OPC and 23 g of AVICEL® (dry weight) per liter of 50 mM sodium acetate pH 5.0. Cellulolytic Enzyme Composition #1 was added at 0.25 g per liter for each hydrolysis. The mini-plates were sealed at 160° C. for 2 seconds using an ALPS 300™ sealer. Reactions without the addition of the aromatic compounds served as controls. The capped tubes or sealed mini-plates were incubated at 50° C. in a New Brunswick Scientific Innova 4080 incubation shaker at 150 rpm. Soluble reducing sugars were measured by HPLC as described in Example 30.

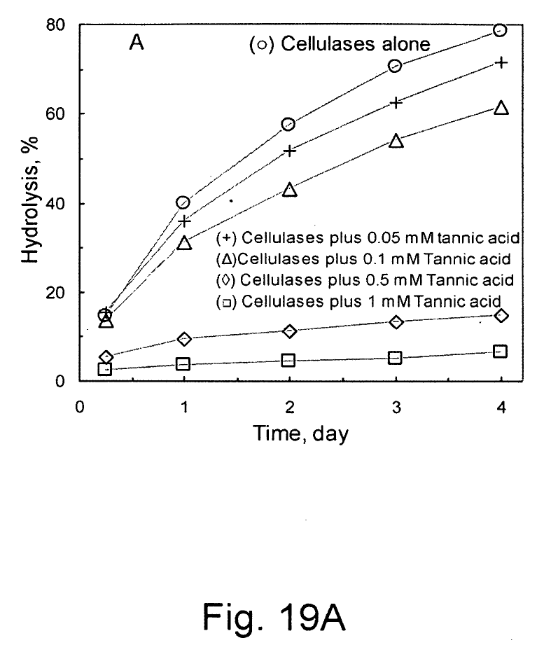

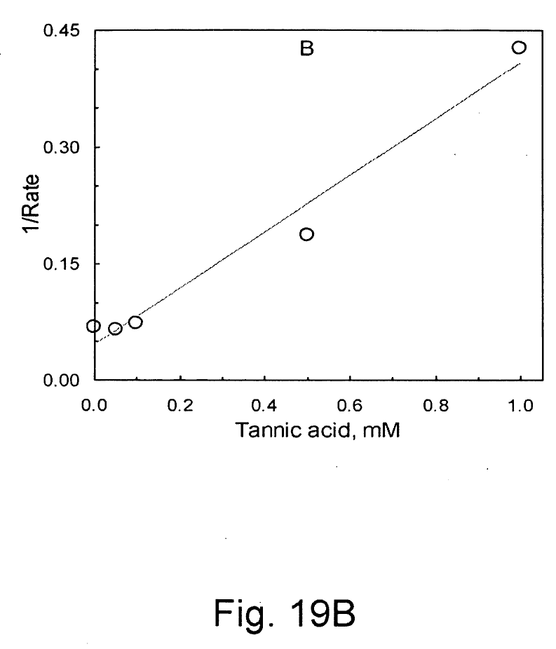

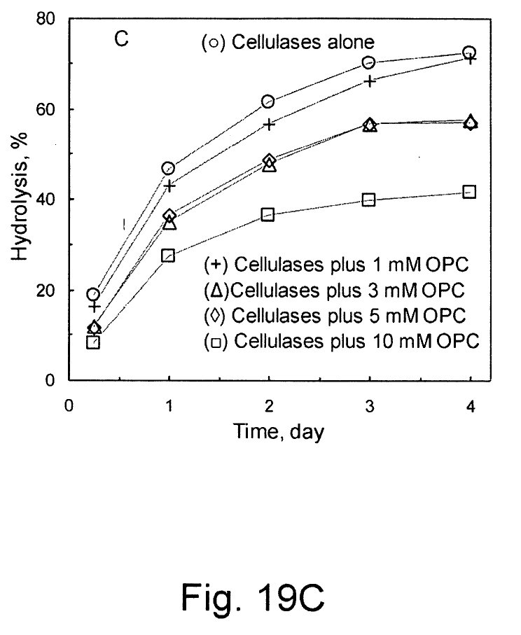

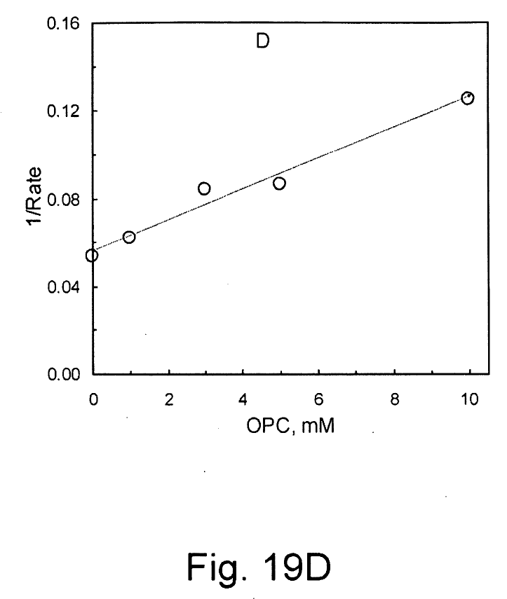

The results as shown in FIGS. 19A and 19C demonstrated that tannic acid was increasingly inhibitory over the concentration range of 0.05 mM to 1 mM tannic acid (FIG. 19A), while OPC was increasingly inhibitory over the concentration range of 1 mM to 10 mM (FIG. 19C). Dixon plots (inverse of initial rate vs inhibitor concentration) indicated an inhibition constant Kᵢ (x-intercept) of approximately 0.13 mM for tannic acid (FIG. 19B) and approximately 8 mM for OPC (FIG. 19D).

The effective inhibitory concentration range for tannic acid and OPC was also determined by the “mini-scale” hydrolysis described in Example 30 with Cellulolytic Enzyme Composition #2. The concentration of tannic acid ranged from 0.1 mM to 1 mM, while the concentration of OPC ranged from 0.1 mM to 10 mM. Reactions without the addition of the tannic compounds served as controls. Soluble reducing sugars were measured by HPLC as described in Example 30.

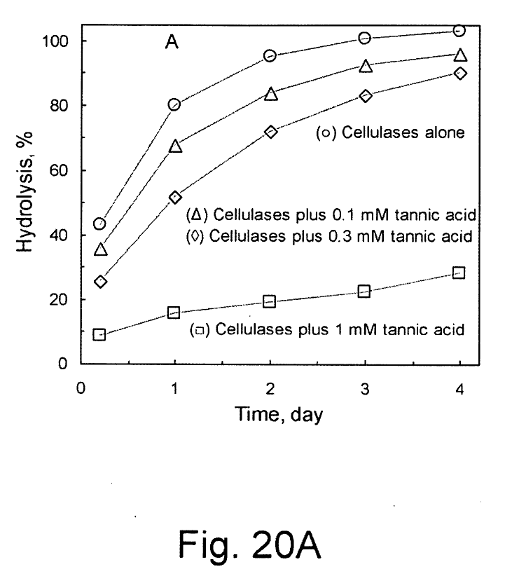

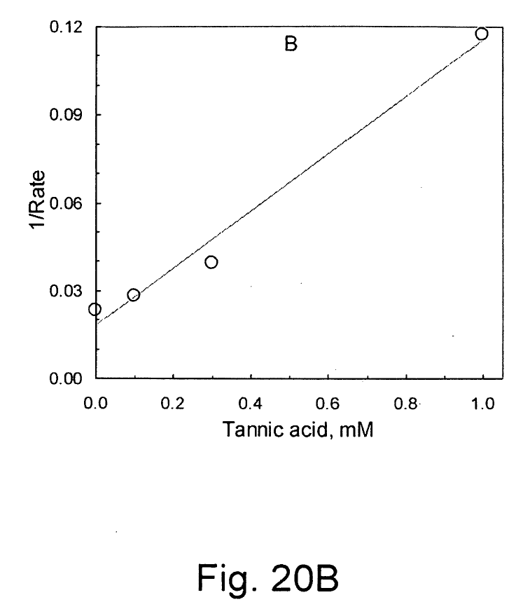

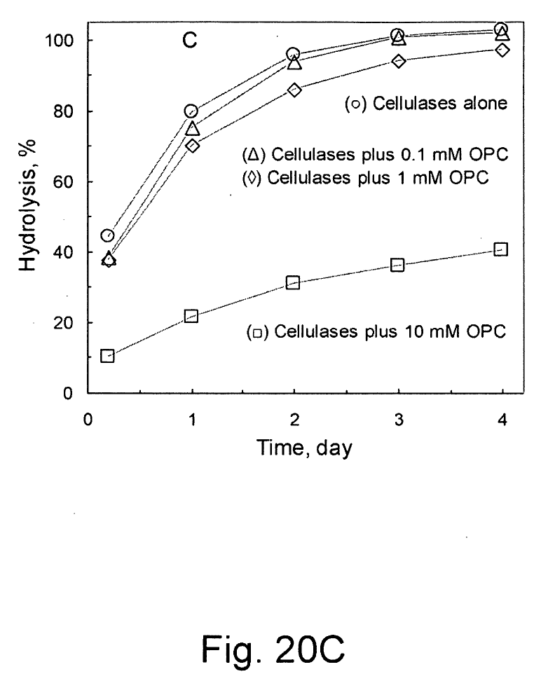

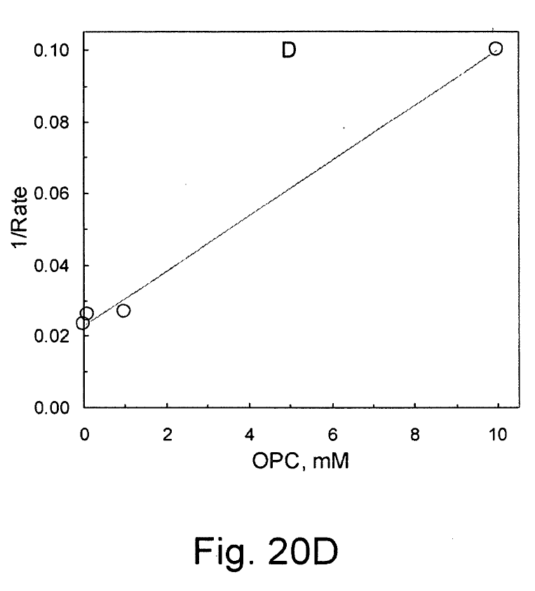

The results as shown in FIGS. 20A and 20C demonstrated that tannic acid was increasingly inhibitory over the concentration range of 0.1 mM to 1 mM (FIG. 20A), while OPC was increasingly inhibitory over the concentration range of 0.1 mM to 10 mM (FIG. 20C). Dixon plots indicated a Kᵢ (x-intercept) of approximately 0.18 mM for tannic acid (corresponding to 1.8 mM galloyl constituents) (FIG. 20B) and approximately 2.9 mM for OPC (flavonol-equivalent) (FIG. 20D).

### Example 34

### Inhibitory Effect of Tannic Acid's Constituents on Hydrolysis of AVICEL®

To further examine how tannic acid inhibits enzymatic hydrolysis of cellulose, hydrolysis of AVICEL® by Cellulolytic Enzyme Composition #1 was evaluated with or without 10 mM methyl gallate plus 1 mM glucose pentaacetate, or 5 mM ellagic acid plus 1 mM glucose pentaacetate, both combinations mimicking 1 mM tannic acid. The hydrolysis reactions were conducted according to the “mini-plate-scale” hydrolysis procedure described Example 33 with 25 g of AVICEL® and 0.25 g of Cellulolytic Enzyme Composition #1 per liter of 50 mM sodium acetate pH 5.0 at 50° C. Soluble sugars were measured by HPLC as described in Example 30.

The results demonstrated that the ellagic acid plus glucose pentaacetate mix yielded approximately a 20% loss in initial rate but no loss in the extent of hydrolysis at day 8, while the methyl gallate plus glucose pentaacetate mix yielded approximately a 20% loss in both initial rate and the extent of hydrolysis at day 8. In contrast, tannic acid yielded approximately a 90% loss in initial rate and a 70% loss in the extent of hydrolysis at day 8, suggesting the importance of the structure of tannic acid, rather than composition, in inhibition.

### Example 35

### Effect of Tannic Acid's Constituents on Enzymatic PCS Hydrolysis

Methyl gallate and ellagic acid were compared at 10 mM to 1 mM tannic acid in the hydrolysis of PCS by Cellulolytic Enzyme Composition #1. The hydrolysis reactions were conducted according to the “mini-plate-scale” procedure described Example 33 with 50 g of PCS and 0.25 g of Cellulolytic Enzyme Composition #1 per liter of 50 mM sodium acetate pH 5.0 at 50° C. Soluble reducing sugars were measured by HPLC as described in Example 30.

The results demonstrated that ellagic acid yielded approximately a 30% loss in initial rate and 40% loss in the extent of hydrolysis at day 4, while methyl gallate yielded approximately a 10% loss in both initial rate and the extent of hydrolysis at day 4. In contrast, the tannic acid yielded approximately a 70% loss in initial rate and 60% loss in the extent of hydrolysis at day 4.

### Example 36

### Inhibition Constants of Tannic Acid

Tannic acid's inhibition of Cellulolytic Enzyme Composition #1 was quantified by a series of hydrolysis reactions performed according to the “mini-plate-scale” hydrolysis procedure described in Example 33 with 0.6 to 4 g of PASC or AVICEL® and 0.01 g of Cellulolytic Enzyme Composition #1 per liter of 50 mM sodium acetate pH 5.0, and 0.1 to 0.7 mM tannic acid at 50° C. Soluble sugars were measured by HPLC as described in Example 30. Initial hydrolysis rates were obtained from the first two hydrolysis time points (i.e., soluble sugar measurements) (with <20% hydrolysis extent in general, rate=(hydrolysis difference)/(time difference)). Double-reciprocal plots (1/(initial rate) vs 1/[cellulose] as function of tannic acid concentration) indicated a “mixed” type inhibition, but their complexity prevented extraction of simple inhibitor constants. Initial rate vs tannic acid concentration yielded an I₅₀ (inhibitor concentration leading to 50% loss of hydrolysis rate) of 0.2±0.1 or 0.27±0.07 mM on PASC or AVICEL® hydrolysis, respectively

### Example 37

### Inhibitory Effect of Tannic Acid on Individual Cellulolytic Enzymes

The inhibitory effect of tannic acid was determined on Trichoderma reesei CEL7A cellobiohydrolase 1, Trichoderma reesei CEL6A cellobiohydrolase II, Trichoderma reesei CEL7B endoglucanase 1, and Trichoderma reesei CEL5A endoglucanase II using PASC as substrate.

The hydrolysis was performed in a series of duplicate “mini-plate-scale” hydrolysis reactions according to the procedure described in Example 33, except that 1 mM tannic acid (corresponding to 10 mM galloyl and 1 mM glucosyl constituents) and 2 g of PASC (dry weight) and 0.5 g of bovine serum albumin (BSA) per liter of 50 mM sodium acetate pH 5.0 was used.

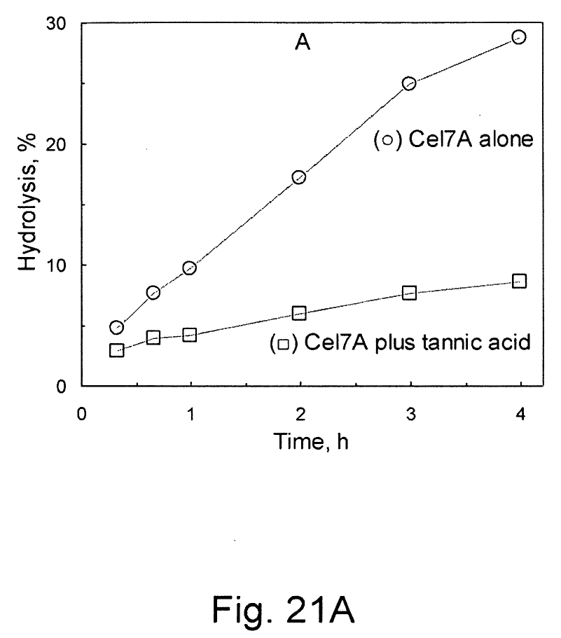

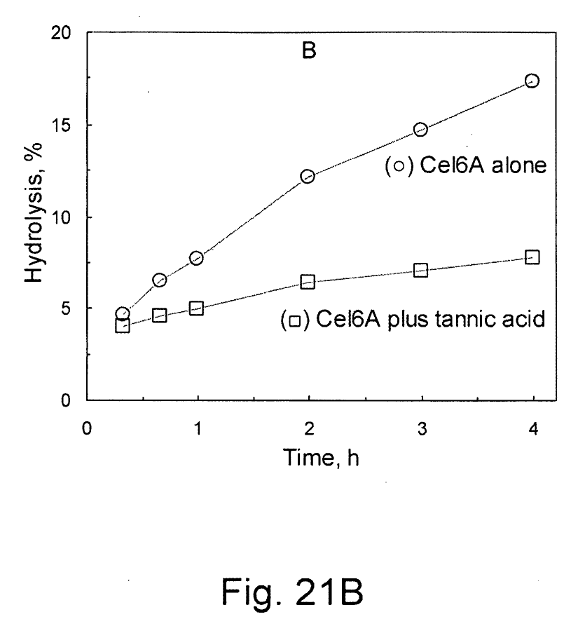

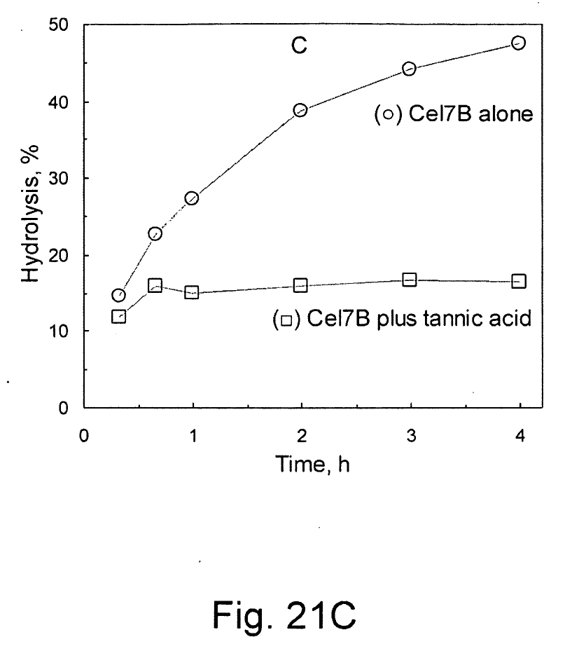

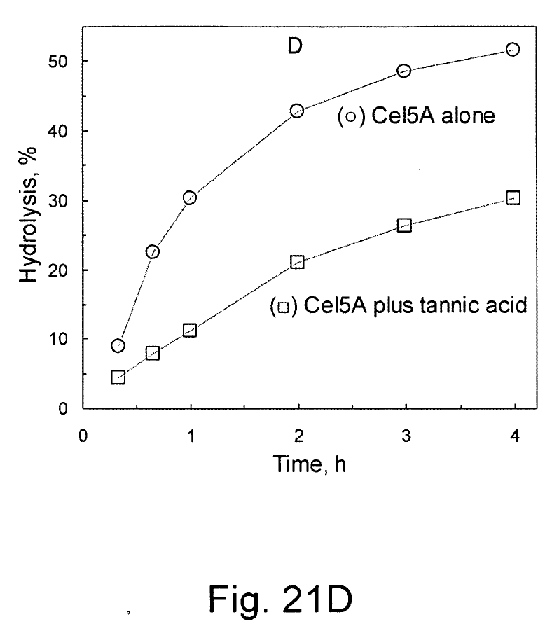

The results as shown in FIGS. 21A, 21B, 21C, and 21D demonstrated that tannic acid significantly inhibited the Trichoderma reesei enzymes. No hydrolysis of PASC was observed with tannic acid alone.

The effect of tannic acid on Trichoderma reesei CEL7B endoglucanase I and Trichoderma reesei CEL5A endoglucanase II was also evaluated using carboxymethylcellulose (CMC) as substrate. The hydrolysis reactions were conducted in duplicate using the “mini-plate-scale” hydrolysis procedure described in Example 33, except that 1 mM tannic acid and 10 to 20 g of carboxymethylcellulose (CMC) and 1 to 20 mg of enzyme per liter 50 mM sodium acetate pH 5.0 were used at 50° C. for 4 hours. Soluble reducing sugars were analyzed by a p-hydroxybenzoic acid hydrazide (PHBAH) assay according to the method of Lever, 1972, Anal. Biochem. 47: 273-279, instead of by HPLC as described in Examples 30 and 33. Reactions without the addition of the enzymes served as controls to correct background absorption. Spectrophotometric measurements were performed using a SPECTRAMAX™ 340PC reader (Molecular Devices Corp., Sunnyvale, Calif., USA) with COSTAR® 96-well microplates (Cole-Parmer Instrument Co, Vernon Hills, Ill., USA).

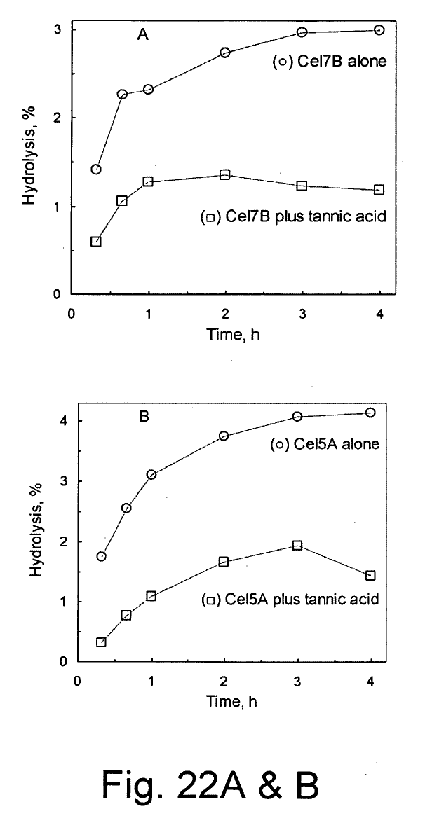

The results as shown in FIGS. 22A and 22B demonstrated that tannic acid significantly inhibited both enzymes, consistent with the results observed for the hydrolysis of PASC described above.

The effect of tannic acid on Aspergillus oryzae CEL3A beta-glucosidase was also evaluated using a series of “mini-scale” hydrolysis reactions according to the procedure described in Example 30, except that 1 mM tannic acid (corresponding to 10 mM galloyl and 1 mM glucosyl constituents) and 2 g of cellobiose and 1 mg of beta-glucosidase per liter of 39 mM sodium acetate pH 5.0 were used. Reactions without the addition of the tannic acid served as controls. The reaction was monitored by HPLC as described in Example 30.

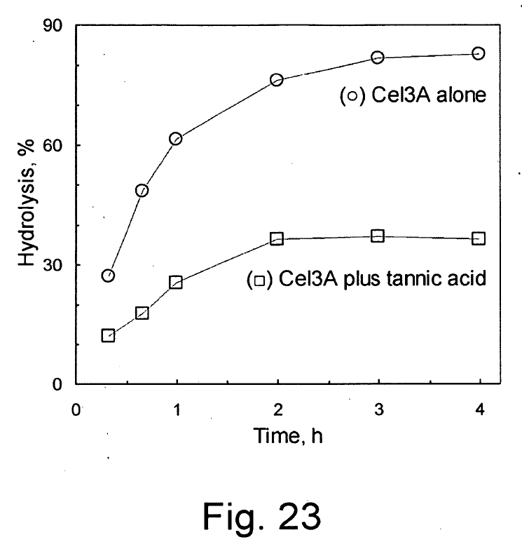

The results as shown in FIG. 23 demonstrated that tannic acid significantly inhibited Aspergillus oryzae CEL3A beta-glucosidase.

### Example 38

### Inhibition of Tannic Acid on Individual Cellulase-Catalyzed Cellulolysis

Example 37 showed that tannic acid inhibits the hydrolytic activity of various cellulase enzymes. To quantify the inhibition, tannic acid was evaluated in the hydrolysis of PASC. The hydrolysis reactions were conducted according to the “mini-plate-scale” hydrolysis procedure described in Example 33 with 0.1 to 0.7 mM tannic acid, and 0.6 to 4 g of PASC and 0.04 g of Trichoderma reesei CEL7A CBHI, CEL7B EGI, or CEL5A EGII per liter of 50 mM sodium acetate pH 5 at 50° C. Soluble sugars were measured by HPLC as described in Example 30.

Double reciprocal plots (as described in Example 36) indicated a “mixed” type inhibition, but their complexity prevented extraction of simple inhibitor constants. As shown in Table 4, initial rate versus tannic acid concentration suggested an 150 of approximately 1, 0.3±0.2, or 0.32±0.05 mM for CEL7A CBHI, CEL7B EGI, or CEL5A EGII, respectively.

Tannic acid was also evaluated in the hydrolysis of cellobiose. The hydrolysis reactions were conducted according to the “mini-plate-scale” hydrolysis procedure described in Example 33 with 0.6 to 4 g of cellobiose and 0.001 g of Aspergillus oryzae CEL3A beta-glucosidase per liter of 50 mM sodium acetate pH 5 at 50° C. The results indicated that the inhibition appeared to be mixed, with an I₅₀ of approximately 0.8 mM (Table 4).

TABLE 4
Inhibition parameter I₅₀ (mean ± SD, in mM) of tannic acid on enzymatic cellulolysis

| | Cellulolytic Enzyme | | CEL6A | CEL7B | CEL5A | |
|---|---|---|---|---|---|---|
| | Composition #1 | CEL7A CBH-I | CBH-II | EG-I | EG-II | CEL3A BG |
| PASC | 0.2 ± 0.1 | approximately 1 | ND | 0.3 ± 0.2 | 0.32 ± 0.05 | approximately |
| | | | | | | 0.8 |

ND: Not determined.

### Example 39

### Target of Tannic Acid or OPC Inhibition of Cellulose Hydrolysis

To examine where tannic acid exerted its inhibition, a series of hydrolysis reactions of AVICEL® by Cellulolytic Enzyme Composition #1 was performed in which AVICEL® and Cellulolytic Enzyme Composition #1 were used fresh or after pre-incubation with tannic acid. The hydrolysis reactions were conducted according to the “mini-plate-scale” hydrolysis procedure described in Example 33 with 25 g of AVICEL® and 0.25 g of Cellulolytic Enzyme Composition #1 per liter of 50 mM sodium acetate pH 5.0 at 50° C. After pre-incubation of 0.25 g of Cellulolytic Enzyme Composition #1 per liter of 50 mM sodium acetate pH 5.0 with 1 mM tannic acid for 1 hour at 50° C. (with detectable precipitation seen), the pre-incubated Cellulolytic Enzyme Composition #1 was gel-filtered using BioSpin 6 desalting columns (Bio-Rad, Hercules, Calif., USA). After pre-incubation of 25 g of AVICEL® per liter of 50 mM sodium acetate pH 5.0 with 1 mM tannic acid for 1 hour at 50° C., the pre-incubated AVICEL® with tannic acid was extensively washed with 50 mM sodium acetate pH 5 buffer. Hydrolysis of untreated or buffer-only pre-incubated AVICEL® and Cellulolytic Enzyme Composition #1, with or without inhibitors, served as controls.

Adding 1 mM tannic acid to fresh Cellulolytic Enzyme Composition #1 and AVICEL® mixture caused approximately a 90% loss in initial rate and a 70% loss in the extent of hydrolysis after 8 days. Pre-incubating AVICEL® with tannic acid did not affect the hydrolysis. In contrast, pre-incubating Cellulolytic Enzyme Composition #1 showed significantly reduced activity (approximately 80% loss). Since detectable precipitation occurred during the pre-incubation, suggesting complexation of the cellulase enzyme components with tannic acid, the activity loss was likely attributable to complexing and consequent protein loss during gel-filtration.

OPC was also evaluated as described above. After pre-incubation of 0.25 g of Cellulolytic Enzyme Composition #1 or 25 g of AVICEL® per liter of 50 mM sodium acetate pH 5.0 with 10 mM OPC (in subunits) for 1 hour at 50° C., followed by gel-filtration or washing, pre-incubated Cellulolytic Enzyme Composition #1 and AVICEL® with tannic acid showed no significant difference (<10%) from buffer-pre-incubated Cellulolytic Enzyme Composition #1 and AVICEL® in terms of hydrolysis (“mini-plate-scale” procedure described in Example 33), indicating no or a reversible (if any) modification on AVICEL® or Cellulolytic Enzyme Composition #1 by OPC.

### Example 40

### Reduction of Tannin or OPC Inhibition by Tannase

Tannase was evaluated for its ability to reduce the inhibitory effect of tannic acid on OPC on PCS hydrolysis by Cellulolytic Enzyme Composition #2.

The hydrolysis was performed in duplicate using the “mini-plate-scale” hydrolysis procedure described in Example 33 except that 1 mM tannic acid or 10 mM OPC and 43 g of PCS per liter, 25 mg of Cellulolytic Enzyme Composition #2 per liter of 50 mM sodium acetate pH 5.0 at 50° C. for 4 hours was used. However, prior to the addition of Cellulolytic Enzyme Composition #2, the mixture of PCS or OPC and tannic acid was treated with Aspergillus oryzae tannase (Novozymes A/S, Bagsværd, Denmark) at 10% of the final protein level for 30 minutes. Reactions without addition of the tannic acid, OPC, or tannase served as controls. Soluble reducing sugars were measured by HPLC as described in Example 30.

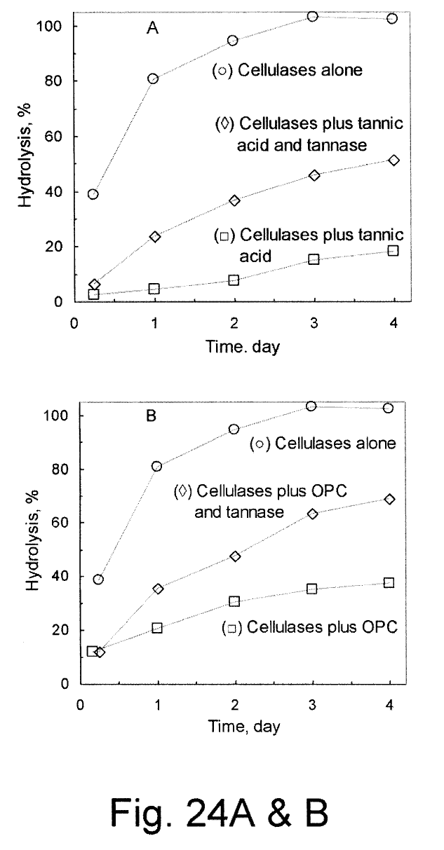

The results, as shown in FIGS. 24A and 24B, demonstrated that pretreatment of tannic acid and OPC with the Aspergillus oryzae tannase significantly reduced the inhibitory effect of tannic acid and OPC on Cellulolytic Enzyme Composition #2. In the absence of tannic acid or OPC, tannase alone slightly enhanced (approximately 2% increase in hydrolysis extent) PCS hydrolysis by Cellulolytic Enzyme Composition #2.

### Example 41

### Reduction of Tannic Acid Inhibition by Tannase

Example 40 showed that tannase mitigates tannic acid inhibition of cellulose hydrolysis by Cellulolytic Enzyme Composition #2. The effective concentration range for tannase was studied using the “mini-plate-scale” hydrolysis procedure described in Example 33, except that 43.4 g of PCS and 0.25 g of Cellulolytic Enzyme Composition #1 per liter of 50 mM sodium acetate pH 5.0 at 50° C. in the presence and absence of 1 mM tannic acid for up to 4 days. To reduce the inhibition, tannase was added at 12.5, 25, and 50 mg per liter (or 0.21, 0.42, and 0.85 μM).

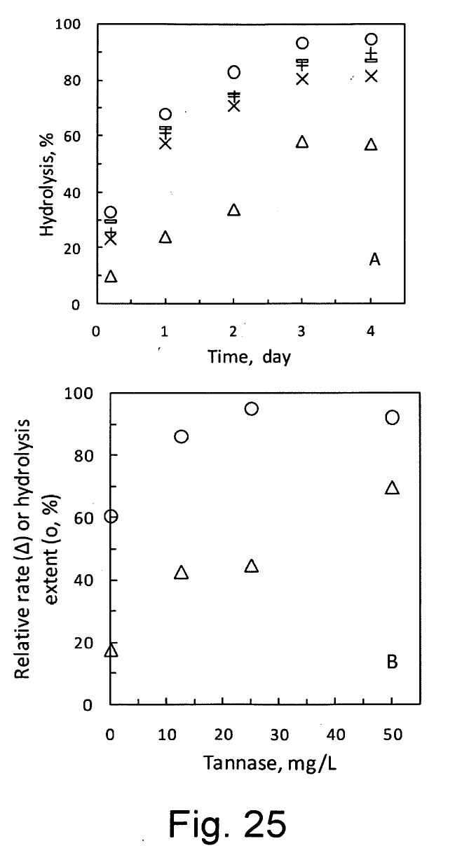

The results, as shown by FIG. 25, demonstrated that tannase reduced tannic acid inhibition in a dose-dependent manner, reaching approximately 50 or 100% reduction at approximately 12 or 25 mg per liter, respectively.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

Various references are cited herein, the disclosures of which are incorporated by reference in their entireties.

1. A method of producing a cellulosic material reduced in a tannin, comprising treating the cellulosic material with an effective amount of a tannase to reduce the inhibitory effect of the tannin on enzymatically saccharifying the cellulosic material.

2. (canceled)

3. The method of claim 1, wherein the treating of the cellulosic material with the tannase is performed at a pH in the range of about 2 to about 11.

4. (canceled)

5. (canceled)

6. The method of claim 1, wherein the treating of the cellulosic material with the tannase is performed at a temperature in the range of about 20° C. to about 90° C.

7. (canceled)

8. (canceled)

9. The method of claim 1, wherein the effective amount of the tannase is in the range of about 0.1 to about 10,000 units per g of dry cellulosic material.

10. (canceled)

11. (canceled)

12. The method of claim 1, wherein the cellulosic material is treated with the tannase before, during, and/or after the pretreatment and/or during saccharification and/or during a fermentation.

13. A method of saccharifying a cellulosic material, comprising: treating the cellulosic material with an effective amount of a tannase and an effective amount of a cellulolytic enzyme composition, wherein the treating of the cellulosic material with the tannase reduces the inhibitory effect of a tannin on enzymatically saccharifying the cellulosic material with the cellulolytic enzyme composition.

14. The method of claim 13, wherein the cellulosic material is pretreated before saccharification.

15. The method of claim 13, wherein the cellulosic material is treated with the tannase before, during, and/or after a pretreatment and/or during the saccharification.

16. (canceled)

17. The method of claim 13, wherein the treating of the cellulosic material with the tannase is performed at a pH in the range of about 2 to about 11.

18. (canceled)

19. (canceled)

20. The method of claim 13, wherein the treating of the cellulosic material with the tannase is performed at a temperature in the range of about 20° C. to about 90° C.

21. (canceled)

22. (canceled)

23. The method of claim 13, wherein the effective amount of the tannase is in the range of about 0.1 to about 10,000 units per g of dry cellulosic material.

24. (canceled)

25. (canceled)

26. The method of claim 13, wherein the cellulolytic enzyme composition comprises polypeptides having endoglucanase, cellobiohydrolase, and beta-glucosidase activities.

27. (canceled)

28. (canceled)

29. The method of claim 13, further comprising recovering the degraded cellulosic material.

30. (canceled)

31. (canceled)

32. A method of producing a fermentation product, comprising: (a) saccharifying a cellulosic material with an effective amount of a cellulolytic enzyme composition; (b) fermenting the saccharified cellulosic material of step (a) with one or more fermenting microorganisms to produce a fermentation product; and (c) recovering the fermentation product, wherein the cellulosic material is treated with an effective amount of a tannase to reduce the inhibitory effect of a tannin on enzymatically saccharifying the cellulosic material.

33. The method of claim 32, wherein the cellulosic material is pretreated before the saccharifying step.

34. The method of claim 32, wherein the cellulosic material is treated with the tannase before, during, and/or after a pretreatment and/or during the saccharification and/or during the fermentation.

35. (canceled)

36. The method of claim 32, wherein the treating of the cellulosic material with the tannase is performed at a pH in the range of about 2 to about 11.

37. (canceled)

38. (canceled)

39. The method of claim 32, wherein the treating of the cellulosic material with the tannase is performed at a temperature in the range of about 20° C. to about 90° C.

40. (canceled)

41. (canceled)

42. The method of claim 32, wherein the effective amount of the tannase is in the range of about 0.1 to about 10,000 units per g of dry cellulosic material.

43. (canceled)

44. (canceled)

45. The method of claim 32, wherein the cellulolytic enzyme composition comprises polypeptides having endoglucanase, cellobiohydrolase, and beta-glucosidase activities.

46. (canceled)

47. (canceled)

48. (canceled)

49. (canceled)
